(19) United States
(12) Patent Application Publication
(10) Pub. No.: US 2010/0286186 A1
(43) Pub. Date: Nov. 11, 2010

(54) NOVEL DICARBOXYLIC ACID LINKED AMINO ACID AND PEPTIDE PRODRUGS OF OPIOIDS AND USES THEREOF

(75) Inventors: Richard Franklin, Fleet (GB); Bernard T. Golding, Newcastle upon Tyne (GB); Robert G. Tyson, Durham (GB)

Correspondence Address: MCDERMOTT WILL & EMERY LLP, 600 13TH STREET, N.W., WASHINGTON, DC, 20005-3096, US

(73) Assignee: Shire LLC, Florence, KY (US)

(21) Appl. No.: 12/753,042

(22) Filed: Apr. 1, 2010

Related U.S. Application Data
(60) Provisional application No. 61/211,831, filed on Apr. 2, 2009.
(60) Provisional application No. 61/227,716, filed on Jul. 22, 2009.

(51) Int. Cl. A61K 31/485; C07D 489/08; C07D 489/04; A61P 1/00; A61P 25/36; A61P 43/00

(52) U.S. Cl. 514/282; 546/44

(57) ABSTRACT

The present invention concerns dicarboxylic acid linked amino acid and peptide prodrugs of opioid analgesics and pharmaceutical compositions containing such prodrugs. Methods for providing pain relief, decreasing the adverse GI side effects of the opioid analgesic and increasing the bioavailability of the opioid analgesic with the aforementioned prodrugs are also provided. In one embodiment, prodrugs having the amino acid side chains of valine, leucine, isoleucine and glycine; and mono-, di- and tripeptides thereof are provided.

### CROSS-REFERENCE TO RELATED APPLICATIONS

This application claims benefit under 35 U.S.C. §119(e) to U.S. Provisional Application No. 61/211,831 filed on Apr. 2, 2009 and claims benefit under 35 U.S.C. §119(e) to U.S. Provisional Application No. 61/227,716 filed on Jul. 22, 2009, each of which are hereby incorporated by reference in their entirety.

### FIELD OF THE INVENTION

The present invention relates to the utilization of dicarboxylic acid linked amino acid and peptide prodrugs of opioid analgesics, including oxycodone, codeine and dihydrocodeine, to treat pain, minimize the adverse gastrointestinal (GI) side-effects associated with the administration of the parent compound, and improve the respective opioid's pharmacokinetics.

### BACKGROUND OF THE INVENTION

Appropriate treatment of pain continues to represent a major challenge for both patients and healthcare professionals. Optimal pharmacologic management of pain requires selection of the appropriate analgesic drug that achieves rapid efficacy with minimal side effects. Full agonist opioid analgesics offer perhaps the most important option in the treatment of nociceptive pain and remain the gold standard of treatment. However, misuse and abuse of opioids is a widespread problem and may deter physicians from prescribing these drugs.

While affording good pain relief, opioids are blighted by unwanted GI side-effects, for example, constipation, nausea and vomiting. It has been found that a significant number of patients would rather endure their pain than suffer the incapacitating effects of chronic constipation, an enlightening measure of the severity and distress that this problem causes (Vanegas (1998). Cancer Nursing 21, 289-297).

A further shortcoming of many opioids is that they suffer from poor oral bioavailability. This has been shown, for example, with oxymorphone (Sloan et al. (2005). Supp Care Cancer 13, 57-65), meptazinol (Norbury et al. (1983). Eur J Clin Pharmacol 25, 77-80) and buprenorphine (Kintz and Marquet (2002). pp 1-11 in Buprenorphine Therapy in Opiate Addiction, Humana press). The poor oral bioavailability results in variable blood levels of the respective opioid, and therefore, variable patient response—a highly undesirable feature in the treatment of pain where rapid and reliable relief is demanded.

Additionally, opioid abuse is an increasing social problem. Amongst the opioids, oxycodone is one of the most widely abused drugs. Crushing and snorting the delayed release form, of oxycodone OxyContin®, results in rapid release of the drug, very rapid absorption, high peak serum concentrations, and can precipitate a fatal overdose (Aquina et al (2009) Post Graduate Medicine 121, 163-167). Necrosis of intranasal structures, similar to the damage associated with cocaine use has been reported as a result of prolonged OxyContin® abuse by snorting crushed tablets.

Various types of prodrugs have been proposed to improve the oral bioavailability of opioids. These have included simple ester conjugates which are frequently hydrolyzed by plasma esterases in a rapid fashion. Such hydrolysis by plasma esterases may limit the utility of ester linked prodrugs because it does not allow for transient protection of the opioid against first pass metabolism.

The rapidity of hydrolysis of ester conjugates is illustrated by work on the morphine ester prodrug morphine-3-propionate. Morphine has poor oral bioavailability due to extensive first pass glucuronidation at the 3 and the 6 positions, resulting in much inter and intra subject variability in analgesic response after an oral dose of the drug (Hoskin (1989). Br. J. Clin Pharmacol 27, 499-505). The plasma and tissue stability of the 3-propionate prodrug was investigated, and it was found to be hydrolyzed in human plasma with a half-life of less than 5 minutes (Goth et al. (1997). International Journal of Pharmaceutics 154, 149-155).

Meptazinol is another opioid with poor oral bioavailability (<10%). The low oral bioavailability has been attributed to high first pass glucuronidation (Norbury et al. (1983) Eur. J. Clin. Pharmacol. 25, 77-80). Attempts have been made to solve this problem by using ester linked meptazinol prodrugs (Lu et al. (2005). Biorg. and Med. Chem. Letters 15, 2607-2609 and Xie et al. (2005). Biorg. and Med. Chem. Letters 15, 493-4956). However, only one of these prodrugs -((Z)-3-[2-(propionyloxy)phenyl]-2-propanoic ester) showed a significant increase in bioavailability over meptazinol itself, when tested in a rat model.

A further issue with simple ester conjugates is their potential for chemical hydrolysis within the gut. For example, the valine ester of acyclovir undergoes some 15-25% chemical degradation in the GI tract before absorption (Granero and Amidon (2006). Internat. J. Pharmaceut. 317, 14-18.

More sophisticated ester conjugated opioid prodrugs have been synthesized. These include anthranilate and acetyl salicylates of nalbuphine and naloxone (Harrelson and Wong (1988). Xenobiotica 18, 1239-1247). However, in the 20 years since these ester conjugates were reported, no prodrug products based on the report have emerged, which suggests that this approach may not have been successful.

An alternative prodrug strategy is the formation of O-alkyl (alkyl ether) or aryl ether conjugates. However, such derivatives appear to be very resistant to hydrolysis and metabolic activation. This is illustrated by the 3-methyl ether prodrug of morphine-codeine. While codeine was not originally developed as a prodrug of morphine, it was subsequently found to give rise to small quantities of morphine. It has been estimated that less than 5% of an oral dose of codeine is converted to morphine—reflecting the slowness with which O-dealkylation takes place (Vree et al. (1992). Biopharma Drug Dispos. 13, 445-460 and Quiding et al. (1993). Eur. J. Clin. Pharmacol. 44, 319-323). The same phenomenon was observed for the corresponding dihydromorphine prodrug—dihydrocodeine, with less than 2% of an oral dose of dihydrocodeine being converted to dihydromorphine (Balikova et al. (2001). J. Chromatog. Biomed. Sci. Appl. 752, 179-186).

A further disadvantage of the O-alkyl ether prodrugging strategy is that the dealkylation of these opioids is effected by cytochrome P450 2D6 (Cyp2D6), a polymorphically expressed enzyme (Schmidt et al. (2003). Int. J. Clin. Pharmacol. Ther. 41, 95-106). This inevitably results in substantial variation in patient exposure to the respective active metabolite (e.g., morphine and dihydromorphine). Low exposure to morphine derived from codeine has been reported amongst a large group of patients deficient in Cyp2D6 activity, potentially impacting the analgesic efficacy of codeine (Poulsen et al. (1998). Eur. Clin. Pharmacol. 54, 451-454).

Additionally, a xenobiotic chemical prodrug moiety has the potential to contribute additional, additive or synergistic toxicities to those associated with the parent drug molecule.

An ideal prodrug moiety and linkage for a particular opioid would be cleaved at the appropriate rate and site, to form the active opioid compound. There remains a need in the treatment of severe pain with opioids, for products which retain all the inherent pharmacological advantages of the opioids, but which avoid their principal limitations of (1) induction of adverse GI side effects, including chronic constipation; and (2) low and erratic systemic availability after oral dosing.

### SUMMARY OF THE INVENTION

The present invention is directed to an opioid prodrug of Formula 1,

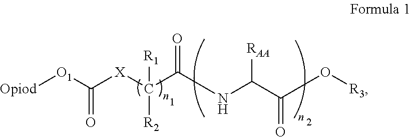

or a pharmaceutically acceptable salt thereof,

wherein,

O₁ is an oxygen atom present in the unbound opioid molecule;

X is (—NH—), (—O—), or absent;

each occurrence of R₁ and R₂ is independently selected from hydrogen, alkoxy,

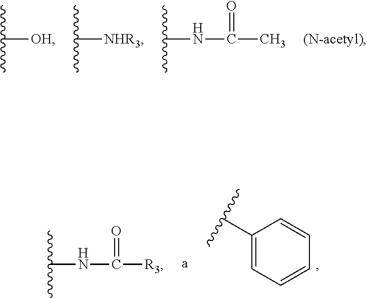

carboxyl, cycloalkyl, substituted cycloalkyl, alkyl and substituted alkyl;

R₁ and R₂ on adjacent carbons can form a ring and R₁ and R₂ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

in the case of a double bond in the carbon chain defined by n₁, R₁ is present and R₂ is absent on the carbons that form the double bond;

each occurrence of R₁ and R₂ can be the same or different;

R₃ is independently selected from hydrogen, alkyl, substituted alkyl, and an opioid;

when R₃ is an opioid, the —O— is a hydroxylic oxygen present in the additional opioid R₃;

each occurrence of R_{AA} is independently selected from a proteinogenic or non-proteinogenic amino acid side chain; and

the opioid is selected from any opioid with a hydroxyl, phenolic or carbonyl function, or an active metabolite thereof.

In one embodiment, the opioid is selected from butorphanol, buprenorphine, codeine, dezocine, dihydrocodeine, hydrocodone, hydromorphone, levorphanol, meptazinol, morphine, nalbuphine, oxycodone, oxymorphone, and pentazocine.

In a further embodiment, the opioid is an active metabolite of meptazinol selected from des-methyl meptazinol, 2-oxomeptazinol, 7-oxomeptazinol. ethyl-hydroxylated meptazinol (3-[3-(2-Hydroxy-ethyl)-1-methyl-perhydro-azepin-3-yl]-phenol), and ethyl-carboxylated meptazinol (3-[3-(2-carboxy-ethyl)-1-methyl-perhydro-azepin-3-yl]-phenol).

In yet another embodiment, the opioid is selected from naloxone and naltrexone.

In a further embodiment, n₁ is an integer selected from 0 to 4.

In one embodiment, X is absent, n₁ is 1 or 2 and n₂ is 1, 2, 3, 4 or 5. In one embodiment, n₂ is 1, 2 or 3. In a preferred embodiment, the prodrug moiety of the compound of Formula 1 has one or two amino acids (i.e., n₂ is 1 or 2).

In a preferred embodiment, X is absent, n₁ is 0, 1 or 2, n₂ is 1, 2 or 3 while R₃ is H. In another embodiment, n₂ is 1. In yet another embodiment, n₂ is 2. In yet another embodiment, n₂ is 1 or 2 and each occurrence of R_{AA} is independently a proteinogenic amino acid side chain. In yet another embodiment, n₁ is 1 or 2, n₂ is 1 or 2 and each occurrence of R_{AA} is independently a proteinogenic amino acid side chain.

In another embodiment, the present invention is directed to a pharmaceutical composition comprising one or more of the opioid prodrugs of the present invention, and one or more pharmaceutically acceptable excipients.

In a further embodiment, the methods, compounds and compositions of the present invention utilize conjugates of oxycodone, codeine or dihydrocodeine. The compounds can comprise from one to four amino acids, i.e., n₂ is 1, 2, 3 or 4. In a further embodiment, n₂ is either 1, 2 or 3. In a further embodiment, n₁ is 1 or 2 while n₂ is either 1, 2 or 3. In even a further embodiment, X is absent from Formula 1.

In one embodiment, X is absent from the

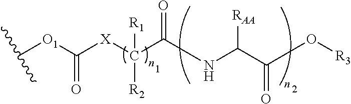

moiety, giving the
moiety

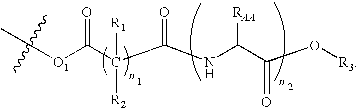

In a further embodiment, the

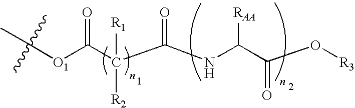

moiety of the present invention is selected from valine succinate, methionine succinate, 2-amino-butyric acid succinate, alanine succinate, phenylalanine succinate, isoleucine succinate, 2-amino acetic acid succinate, leucine succinate, alanine-alanine succinate, valine-valine succinate, tyrosine-glycine succinate, valine-tyrosine succinate, tyrosine-valine succinate and valine-glycine succinate. In this embodiment, R₁, R₂ and R₃ are each H, and n₁ is 2 (as defined above, for Formula I).

Yet another embodiment of the present invention is a method of treating a disorder in a subject in need thereof with an opioid. The method comprises orally administering a therapeutically effective amount (e.g., an analgesic effective amount) of an opioid prodrug of the present invention to the subject. The disorder may be one treatable with an opioid. For example, the disorder may be pain, such as neuropathic pain or nociceptive pain. Specific types of pain which can be treated with the opioid prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment.

In yet another embodiment, the present invention is directed to a method for minimizing the gastrointestinal side effects normally associated with administration of an opioid analgesic. Preferably, the opioid has a derivatizable group (e.g., a hydroxyl, phenolic or carbonyl group). The method comprises orally administering an opioid prodrug or a pharmaceutically acceptable salt thereof to a subject in need thereof, wherein the opioid prodrug is comprised of an opioid analgesic covalently bonded via a dicarboxylic acid linker, to an amino acid or peptide of 2-9 amino acids in length, and wherein upon oral administration, the prodrug or pharmaceutically acceptable salt minimizes, if not completely avoids, the gastrointestinal side effects usually seen after oral administration of the unbound opioid analgesic. The opioid prodrug may have the structure of Formula 1, or be a pharmaceutically acceptable salt thereof. The amount of the opioid is preferably a therapeutically effective amount (e.g., an analgesic effective amount).

In yet another embodiment, the present invention is directed to a method for reducing the intranasal abuse liability frequently associated with the use of opioid analgesis. Preferably, the opioid has a derivatizable group (e.g., a hydroxyl, phenolic or carbonyl group). The opioid prodrug or a pharmaceutically acceptable salt comprises an opioid analgesic covalently bonded via a dicarboxylic acid linker, to an amino acid or peptide of 2-9 amino acids in length, and whereupon illicit intranasal abuse, the prodrug or pharmaceutically acceptable salt is negligibly absorbed from nasal mucosa in comparison to the unbound opioid analgesic. The opioid prodrug may have the structure of Formula 1, or be a pharmaceutically acceptable salt thereof.

In yet a further embodiment, the present invention is directed to a method for reducing the intravenous abuse liability frequently associated with the use of opioid analgesis. Preferably, the opioid has a derivatizable group (e.g., a hydroxyl, phenolic or carbonyl group). The opioid prodrug or a pharmaceutically acceptable salt comprises an opioid analgesic covalently bonded via a dicarboxylic acid linker, to an amino acid or peptide of 2-9 amino acids in length, and where upon illicit intravenous use the prodrug or pharmaceutically acceptable salt results in slow attainment of reduced blood levels of the drug in comparison to the unbound opioid analgesic. The opioid prodrug may have the structure of Formula 1, or be a pharmaceutically acceptable salt thereof.

In another embodiment, the present invention is directed to a method for increasing the oral bioavailability of an opioid analgesic which has a significantly lower bioavailability when administered alone. Preferably, the opioid has a derivatizable group (e.g., a hydroxyl, phenolic or carbonyl group). The method comprises administering, to a subject in need thereof, an opioid prodrug or a pharmaceutically acceptable salt thereof, wherein the opioid prodrug is comprised of an opioid analgesic covalently bonded via a dicarboxylic acid linker, to an amino acid or peptide of 2-9 amino acids in length, and wherein upon oral administration, the oral bioavailability of the opioid derived from the prodrug is at least 20% greater than that of the opioid, when administered alone. The opioid prodrug may have the structure of Formula 1, or be a pharmaceutically acceptable salt thereof. The amount of the opioid is preferably a therapeutically effective amount (e.g., an analgesic effective amount).

In yet another embodiment, a method is provided for reducing the inter- or intra-subject variability of an opioid's plasma levels. The method comprises administering, to a subject in need thereof, or group of subjects in need thereof, an opioid prodrug or a pharmaceutically acceptable salt thereof, wherein the opioid prodrug is comprised of an opioid analgesic covalently bonded via a dicarboxylic acid linker, to an amino acid or peptide of 2-9 amino acids in length. The opioid prodrug may have the structure of Formula 1, or be a pharmaceutically acceptable salt thereof. The amount of the opioid is preferably a therapeutically effective amount (e.g., an analgesic effective amount).

The present invention relates to proteinogenic and/or non-proteinogenic amino acids and short-chain peptides of opioid analgesics which may also serve to sustain delivery a pharmacologically effective amount of the drug into the blood stream for the reduction or elimination of pain. The presence of quantities of unhydrolyzed prodrug in plasma provides a reservoir for continued generation of the active drug. This provides maintenance of plasma drug levels which reduces the frequency of drug dosage, and this would be expected to improve patient compliance. Additionally, avoidance of direct contact between the active drug and opioid receptors in the gut reduces the potential for adverse GI side effects commonly associated with opioid administration.

Another advantage of the prodrugs of the present invention resides in the possibility of sustaining plasma drug concentrations (from the continuing systemic generation of drug from prodrug) relative to the levels that would be present in the case that the opioid alone were to be administered. The consequences flowing from this might include the ability to use a reduced dosing frequency and/or improved patient compliance.

These and other embodiments are disclosed or are apparent from and encompassed by the following Detailed Description.

### BRIEF DESCRIPTION OF THE DRAWINGS

FIG. 1 shows the oxycodone plasma concentration vs. time profile in dogs after oral administration of either oxycodone itself (1 mg free base/kg) or oxycodone succinyl valine ester (1 mg free base oxycodone equivalents/kg).

FIG. 2 shows the codeine plasma concentration vs. time profile in dogs after oral administration of either codeine itself (1 mg free base/kg) or codeine succinyl valine ester (1 mg free base codeine equivalents/kg).

FIG. 3 shows the dihydrocodeine plasma concentration vs. time profile in dogs after oral administration of either dihydrocodeine itself (1 mg free base/kg) or dihydrocodeine succinyl valine ester (1 mg free base dihydrocodeine equivalents/kg).

FIG. 4 illustrates the relationship between the log concentration of oxycodone or oxycodone succinyl valine ester (expressed as the free base of oxycodone) addition to isolated guinea pig ileum preparations and the effects on electrical field stimulation response.

FIG. 5 illustrates the relationship between the log concentration of codeine or codeine succinyl valine ester (expressed as the free base of codeine) after addition to isolated guinea pig ileum preparations and the effects on electrical field stimulation response.

FIG. 6 illustrates the relationship between the log concentration of dihydrocodeine or dihydrocodeine succinyl valine ester (expressed as the free base of dihydrocodeine) after addition to isolated guinea pig ileum preparations and the effects on electrical field stimulation response.

FIG. 7 shows the oxycodone plasma concentration vs. time profile in the male cynomolgus monkey after oral administration of either oxycodone itself (1 mg/kg) or oxycodone succinyl valine enol ester (OSVE; 1 mg free base oxycodone equivalent/kg)

FIG. 8 shows the oxycodone plasma concentration vs time profile in the male cynomolgus monkey after oral administration of either oxycodone itself (1 mg/kg) or oxycodone glutaryl leucine enol ester (OGLE; 1 mg free base oxycodone equivalent/kg).

FIG. 9 shows the oxycodone plasma concentration vs. time profile in female rats after oral administration of oxycodone hydrochloride (10 mg free base equivalents/kg).

FIG. 10 shows the oxycodone plasma concentrations vs. time profile in female rats following oral administration of oxycodone [succinyl-(S)-valine] enol ester TFA (10 mg oxycodone free base equivalents/kg).

FIG. 11 shows the oxycodone plasma concentration vs. time profile in dogs after administration by intranasal insufflation of oxycodone HCl (˜0.25 mg oxycodone free base equivalents/kg).

FIG. 12 shows the oxycodone plasma concentrations after administration by intranasal insufflation of oxycodone [succinyl-(S)-valine] enol ester TFA to dogs (0.25 mg oxycodone free base equivalents/kg).

### DETAILED DESCRIPTION OF THE INVENTION

### Definitions

As used herein:

The term “peptide” refers to an amino acid chain consisting of 2 to 9 amino acids, unless otherwise specified. In a preferred embodiment, the peptide used in the present invention is 2 or 3 amino acids in length. In one embodiment, a peptide can be a branched peptide. In this embodiment, at least one amino acid side chain in the peptide is bound to another amino acid (either through one of the termini or the side chain).

The term “amino acid” refers both to proteinogenic and non-proteinogenic amino acids. The amino acids contemplated for use in the prodrugs of the present invention include both proteinogenic and non-proteinogenic amino acids, preferably proteinogenic amino acids. The side chains R_{AA} can be in either the (R) or the (S) configuration. Additionally, both D and L amino acids are contemplated for use in the present invention.

A “proteinogenic amino acid” is one of the twenty two amino acids used for protein biosynthesis as well as other amino acids which can be incorporated into proteins during translation (including pyrrolysine and selenocysteine). A proteinogenic amino acid generally has the formula

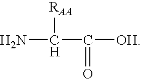

R_{AA} is referred to as the amino acid side chain, or in the case of a proteinogenic amino acid, as the proteinogenic amino acid side chain. The proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine, histidine, selenocysteine and pyrrolysine. Another term for a “proteinogenic amino acid” is a “natural amino acid.”

Examples of proteinogenic amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

In one embodiment, an amino acid side chain is bound to another amino acid. In a further embodiment, the side chain is bound to the amino acid via the amino acid's N-terminus, C-terminus, or side chain.

A “non-proteinogenic amino acid” is an organic compound that is not among those encoded by the standard genetic code, or incorporated into proteins during translation. Non-proteinogenic amino acids, thus, include amino acids or analogs of amino acids other than the 22 proteinogenic amino acids used for protein biosynthesis and include, but are not limited to, the D-isostereomers of proteinogenic amino acids. Additionally, amino acids are included in the definition on “non-proteinogenic amino acids.” Another term for a “non-proteinogenic amino acid” is a “non-natural amino acid.”

Examples of non-proteinogenic amino acids include, but are not limited to: citrulline, homocitrulline, hydroxyproline, homoarginine, homoserine, homotyrosine, homoproline, ornithine, 4-amino-phenylalanine, 4-nitro-phenylalanine, 4-fluoro-phenylalanine, 2,3,4,5,6-pentafluoro-amino-phenylalanine, sarcosine, biphenylalanine, homophenylalanine, norleucine, cyclohexylalanine, α-aminoisobutyric acid, acedic acid, N-acetic acid, O-methyl serine (i.e., an amino acid sidechain having the formula

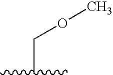

), N-methyl-alanine, N-methyl-glycine, N-methyl-glutamic acid, tert-butylglycine, α-aminobutyric acid, 2-aminoisobutyric acid, 2-aminoindane-2-carboxylic acid, selenomethionine, lanthionine, diethylglycine, dipropylglycine, cyclohexylglycine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, α-aminoisobutyric acid, acetylamino alanine (i.e., an amino acid sidechain having the formula

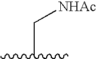

), β-alanine, β-(acetylamino)alanine, β-aminoalanine, β-chloroalanine, phenylglycine, dehydroalanine, and derivatives thereof wherein the amine nitrogen has been mono- or di-alkylated.

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The term “amino” refers to a —NH₂ group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, carboxyl alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, carboxyl, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The terms “keto” and “oxo” are synonymous, and refer to the group ═O.

The term “carbonyl” refers to a group —C(═O).

The term “carboxyl” refers to a group —CO₂H and consists of a carbonyl and a hydroxyl group (More specifically, C(═O)OH).

The terms “dicarboxylic acid linker” and “dicarboxyl linker,” for the purposes of the present invention, are synonymous. The dicarboxylic acid linker refers to the group between the opioid and the amino acid/peptide moiety:

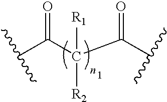

(—(CO)—(CR₁R₂)ₙ₁—(CO)—).

Alternatively, the “dicarboxylic acid linker” can have the formula:

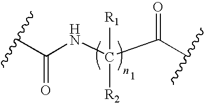

(—(CO)—(NH)—(CR₁R₂)ₙ₁—(CO)—), or the formula:

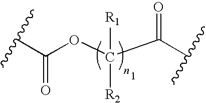

(—(CO)—(O)—(CR₁R₂)ₙ₁—(CO)—).

Regarding the dicarboxylic acid linker, one carbonyl group is bound to an oxygen atom in the opioid, while the second carbonyl is bound to the N terminus of a peptide or amino acid, or an amino group of an amino acid side chain.

Prodrug moieties described herein may be referred to based on their amino acid or peptide and the dicarboxyl linkage. The amino acid or peptide in such a reference should be assumed to be bound via an amino terminus on the amino acid or peptide to one carbonyl (originally part of a carboxyl group) of the dicarboxyl linker while the other is attached to the opioid analgesic, unless otherwise specified. The dicarboxyl linker may or may not be variously substituted as stipulated earlier.

A non-limiting list of dicarboxylic acids for use with the present invention are given in Tables 1 and 2. Although the dicarboxylic acids listed in Table 1 contain from 2 to 18 carbons, longer chain dicarboxylic acids can be used as linkers in the present invention. Additionally, the dicarboxylic acid linker can be substituted at one or more positions (see Table 2). A dicarboxylic acid, suitably activated, can be combined with an activated amino acid or peptide, and then reacted with an opioid, to form a prodrug of the present invention. Procedures for synthesizing these prodrugs are discussed in more detail in the example section.

TABLE 1
Examples of Dicarboxylic Acids For Use With The Present Invention

| Common Name | IUPAC Name | Chemical Formula |
|---|---|---|
| Oxalic Acid | Ethanedioic Acid | HOOC—COOH |
| Malonic Acid | Propanedioic Acid | HOOC—(CH₂)—COOH |
| Succinic Acid | Butanedioic Acid | HOOC—(CH₂)₂—COOH |
| Glutaric Acid | Pentanedioic Acid | HOOC—(CH₂)₃—COOH |
| Adipic Acid | Hexanedioic Acid | HOOC—(CH₂)₄—COOH |
| Pimelic Acid | Heptanedioic Acid | HOOC—(CH₂)₅—COOH |
| Suberic Acid | Octanedioic Acid | HOOC—(CH₂)₆—COOH |
| Azelaic Acid | Nonanedioic Acid | HOOC—(CH₂)₇—COOH |
| Sebacic Acid | Decanedioic Acid | HOOC—(CH₂)₈—COOH |
| Undecanedioic Acid | Undecanedioic Acid | HOOC—(CH₂)₉—COOH |
| Dodecanedioic Acid | Dodecanedioic Acid | HOOC—(CH₂)₁₀—COOH |
| Brassylic Acid | Tridecanedioic Acid | HOOC—(CH₂)₁₁—COOH |
| | 1,11-Undecanedicarboxylic Acid | |
| Tetradecanedioic Acid | 1,12-Dodecanedicarboxylic Acid | HOOC—(CH₂)₁₂—COOH |
| Pentadecanedioic Acid | 1,15-Pentadecanedioic Acid | HOOC—(CH₂)₁₃—COOH |
| Thapsic Acid | Hexadecanedioic Acid | HOOC—(CH₂)₁₄—COOH |
| | Hexane-1,16-dioic Acid | |
| Heptadecanedioic Acid | 1,15-Pentadecanedicarboxylic Acid | HOOC—(CH₂)₁₅—COOH |
| Octadecanedioic Acid | 1,16-Tetradecanedicarboxylic Acid | HOOC—(CH₂)₁₆—COOH |
| Phthalic Acid | Benzene-1,2-Dicarboxylic Acid | C₆H₄(COOH)₂ |
| Terephthalic Acid | Benzene-1,4-Dicarboxylic Acid | C₆H₄(COOH)₂ |
| Aconitic Acid | Prop-1-ene-1,2,3-tricarboxylic acid | C₆H₆O₆ |
| Achilleic Acid | | |
| Citraconic Acid | 2-methylbut-2-enedioic acid | C₅H₆O₄ |
| Itaconic Acid | Methylenesuccinic Acid | C₅H₆O₄ |
| | 2-Methylidenebutanedioic acid | |
| Aconitic Acid | Prop-1-ene-1,2,3-tricarboxylic acid | C₆H₆O₆ |
| α-Ketoglutaric Acid | 2-oxopentanedioic acid | C₅H₆O₅ |
| N^{α}-Acetyl glutamatic | 2-acetamidopentanedioic acid | C₇H₁₁NO₅ |
| acid | | |
| Isocitric acid | 1-Hydroxypropane-1,2,3-tricarboxylic | C₆H₈O₇ |
| | acid | |
| 2-hydroxy-3-methylsuccinic | 2-hydroxy-3-methylsuccinic acid | C₅H₉O₅ |
| acid | | |
| 2-hydroxy-2,3-dimethyl | 2-hydroxy-2,3-dimethylsuccinic | C₆H₁₀O₅ |
| succinic acid | acid | |
| citric acid | 2-hydroxypropane-1,2,3-tricarboxylic | C₆H₈O₇ |
| | acid | |

Dicarboxylic acid linkers of the present invention can have a nitrogen or oxygen atom bound to the first carbonyl group, i.e., X is (—NH—) or (—O—) in Formula 1, to give the linker structures

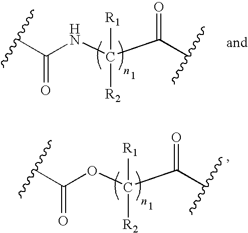

respectively. Examples of such dicarboxylic acid linkers are given in Table 2, below and throughout the specification.

In one embodiment, the dicarboxylic acid linker is substituted. For example, one or more alkoxy,

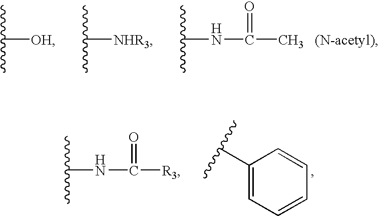

carboxyl, cycloalkyl, substituted cycloalkyl, alkyl and substituted alkyl may be present (R₁, R₂, and R₃, as defined by Formula 1). In these embodiments, X (—NH— or —O—, as defined by Formula 1) may be present or absent. Examples of dicarboxylic acid linkers are given in Table 2.

In one embodiment, the carbon chain

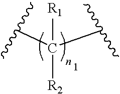

in the dicarboxylic acid linker is unsaturated, and can have one or more double bonds (e.g., maleic acid, fumaric acid, or citraconic acid linker). In these embodiments, n₁≧2 and R₂ is absent on the two carbons that form the double bond (e.g., fumaric acid, see Table 2). Table 2 is directed to various dicarboxylic acid linkers of the present invention. The broken lines in the second column of Table 2 indicate where an opioid, amino acid or peptide can be bound to the respective dicarboxylic acid linker. The definition of R₃ is provided by Formula 1 (see supra). Although not depicted in Table 2, the linkers with an additional carboxylic acid (e.g., the citric acid linkers) can have an amino acid or peptide bound thereto.

TABLE 2.
Non-Limiting List of Dicarboxylic Acid Linkers For Use With The Present Invention

| Dicarboxylic Acid | | Valine Prodrug Moiety (opioid |
|---|---|---|
| Linker Name | Structure | hydroxylic oxygen shown as O1) |
| N^{α}-Acetyl Aspartic Acid Linker | 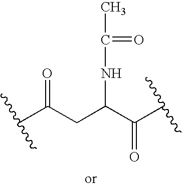 | 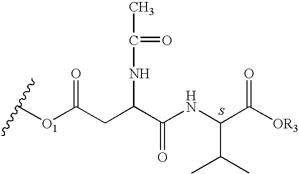 |
| | 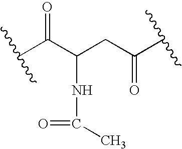 | |
| N^{α}-Acetyl Glutamic Acid Linker | 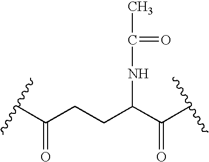 | 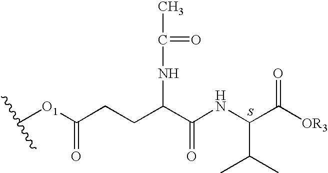 |
| Malic Acid Linker | 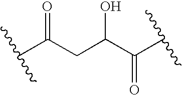 | 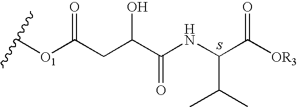 |
| Tartaric Acid Linker | 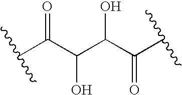 | 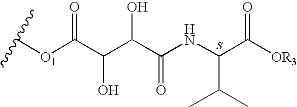 |
| Citramilic Acid Linker | 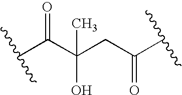 | 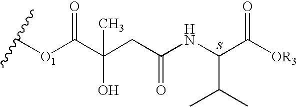 |
| 2-Methyl Succinic Acid Linker | 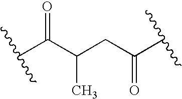 | 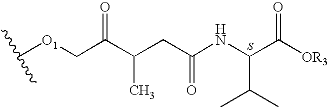 |
| 2,2-Dimethyl Succinic Acid Linker | 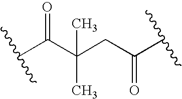 | 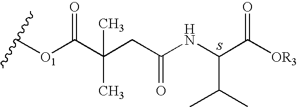 |
| 2,3-Dimethyl Succinic Acid Linker | 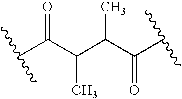 | 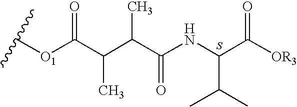 |
| (S)-Citramalic Acid Linker | 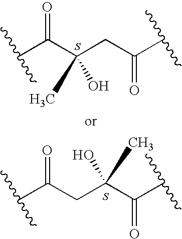 | 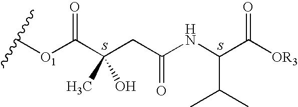 |
| 2-Phenylsuccinic Acid Linker | 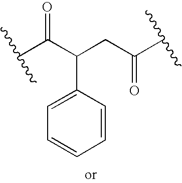 | 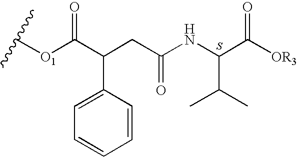 |
| | 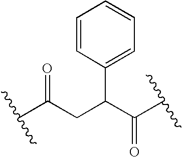 | |
| 2,2-Dimethylglutaric Acid Linker | 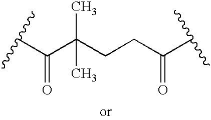 | 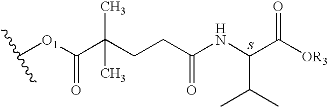 |
| | 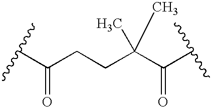 | |
| 3,3-Dimethylglutaric Acid Linker | 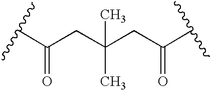 | 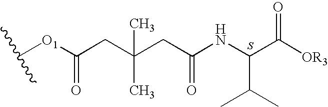 |
| β-Alanine Linker | 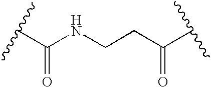 | 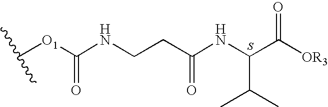 |
| γ-Aminobutyric Acid (GABA) Linker | 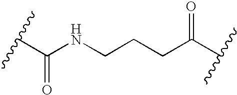 | 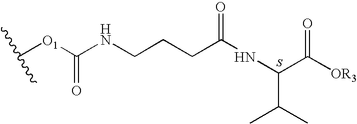 |
| 3-(Carboxyoxy) Butanoic Acid Linker | 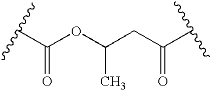 | 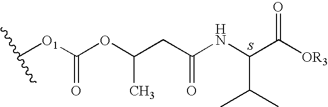 |
| 3-(Carboxyoxy) Propanoic Acid Linker | 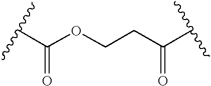 | 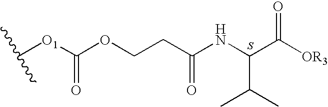 |
| 4-(Carboxyoxy) Butanoic Acid Linker | 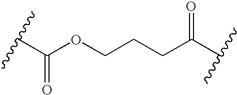 | 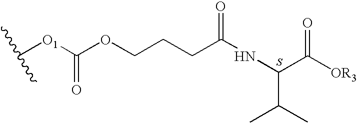 |
| Glutaconic Acid Linker | 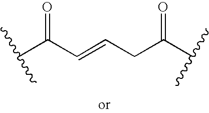 | 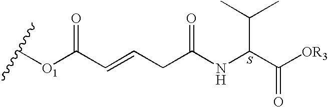 |
| | 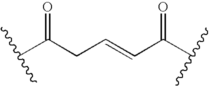 | |
| Ketoglutaric Acid Linker | 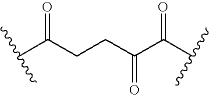 | 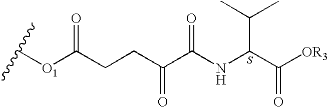 |
| Maleic Acid Linker | 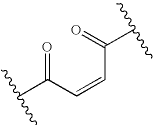 | 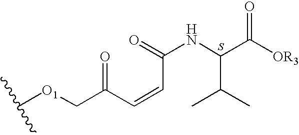 |
| Citraconic Acid Linker | 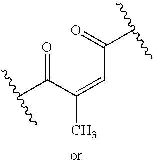 | 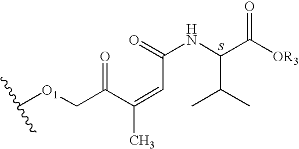 |
| | 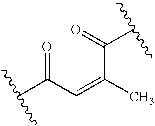 | |
| 2,3-Dimethylmaleic Acid Linker | 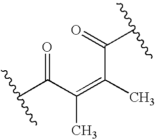 | 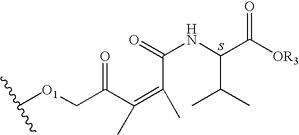 |
| Fumaric Acid Linker | 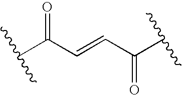 | 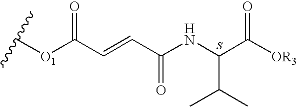 |
| 2,3-Dimethylfumaric Acid Linker | 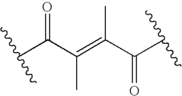 | 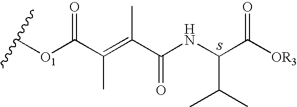 |
| Z-Methoxybutenedioic Acid Linker | 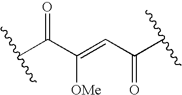 | 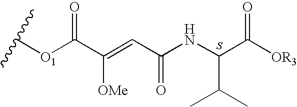 |
| Aconitic Acid Linker | 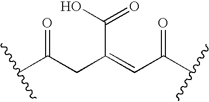 | 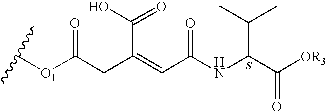 |
| E-Methoxybutenedioc Acid Linker | 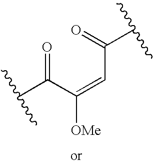 | 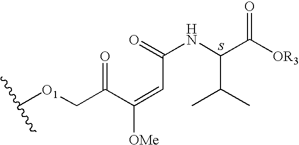 |
| | 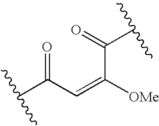 | |
| 2-Methylene Glutaric Acid Linker | 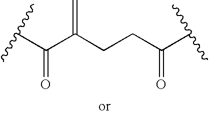 | 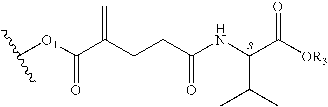 |
| | 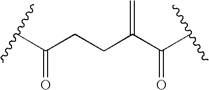 | |
| Itaconic Acid Linker | 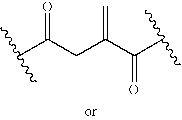 | 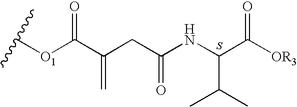 |
| | 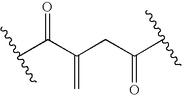 | |
| Terephtthalic Acid Linker | 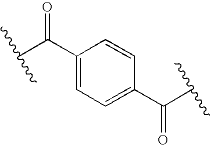 | 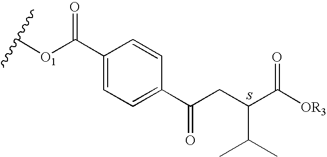 |
| Phthalic Acid Linker | 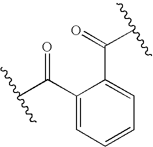 | 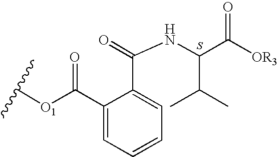 |
| Citroyl Acid Linker | 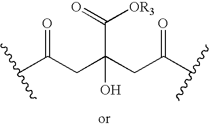 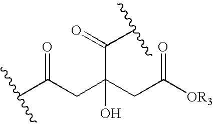 | 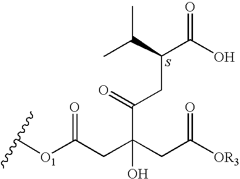 |
| Citric Acid Linker (1) | 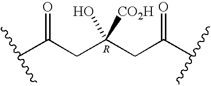 | 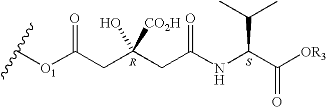 |
| Citric Acid Linker (2) | 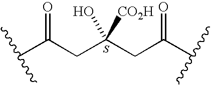 | 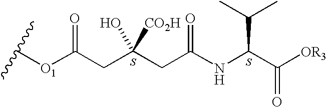 |
| Citric Acid Linker (3) | 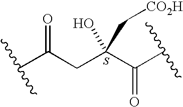 | 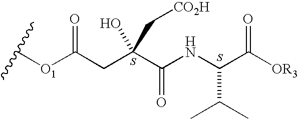 |
| Citric Acid Linker (4) | 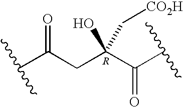 | 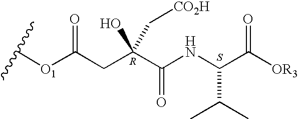 |
| Citric Acid Linker (5) | 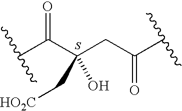 | 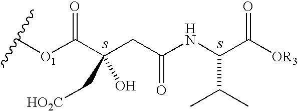 |
| Citric Acid Linker (6) | 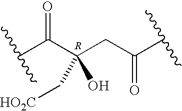 | 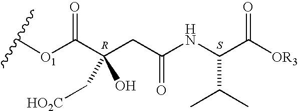 |

Examples of prodrug moieties of the present invention include valine succinate, which has the formula

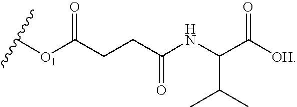

For a dipeptide, such as tyrosine-valine succinate, it should be assumed unless otherwise specified that the amino acid adjacent to the drug, in this case valine, is attached via the amino terminus to the dicarboxylic acid linker. The terminal carboxyl residue of the dipeptide (in this case tyrosine) forms the C (carboxyl) terminus.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be analgesia and/or an amelioration of one or more gastrointestinal side effect symptoms that are present when the respective opioid in the prodrug is administered in its active form (i.e., when the opioid is administered alone). It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to the opioid portion of a prodrug of the present invention, as described herein.

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66:1).

The term “bioavailability,” as used herein, generally means the rate and/or extent to which the active ingredient is absorbed from a drug product and becomes systemically available, and hence available at the site of action. See Code of Federal Regulations, Title 21, Part 320.1 (2003 ed.). For oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of a respective opioid given orally that is absorbed into the systemic circulation after a single administration to a subject. A preferred method for determining the oral bioavailability is by dividing the AUC of the opioid given orally by the AUC of the same opioid dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

The term “increase in oral bioavailability” refers to the increase in the bioavailability of a respective opioid when orally administered as a prodrug of the present invention (either a prodrug compound or composition), as compared to the bioavailability when the opioid is orally administered alone. The increase in oral bioavailability can be from 5% to 20,000%, 10% to 10,000%, preferably from 200% to 20,000%, more preferably from 500% to 20,000%, and most preferably from 1000% to 20,000%. The increase in oral bioavailability can be by at least 10%, at least 20%, at least 30%, at least 40%, at least 50%, at least 60%, at least 70%, at least 80%, or at least 90%.

The term “low oral bioavailability,” refers to an oral bioavailability wherein the fraction of a dose of the parent drug given orally that is absorbed into the plasma unchanged after a single administration to a subject is 25% or less, preferably 15% or less, and most preferably 10% or less. Without wishing to be bound by any particular theory, it is believed that the low oral bioavailability of the opioids described herein is the result of the conjugation of a phenolic or -hydroxylic oxygen to glucuronic acid during first pass metabolism. However, other mechanisms may be responsible for the decrease in oral bioavailability and are contemplated by the present invention.

It will also be appreciated by a person skilled in the art that the compounds of the invention could be made by adaptation of the methods herein described and/or adaptation of methods known in the art, for example the art described herein, or using standard textbooks such as “Comprehensive Organic Transformations—A Guide to Functional Group Transformations”, R C Larock, Wiley-VCH (1999 or later editions), “March's Advanced Organic Chemistry—Reactions, Mechanisms and Structure”, M B Smith, J. March, Wiley, (5th edition or later) “Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook To Organic Synthesis” R K Mackie and D M Smith (Longman) (1982 or later editions), etc., and the references therein as a guide.

It will also be apparent to a person skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (tautomerism) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula I, including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and a ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the Invention

In some embodiments the substituents on the alkylene carbon in the general formula 1, 2, 3 etc are R₁ and R₂ and in other embodiments the substituents on the alkylene carbon are R₃ and R₄.

In some embodiments the terminal ester group in the general formula 1, 2, 3 etc is defined by R₃ and in other embodiments the terminal ester group is defined by R₅.

The reader will appreciate to which embodiments the various substituents designations apply. However, it is intended that the corresponding substituent groups on the alkylene, and at the terminus respectively, should have the same meaning

The prodrugs of the present invention are novel amino acid and peptide prodrugs of the opioids, wherein the opioid is bonded to the amino acid or peptide by a dicarboxylic acid linker group. Preferably, these prodrugs comprise the opioid attached to a single amino acid or short peptide through a dicarboxylic acid linker, wherein one carbonyl group of the linker is bound to either an opioid hydroxyl function, an opioid phenolic function, or an enolized keto function.

An —OH (hydroxyl) group can be esterified with a dicarboxylic acid such as, but not limited to, malonic, succinic, glutaric, adipic or other longer chain dicarboxylic acid, or substituted derivative thereof (for example, see Tables 1 and 2). In addition, a keto group can be enolized and then esterifed with a dicarboxylic acid such as the ones described above. The amino acid or peptide may then be attached to the remaining carboxyl group via the N-terminal nitrogen on the peptide/amino acid, or a nitrogen present in an amino acid side chain (e.g., a lysine side chain).

In one embodiment, the present invention is directed to an opioid prodrug of Formula 1,

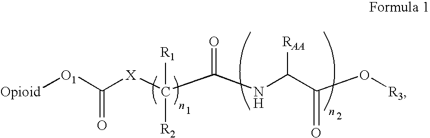

or a pharmaceutically acceptable salt thereof,

wherein,

O₁ is an oxygen atom present in the unbound opioid molecule;

X is (—NH—), (—O—), or absent;

Each occurrence of R₁ and R₂ is independently selected from hydrogen, alkoxy

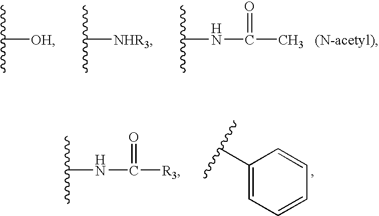

carboxyl, cycloalkyl, substituted cycloalkyl, alkyl, and a substituted alkyl;

R₁ and R₂ on adjacent carbons can form a ring and R₁ and R₂ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

in the case of a double bond in the carbon chain defined by n₁, R₁ is present and R₂ is absent on the carbons that form the double bond;

R₃ is independently selected from hydrogen, alkyl, substituted alkyl and an opioid;

When R₃ is an opioid, the —O— is a hydroxylic oxygen present in the additional opioid R₃;

Each occurrence of R_{AA} is independently selected from a proteinogenic or non-proteinogenic amino acid side chain; and

the opioid is selected from any opioid with a hydroxyl, phenolic or carbonyl function, or an active metabolite thereof.

In a further embodiment of Formula 1, n₁ is an integer selected from 0 to 4.

In yet a further embodiment, n₂ is 1 or 2, R₁, R₂ and R₃ are each hydrogen and n₁ is an integer selected from 0 to 4.

In one embodiment, the opioid is selected from butorphanol, buprenorphine, codeine, dezocine, dihydrocodeine, hydrocodone, hydromorphone, levorphanol, meptazinol, morphine, nalbuphine, oxycodone, oxymorphone, and pentazocine. In a further embodiment, n₁ is an integer selected from 0 to 4.

In another embodiment, the opioid is an opioid antagonist.

In a further embodiment, the opioid antagonist is selected from naloxone and naltrexone. In a further embodiment, n₁ is an integer selected from 0 to 4.

In one embodiment, X is absent, n₁ is 0, 1 or 2 and n₂ is 1, 2, 3, 4 or 5. In one embodiment, n₂ is 1, 2 or 3. In a preferred embodiment, the prodrug moiety of the compound of Formula 1 has one or two amino acids (i.e., n₂ is 2).

In a preferred embodiment, X is absent, n₁ is 1 or 2, n₂ is 1, 2 or 3 while R₃ is H. In another embodiment, n₂ is 1. In yet another embodiment, n₂ is 2. In yet another embodiment, n₂ is 1 or 2 and each occurrence of R_{AA} is independently a proteinogenic amino acid side chain.

In another embodiment, n₁ is 2.

In one embodiment, X is —O—, n₁ is 0, 1 or 2, n₂ is 1 or 2 and R₃ is H.

In another embodiment, X is —O—, n₁ is 1, 2, 3 or 4, n₂ is 1, 2 or 3 and R₃ is H. In a further embodiment, at least one occurrence of R₁ is methyl.

In one embodiment, X is —NH—, n₁ is 0, 1 or 2, n₂ is 1 or 2 and R₃ is H.

In another embodiment, X is —NH—, n₁ is 1, 2, 3 or 4, n₂ is 1, 2 or 3 and R₃ is H. In a further embodiment, at least one occurrence of R₁ is methyl.

In yet another embodiment, X is absent, n₁ is 2, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one embodiment, X is absent, n₁ is 2, and one occurrence of R₁ or R₂ is —CH₃. In a further embodiment, R₃ is hydrogen.

In yet another embodiment, X is absent, n₁ is 3, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one embodiment, X is absent, n₁ is 2, and one occurrence of R₁ or R₂ is

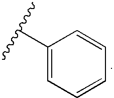

In a further embodiment, R₃ is hydrogen.

Another embodiment is directed to opioid prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₃ is hydrogen. In even a further embodiment, the proteinogenic amino acid side chain is selected from valine, leucine and isoleucine.

Yet another embodiment is directed to opioid prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₃ is hydrogen.

Itaconic acid, ketoglutaric and 2-methylene glutaric acid can also be used as a dicarboxylic acid linker in some embodiments. Here, R₁ and R₂ on one of the carbons defined by n₁, taken together, is a methylene group.

In one embodiment, the opioid prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another embodiment includes opioid prodrugs linked to a peptide or amino acid through a dicarboxylic acid linker substituted with an acetyl

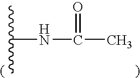

group or a carboxylic acid group. In a further embodiment, n₁ is 2 or 3 and R₃ is hydrogen. In even a further embodiment, the dicarboxylic acid linker is further substituted with an

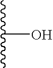

group.

In one embodiment, the opioid is linked to a peptide or prodrug through a citric acid linker. The citric acid linker can be any one of 6 isomers, as provided herein in Table 2.

In one embodiment, the opioid prodrug of the present invention uses a dicarboxylic acid disclosed in Table 1 or 2 as the dicarboxylic acid linker.

In another embodiment, R₃ is an opioid, and the two opioids are linked via citroyl acid linker. In this embodiment, the additional carboxylic acid in the citroyl acid linker is bound to an amino acid or peptide.

Preferred prodrug moieties of the present invention are when X is absent (i.e., the

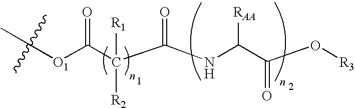

moiety, using the definitions provided for Formula 1). Examples of single amino acid prodrug moieties include valine succinate, leucine succinate and isoleucine succinate. Dipeptide moieties that are preferred include valine-valine succinate, leucine-leucine succinate and isoleucine-isoleucine succinate. In these embodiments, X is absent, R₁, R₂ and R₃ are H and n₁ is 2.

Peptides comprising any of the proteinogenic amino acids, as well as non-proteinogenic amino acids, can be used in the present invention. Examples of non-proteinogenic amino acids are given above. Non-proteinogenic amino acids can be present in a peptide with only non-proteinogenic amino acids, or alternatively, with both proteinogenic and non-proteinogenic amino acids.

The 22 proteinogenic amino acids used for protein biosynthesis, as well as their abbreviations, are given in Table 3 below.

TABLE 3
Proteinogenic Amino Acids (Used
For Protein Biosynthesis) and Their
Abbreviations

| | 3 letter | 1-letter |
|---|---|---|
| Amino acid | code | code |
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |
| Selenocysteine | SEC | U |
| Pyrrolysine | PYL | O |

The amino acids employed in the opioid prodrugs for use with the present invention are preferably in the L configuration. The present invention also contemplates prodrugs of the invention comprised of amino acids in the D configuration, or mixtures of amino acids in the D and L configurations.

In one embodiment, the peptide/amino acid (or multiple peptides or amino acids) can be bound to one of two (or both) possible locations in the opioid molecule. For example, morphine and dihydromorphine have hydroxyl groups at carbon 3 and carbon 6. A peptide or amino acid can be bound at either, or both of these positions. In this embodiment, each occurrence of n₁, n₂, R₁, R₂, R₃ and R_{AA} can be the same or different. In addition, X (—NH— or —O—) can be present in one moiety, while absent in the other, present in both, or absent in both moieties. Dicarboxylic acid linkages can be formed at either site, and upon peptide cleavage, the opioid will revert back to its original form.

When a ketone is present in the opioid scaffold (e.g., the ketone at the 6 position of hydromorphone and oxycodone), as stated above, the ketone can be converted to its corresponding enolate and reacted with a modified peptide reactant (which can be a modified amino acid) to form a prodrug. Upon peptide cleavage, the prodrug will revert back to the original opioid molecule, with the keto group present.

In a preferred embodiment, the dicarboxylic acid linker is succinic acid. Other dicarboxylic acid linkers within the scope of the invention include, but are not limited to, malonic acid, glutaric acid, adipic acid, or other longer chain dicarboxylic acids or substituted derivatives thereof (see Tables 1 and 2).

As alternatives to the use of an unsubstituted dicarboxylic acid linker to attach the opioid to the amino acid or peptide prodrug moiety, substituted dicarboxylic acid linkers may be employed. For example, methyl malonic acid may be used. Such substituted dicarboxylic acid linkers would preferably be naturally occurring in the subject to be treated, i.e., non-xenobiotic. Suitable substituted dicarboxylic acids are given in Table 2.

### Oxycodone Prodrugs of the Present Invention

In one embodiment, the prodrugs of the present invention are directed to oxycodone prodrugs of Formula 2, below.

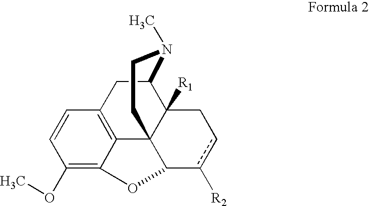

or a pharmaceutically acceptable salt thereof,

wherein,

R₁ is independently selected from

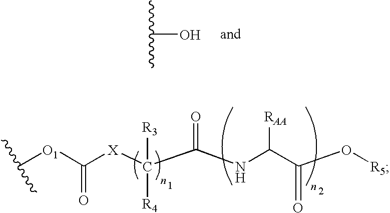

R₂ is selected from

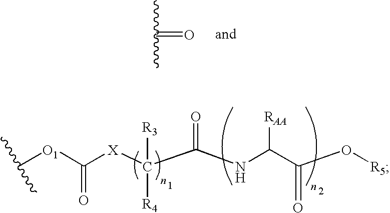

Each occurrence of O₁ is independently an oxygen atom in the unbound form of oxycodone;

Each occurrence of X is independently (—NH—), (—O—), or absent;

Each occurrence of R₃ and R₄ is independently selected from hydrogen, alkoxy,

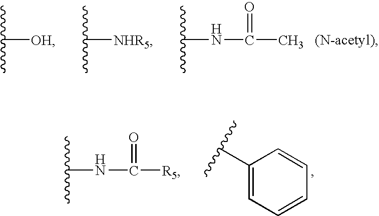

carboxyl, cycloalkyl, substituted cycloalkyl, alkyl, and substituted alkyl;

R₃ and R₄ on adjacent carbons can form a ring and R₃ and R₄ on the same carbon, taken together, can be a methylene group;

Each occurrence of n₁ is independently an integer selected from 0 to 16 and each occurrence of n₂ is independently an integer selected from 1 to 9, and each occurrence of n₁ and n₂ can be the same or different;

the carbon chain defined by n₁ can include a cycloalkyl or aromatic ring;

In the case of a double bond in the carbon chain defined by n₁, R₃ is present and R₄ is absent on the carbons that form the double bond;

Each occurrence of R₅ is independently selected from hydrogen, alkyl, substituted alkyl group and an opioid;

When R₅ is an opioid, the —O— is a hydroxylic oxygen present in the additional opioid R₅;

Each occurrence of R_{AA} is independently selected from a proteinogenic or non-proteinogenic amino acid side chain;

the dashed line in Formula 2 is absent when R₂ is

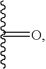

and a bond when R₂ is not

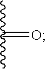

and

at least one of R₁ or R₂is

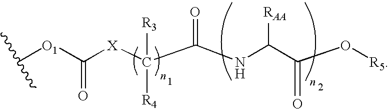

In a further Formula 2 embodiment, n₁ is an integer selected from 0 to 4.

In yet a further Formula 2 embodiment, R₂ is

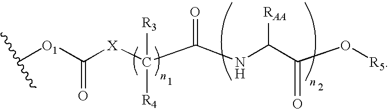

In even a further embodiment, X is absent and n₁ is 1, 2 or 3.

In one embodiment, R₁ is

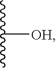

X is absent, n₁ is 0, 1, 2 or 3, n₂ is 1, 2 or 3 and R₃, R₄ and R₅ are each H. In a further embodiment, n₁ is 2.

In one embodiment, R₂ is

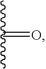

X is absent, n₁ is 0, 1, 2 or 3, n₂ is 1, 2 or 3 and R₃, R₄ and R₅ are each H. In a further embodiment, n₁ is 2.

In one embodiment, R₁ is

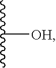

X is absent, n₁ is 0, 1, 2 or 3, n₂ is 1, 2, 3, 4 or 5 and R₃, R₄ and R₅ are each H. In a further embodiment, n₁ is 2.

In one embodiment, R₂ is

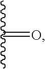

X is absent, n₁ is 0, 1, 2 or 3, n₂ is 1, 2, 3, 4 or 5 and R₃, R₄ and R₅ are each H. In a further embodiment, n₁ is 2.

In one embodiment, X is —O—, n₁ is 0, 1 or 2, n₂ is 1 or 2 and R₅ is H. In a further embodiment, n₁ is 2 and R₁ is

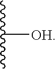

In one embodiment, X is —NH—, n₁ is 0, 1 or 2, n₂ is 1 or 2 and R₅ is H. In a further embodiment, n₁ is 2 and R₁ is

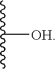

In a preferred embodiment, the oxycodone prodrug of the present invention has one prodrug moiety, and the prodrug moiety has one or two amino acids (i.e., n₂ is 1 or 2). In one embodiment, the oxycodone prodrug of the present invention has one prodrug moiety, and n₁ is 1 or 2 while n₂ is 1, 2 or 3 and R₅ is H.

In a preferred embodiment, n₂ is 1, 2 or 3 while R₃, R₄ and R₅ are H. In another embodiment, n₂ is 1. In yet another embodiment, n₂ is 2. In yet another embodiment, n₂ is 1 or 2 and each occurrence of R_{AA} is independently a proteinogenic amino acid side chain.

In another Formula 2 embodiment, X is —O—, n₁ is 1, 2, 3 or 4, n₂ is 1, 2 or 3 and R₅ is H. In a further embodiment, at least one occurrence of R₃ is methyl.

In one Formula 2 embodiment, X is —NH—, n₁ is 0, 1 or 2, n₂ is 1 or 2 and R₅ is H.

In another embodiment, X is —NH—, n₁ is 1, 2, 3 or 4, n₂ is 1, 2 or 3 and R₅ is H. In a further embodiment, at least one occurrence of R₃ is methyl.

In yet another Formula 2 embodiment, X is absent, n₁ is 2, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon atom.

In one Formula 2 embodiment, X is absent, n₁ is 2, and one occurrence of R₃ or R₄ is —CH₃.

In a further embodiment, R₅ is hydrogen.

In yet another Formula 2 embodiment, X is absent, n₁ is 3, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon.

In one Formula 2 embodiment, X is absent, n₁ is 2, and one occurrence of R₃ or R₄ is

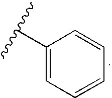

In a further embodiment, R₅ is hydrogen.

Another Formula 2 embodiment is directed to oxycodone prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₅ is hydrogen. In even a further embodiment, the proteinogenic amino acid side chain is selected from valine, leucine and isoleucine.

Yet another Formula 2 embodiment is directed to oxycodone prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₅ is hydrogen.

Itaconic acid, ketoglutaric and 2-methylene glutaric acid can also be used as a dicarboxylic acid linker in some Formula 2 embodiments. Here, R₃ and R₄ on one of the carbons defined by n₁, taken together, is a methylene group.

In one Formula 2 embodiment, the oxycodone prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another Formula 2 embodiment includes oxycodone prodrugs linked to a peptide or amino acid through a dicarboxylic acid linker substituted with an acetyl

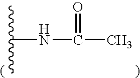

group or a carboxylic acid group. In a further embodiment, n₁ is 2 or 3 and R₅ is hydrogen. In even a further embodiment, the dicarboxylic acid linker is further substituted with an

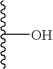

group.

In one Formula 2 embodiment, oxycodone is linked to a peptide or prodrug through a citric acid linker. The citric acid linker can be any one of 6 isomers, as provided herein in Table 2.

In another embodiment, R₅ is an opioid, and the oxycodone and the additional opioid are linked via citroyl acid linker. In this embodiment, the additional carboxylic acid in the citroyl acid linker is bound to an amino acid or peptide. In a further embodiment, the additional opioid R₅ is oxycodone.

In one embodiment, the oxycodone prodrug of the present invention uses a dicarboxylic acid disclosed in Table 1 or 2 as the dicarboxylic acid linker

In a further embodiment, the oxycodone prodrug of the present invention is selected from an oxycodone prodrug of Formulae 3, 4, 5, 6, 7, 8, 9, 10 and 11, or a pharmaceutically acceptable salt thereof. For Formulae 3-11, O₁, R₃, R₄, R₅, n₁ and n₂ are defined as provided for Formula 2.

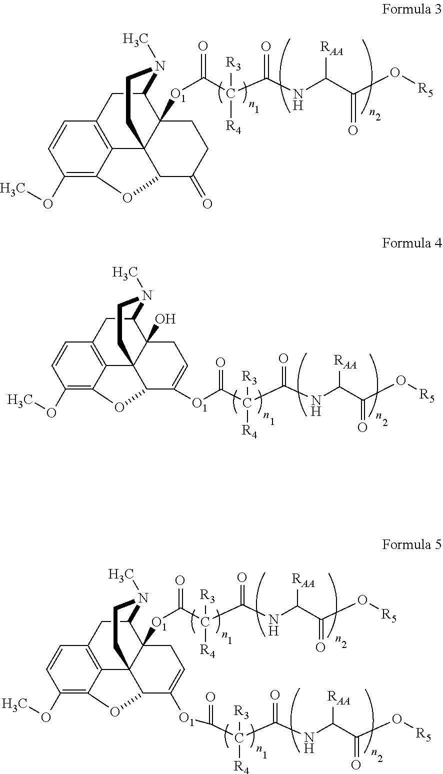

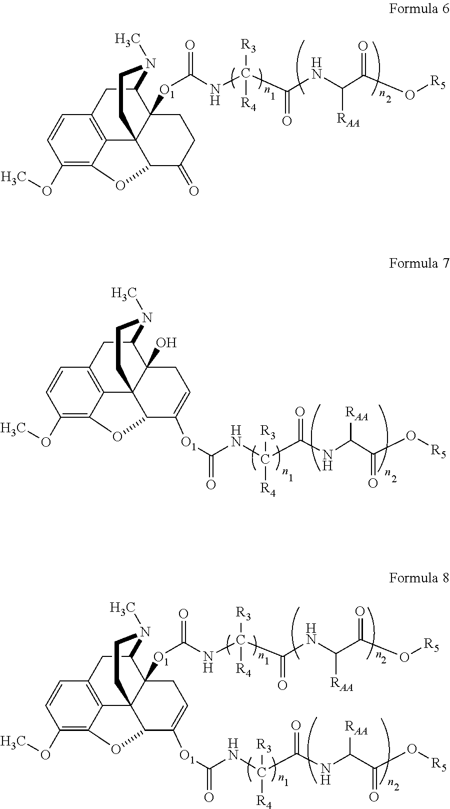

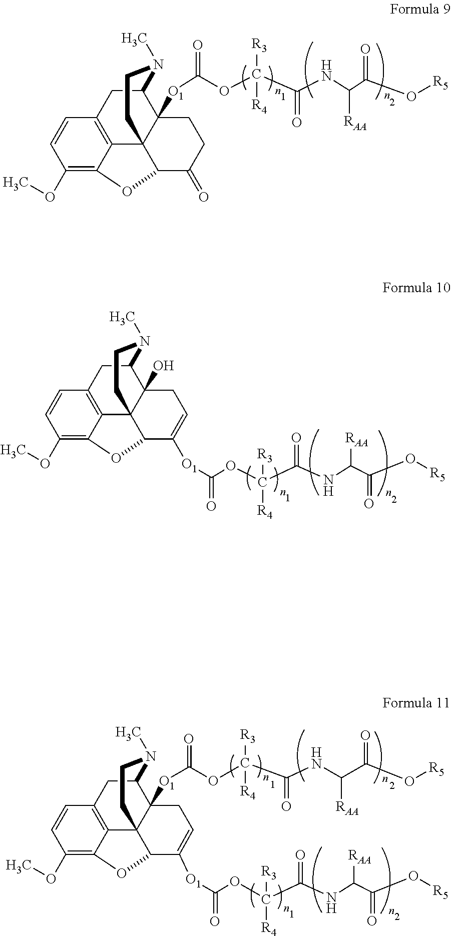

In a further Formulae 3-11 embodiment, the —N— atom in oxycodone is demethylated.

Still, in another embodiment, the oxycodone prodrug can have two prodrug moieties, where X is present in one, but absent in the other (not shown in the above formulae).

In a preferred oxycodone embodiment, the present invention is directed to oxycodone prodrugs that include a non-polar or aliphatic amino acid, including the single amino acid prodrug oxycodone-[succinyl-(S)-valine] enol ester, shown below.

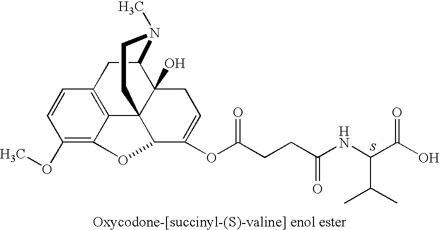

In a preferred embodiment, the single amino acid prodrug of oxycodone is the trifluoroacetate salt of oxycodone-[succinyl-(S)-valine] enol ester (Common Name (S)-2-[(3-methoxy-14-hydroxy-6,7-didehydro-4,5α-epoxy-17-methylmorphinan-6-yl) oxycarbonylpropionylamino]-3-methylbutyric acid trifluoroacetate, shown below).

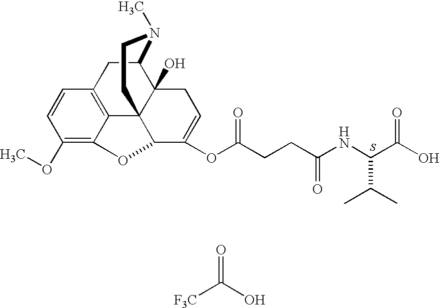

### Oxycodone-[succinyl-(S)-valine]enol ester trifluoroacetate

Other single amino acid prodrugs of oxycodone include oxycodone-[succinyl-(S)-isoleucine] enol ester, oxycodone-[succinyl-(S)-leucine]enol ester, oxycodone-[succinyl-(S)-aspartic acid] enol ester, oxycodone-[succinyl-(S)-methionine]enol ester, oxycodone-[succinyl-(S)-histidine]enol ester, oxycodone-[succinyl-(S)-tyrosine]enol ester, oxycodone-[succinyl-(S)-phenylalanine]enol ester, oxycodone-[succinyl-(S)-serine]enol ester, oxycodone-[glutaryl-(S)-valine] enol ester, oxycodone-[glutaryl-(S)-isoleucine]enol ester, and oxycodone-[glutaryl-(S)-leucine] enol ester.

In a preferred oxycodone dipeptide embodiment, the present invention is directed to the dipeptide prodrugs oxycodone-[succinyl-(S)-valine-valine]enol ester, oxycodone-[succinyl-(S)-isoleucine-isoleucine]enol ester and oxycodone-[succinyl-(S)-leucine-leucine]enol ester.

Further embodiments may include permutations drawn from these nonpolar aliphatic amino acids with the nonpolar aromatic amino acids, tryptophan and tyrosine.

Additionally, non-proteinogenic amino acid may also be used as the prodrug moiety, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above are all in the L configuration. However, the present invention also contemplates oxycodone prodrugs comprised of amino acids in the D configuration, or mixtures of amino acids in the D and L configurations.

In a preferred embodiment, the dicarboxylic acid linker is succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the opioid to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. In addition, the linkers provided in Tables 1 and 2 may be employed with oxycodone prodrugs of the present invention, for example, with the single amino acid valine.

Various oxycodone valine prodrugs are provided below, in Table 4. Valine can be readily substituted for a different amino acid, or for a peptide.

TABLE 4
Non-Limiting Examples of Oxycodone Prodrugs of the Present Invention

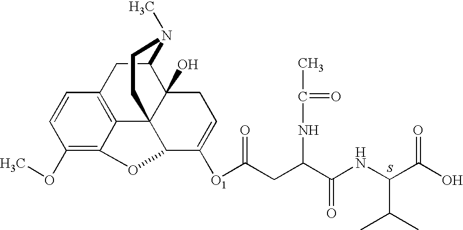

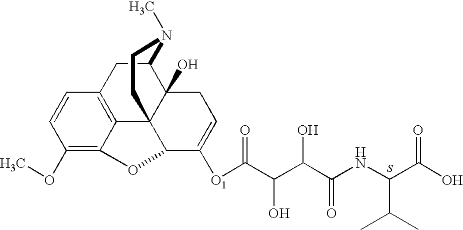

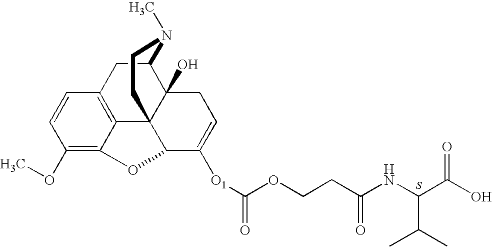

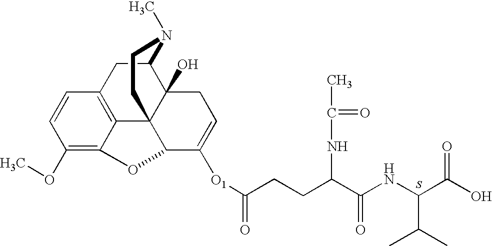

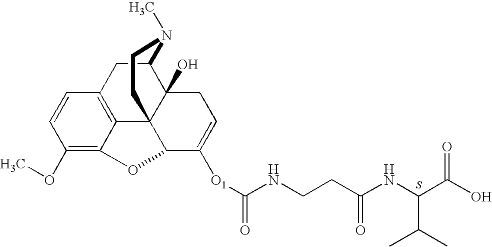

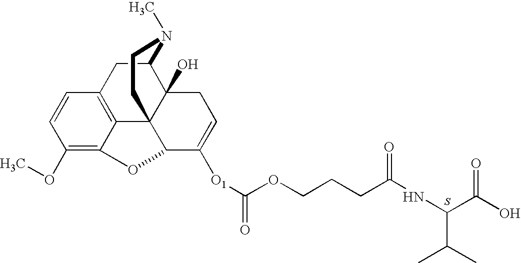

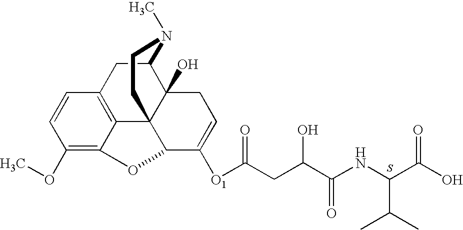

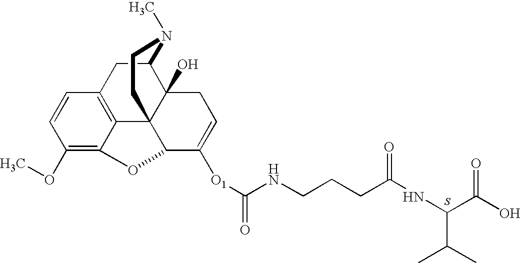

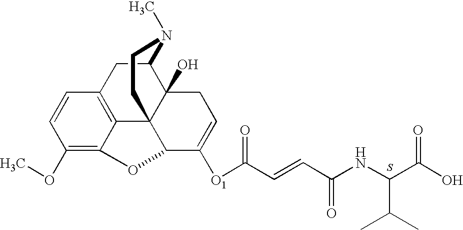

### Codeine Prodrugs of the Present Invention

In one embodiment, the prodrugs of the present invention are directed to codeine prodrugs of Formula 12, below. These codeine prodrugs are encompassed by Formula 1.

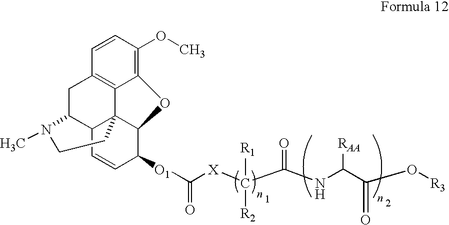

or a pharmaceutically acceptable salt thereof,

wherein,

O₁ is the hydroxyl oxygen atom present in the unbound form of codeine,

X is (—NH—), (—O—), or absent;

Each occurrence of R₁ and R₂ is independently selected from hydrogen, alkoxy,

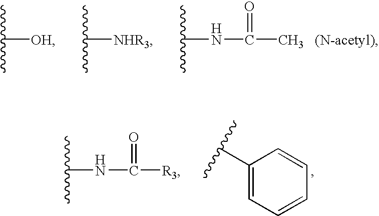

carboxyl, cycloalkyl, substituted cycloalkyl, alkyl, and substituted alkyl;

R₁ and R₂ on adjacent carbons can form a ring and R₁ and R₂ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

In the case of a double bond in the carbon chain defined by n₁, R₁ is present and R₂ is absent on the carbons that form the double bond;

R₃ is independently selected from hydrogen, alkyl, substituted alkyl, and an opioid;

When R₃ is an opioid, the —O— is a hydroxylic oxygen present in the additional opioid R₃; and

Each occurrence of R_{AA} is independently selected from a proteinogenic or non-proteinogenic amino acid side chain;

In one Formula 12 embodiment, n₁ is an integer selected from 0 to 4.

In another embodiment, X is absent and n₁ is 1, 2 or 3. In even a further embodiment, X is absent, n₁ is 1, 2 or 3, n₂ is 1 or 2 and R₁, R₂ and R₃ are each hydrogen.

In one embodiment, X is —NH—, n₁ is 0, 1, 2 or 3, n₂ is 1, 2 or 3 and R₁, R₂ and R₃ are each H. In a further embodiment, n₁ is 2.

In one embodiment, X is —O—, n₁ is 0, 1, 2 or 3, n₂ is 1, 2 or 3 and R₁, R₂ and R₃ are each H. In a further embodiment, n₁ is 2.

In one embodiment, X is absent, n₁ is 1, 2 or 3 and n₂ is 1, 2 or 3. In one embodiment, X is absent and n₁ is 1 or 2 and n₂ is 1, 2, 3, 4 or 5.

In a preferred embodiment, the prodrug moiety of a codeine compound of the present invention has one or two amino acids (i.e., n₂ is 1 or 2). In one embodiment, n₁ is 1 or 2 while n₂ is 1, 2 or 3.

In one embodiment, X is —O—, n₁ is 0, 1 or 2, n₂ is 1 or 2 and R₃ is H. In a further embodiment, at least one occurrence of R₁ is

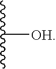

In one embodiment, X is —NH—, n₁ is 0, 1 or 2, n₂ is 1 or 2 and R₃ is H. In a further embodiment, at least one occurrence of R₁ is

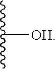

In a preferred embodiment, n₂ is 1, 2 or 3 while R₁, R₂ and R₃ are H. In another embodiment, n₂ is 1. In yet another embodiment, n₂ is 2. In yet another embodiment, n₂ is 1 or 2 and each occurrence of R_{AA} is independently a proteinogenic amino acid side chain.

In a preferred codeine embodiment, the present invention is directed to codeine prodrugs that include a non-polar or aliphatic amino acid, including the single amino acid prodrug codeine-[succinyl-(S)-valine]ester, shown below.

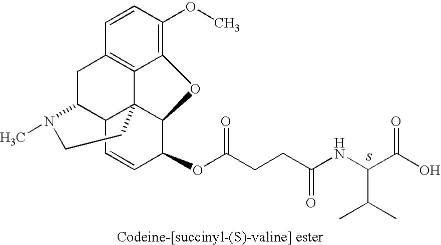

Other single amino acid prodrugs of codeine include codeine-[succinyl-(S)-isoleucine]ester, codeine-[succinyl-(S)-leucine]ester, codeine-[succinyl-(S)-aspartic acid] ester, codeine-[succinyl-(S)-methionine]ester, codeine-[succinyl-(S)-histidine]ester, codeine-[succinyl-(S)-tyrosine]ester and codeine-[succinyl-(S)-serine]ester.

In a preferred codeine dipeptide embodiment, the present invention is directed to the dipeptide prodrugs codeine-[succinyl-(S)-valine-valine]ester, codeine-[succinyl-(S)-isoleucine-isoleucine]ester and codeine-[succinyl-(S)-leucine-leucine]ester.

In another codeine embodiment, X is —O—, n₁ is 1, 2, 3 or 4, n₂ is 1, 2 or 3 and R₃ is H. In a further embodiment, at least one occurrence of R₁ is methyl.

In one codeine embodiment, X is —NH—, n₁ is 0, 1 or 2, n₂ is 1 or 2 and R₃ is H.

In another codeine embodiment, X is —NH—, n₁ is 1, 2, 3 or 4, n₂ is 1, 2 or 3 and R₃ is H. In a further embodiment, at least one occurrence of R₁ is methyl.

In yet another codeine embodiment, X is absent, n₁ is 2, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one codeine embodiment, X is absent, n₁ is 2, and one occurrence of R₁ or R₂ is —CH₃. In a further embodiment, R₃ is hydrogen.

In yet another codeine embodiment, X is absent, n₁ is 3, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one codeine embodiment, X is absent, n₁ is 2, and one occurrence of R₁ or R₂ is

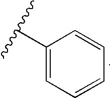

In a further embodiment, R₃ is hydrogen.

Another codeine embodiment is directed to opioid prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₃ is hydrogen. In even a further embodiment, the proteinogenic amino acid side chain is selected from valine, leucine and isoleucine.

Yet another codeine embodiment is directed to opioid prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₃ is hydrogen.

Itaconic acid, ketoglutaric and 2-methylene glutaric acid can also be used as a dicarboxylic acid linker in some codeine embodiments. Here, R₁ and R₂ on one of the carbons defined by n₁, taken together, is a methylene group.

In one codeine embodiment, the opioid prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another codeine embodiment includes opioid prodrugs linked to a peptide or amino acid through a dicarboxylic acid linker substituted with an acetyl

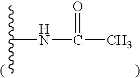

group or a carboxylic acid group. In a further embodiment, n₁ is 2 or 3 and R₃ is hydrogen. In even a further embodiment, the dicarboxylic acid linker is further substituted with an

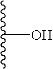

group.

In one embodiment, codeine is linked to a peptide or prodrug through a citric acid linker. The citric acid linker can be any one of 6 isomers, as provided herein in Table 2.

In one embodiment, the codeine prodrug of the present invention uses a dicarboxylic acid disclosed in Table 1 or 2 as the dicarboxylic acid linker.

In another embodiment, R₃ is an opioid, and codeine and the additional opioid are linked via citroyl acid linker. In this embodiment, the additional carboxylic acid in the citroyl acid linker is bound to an amino acid or peptide. In a further embodiment, R₃ is codeine.

In another embodiment, prodrug moiety permutations can also be drawn from valine, leucine, isoleucine, alanine and glycine. Yet further embodiments may include permutations drawn from these nonpolar aliphatic amino acids with the nonpolar aromatic amino acids, tryptophan and tyrosine.

Additionally, non-proteinogenic amino acid may also be used as the prodrug moiety in a codeine prodrug, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above for the codeine prodrug compounds are all in the L configuration. However, the present invention also contemplates codeine prodrugs comprised of amino acids in the D configuration, or mixtures of amino acids in the D and L configurations.

In a preferred codeine embodiment, the dicarboxylic acid linker is a succinyl group, derived from succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the codeine to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Other dicarboxylic acid linkers for use with codeine prodrugs of the present invention are given in Tables 1 and 2.

Various codeine-valine prodrugs are provided below, in Table 5. Valine can be readily substituted for a different amino acid, or for a peptide. In addition, as described below, the methyl group at position 3 in these molecules can be dealkylated to give a morphine prodrug.

TABLE 5
Non-Limiting Examples of Codeine Prodrugs of the Present Invention

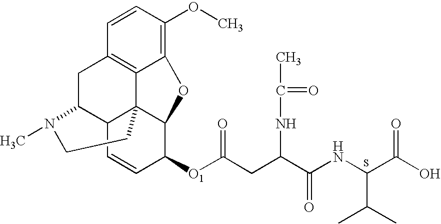

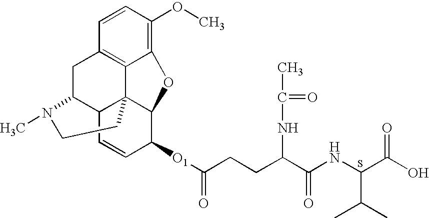

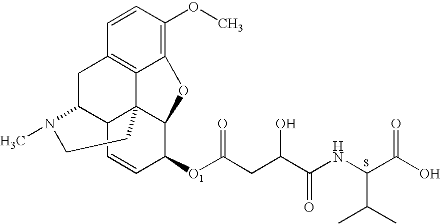

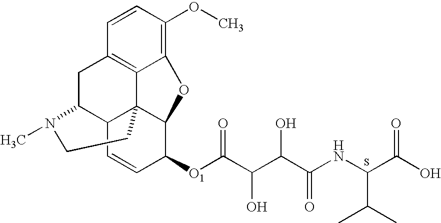

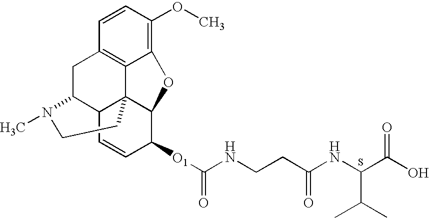

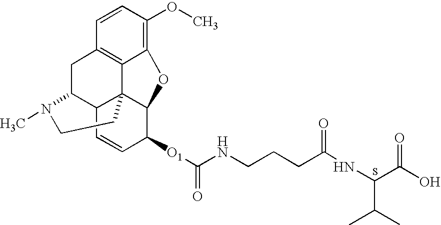

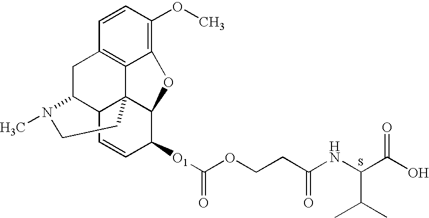

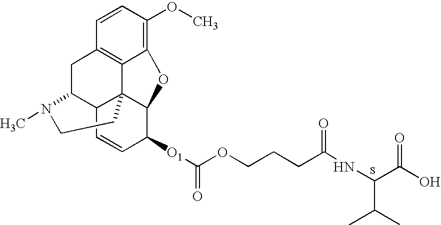

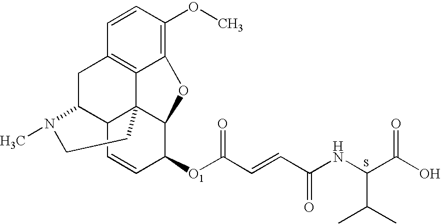

### Morphine Prodrugs of the Present Invention

The present invention also includes 3-hydroxyl derivatives of Formula 12. The 3-hydroxyl derivative of Formula 12 is a morphine prodrug. In this embodiment, morphine can have a prodrug moiety attached to either hydroxyl group, or both hydroxyl groups.

For example, single amino acid prodrugs of morphine include morphine-[succinyl-(S)-isoleucine]ester, morphine-[succinyl-(S)-leucine]ester, morphine-[succinyl-(S)-aspartic acid] ester, morphine-[succinyl-(S)-methionine]ester, morphine-[succinyl-(S)-histidine]ester, morphine-[succinyl-(S)-tyrosine]ester and morphine-[succinyl-(S)-serine]ester. The amino acid, as stated above, can be attached to the 3 position, the 6 position, or both.

In a preferred morphine dipeptide embodiment, the present invention is directed to the dipeptide pro drugs morphine-[succinyl-(S)-valine-valine]ester, morphine-[succinyl-(S)-isoleucine-isoleucine]ester and morphine-[succinyl-(S)-leucine-leucine]ester. The amino acid, as stated above, can be attached to the 3 position, the 6 position, or both.

The preferred amino acids described above for the morphine prodrug compounds are all in the L configuration. However, the present invention also contemplates morphine prodrugs comprised of amino acids in the D configuration, or mixtures of amino acids in the D and L configurations.

In a preferred morphine embodiment, the dicarboxylic acid linker is a succinyl group, derived from succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the morphine to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Other dicarboxylic acid linkers for use with morphine prodrugs of the present invention are given in Tables 1 and 2.

### Dihydrocodeine Prodrugs of the Present Invention

In one embodiment, the present invention is directed to dihydrocodeine prodrugs of Formula 13, below.

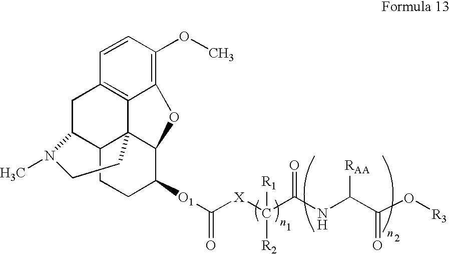

or a pharmaceutically acceptable salt thereof,

wherein,

O₁ is the phenolic oxygen atom present in the unbound dihydrocodeine,

X is (—NH—), (—O—), or absent;

Each occurrence of R₁ and R₂ is independently selected from hydrogen, alkoxy,

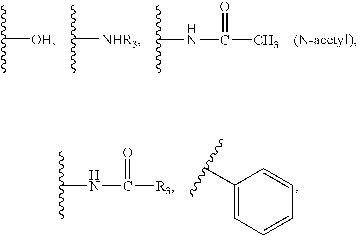

carboxyl, cycloalkyl, substituted cycloalkyl, alkyl, and substituted alkyl;

R₁ and R₂ on adjacent carbons can form a ring and R₁ and R₂ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

In the case of a double bond in the carbon chain defined by n₁, R₁ is present and R₂ is absent on the carbons that form the double bond;

R₃ is independently selected from hydrogen, alkyl, substituted alkyl and an opioid;

When R₃ is an opioid, the —O— is a hydroxylic oxygen present in the additional opioid R₃; and

Each occurrence of R_{AA} is independently selected from a proteinogenic or non-proteinogenic amino acid side chain.

In a further Formula 13 embodiment, n₁ is an integer selected from 0 to 4.

In another embodiment, X is absent and n₁ is 1, 2 or 3. In a further embodiment, X is absent, n₁ is 1, 2 or 3, n₂ is 1 or 2 and R₁, R₂ and R₃ are each hydrogen.

In one embodiment, X is —NH—, n₁ is 0, 1, 2 or 3, n₂ is 1, 2 or 3 and R₁, R₂ and R₃ are each H. In a further embodiment, n₁ is 2. In one embodiment, X is —O—, n₁ is 0, 1, 2 or 3, n₂ is 1, 2 or 3 and R₁, R₂ and R₃ are each H. In a further embodiment, n₁ is 2.

In one embodiment, X is absent, n₁ is 1, 2 or 3 and n₂ is 1, 2 or 3. In one embodiment, X is absent and n₁ is 1 or 2 and n₂ is 1, 2, 3, 4 or 5.

In a preferred embodiment, the prodrug moiety of a dihydrocodeine compound of the present invention has one or two amino acids (i.e., n₂ is 1 or 2). In one embodiment, n₁ is 1 or 2 while n₂ is 1, 2 or 3.

In one embodiment, X is —O—, n₁ is 0, 1 or 2, n₂ is 1 or 2 and R₃ is H. In a further embodiment, at least one occurrence of R₁ is

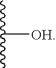

In one embodiment, X is —NH—, n₁ is 0, 1 or 2, n₂ is 1 or 2 and R₃ is H. In a further embodiment, at least one occurrence of R₁ is

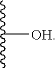

In a preferred embodiment, n₂ is 1, 2 or 3 while R₁, R₂ and R₃ are H. In another embodiment, n₂ is 1. In yet another embodiment, n₂ is 2. In yet another embodiment, n₂ is 1 or 2 and each occurrence of R_{AA} is independently a proteinogenic amino acid side chain.

In a preferred dihydrocodeine embodiment, the present invention is directed to dihydrocodeine prodrugs that include a non-polar or aliphatic amino acid, including the single amino acid prodrug dihydrocodeine-[succinyl-(S)-valine]ester, shown below.

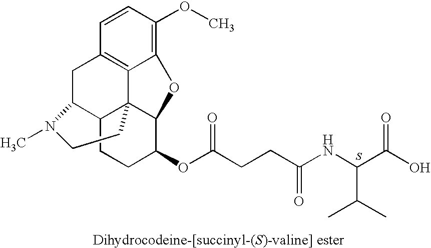

Other single amino acid prodrugs of dihydrocodeine include dihydrocodeine-[succinyl-(S)-isoleucine]ester, dihydrocodeine-[succinyl-(S)-leucine]ester, dihydrocodeine-[succinyl-(S)-aspartic acid] ester, dihydrocodeine-[succinyl-(S)-methionine]ester, dihydrocodeine-[succinyl-(S)-histidine]ester, dihydrocodeine-[succinyl-(S)-tyrosine]ester and dihydrocodeine-[succinyl-(S)-serine]ester.

In a preferred dihydrocodeine dipeptide embodiment, the present invention is directed to the dipeptide prodrugs dihydrocodeine-[succinyl-(S)-valine-valine]ester, dihydrocodeine-[succinyl-(S)-isoleucine-isoleucine]ester and dihydrocodeine-[succinyl-(S)-leucine-leucine]ester.

In another dihydrocodeine embodiment, X is —O—, n₁ is 1, 2, 3 or 4, n₂ is 1, 2 or 3 and R₃ is H. In a further embodiment, at least one occurrence of R₁ is methyl.

In one dihydrocodeine embodiment, X is —NH—, n₁ is 0, 1 or 2, n₂ is 1 or 2 and R₃ is H.

In another dihydrocodeine embodiment, X is —NH—, n₁ is 1, 2, 3 or 4, n₂ is 1, 2 or 3 and R₃ is H. In a further embodiment, at least one occurrence of R₁ is methyl.

In yet another dihydrocodeine embodiment, X is absent, n₁ is 2, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one dihydrocodeine embodiment, X is absent, n₁ is 2, and one occurrence of R₁ or R₂ is —CH₃. In a further embodiment, R₃ is hydrogen.

In yet another dihydrocodeine embodiment, X is absent, n₁ is 3, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one dihydrocodeine embodiment, X is absent, n₁ is 2, and one occurrence of R₁ or R₂ is

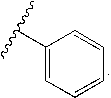

In a further embodiment, R₃ is hydrogen.

Another dihydrocodeine embodiment is directed to opioid prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₃ is hydrogen. In even a further embodiment, the proteinogenic amino acid side chain is selected from valine, leucine and isoleucine.

Yet another dihydrocodeine embodiment is directed to opioid prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₃ is hydrogen.

Itaconic acid, ketoglutaric and 2-methylene glutaric acid can also be used as a dicarboxylic acid linker in some dihydrocodeine embodiments. Here, R₁ and R₂ on one of the carbons defined by n₁, taken together, is a methylene group.

In one dihydrocodeine embodiment, the opioid prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another dihydrocodeine embodiment includes opioid prodrugs linked to a peptide or amino acid through a dicarboxylic acid linker substituted with an acetyl

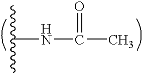

group or a carboxylic acid group. In a further embodiment, n₁ is 2 or 3 and R₃ is hydrogen. In even a further embodiment, the dicarboxylic acid linker is further substituted with an

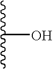

group.

In one embodiment, dihydrocodeine is linked to a peptide or prodrug through a citric acid linker. The citric acid linker can be any one of 6 isomers, as provided herein in Table 2.

In one embodiment, the dihydrocodeine prodrug of the present invention uses a dicarboxylic acid disclosed in Table 1 or 2 as the dicarboxylic acid linker.

In another embodiment, R₃ is an opioid, and dihydrocodeine and the additional opioid are linked via citroyl acid linker. In this embodiment, the additional carboxylic acid in the citroyl acid linker is bound to an amino acid or peptide. In a further embodiment, R₃ is dihydrocodeine.

In another embodiment, dihydrocodeine prodrug moiety permutations can be drawn from valine, leucine, isoleucine, alanine and glycine. Yet further embodiments may include permutations drawn from these nonpolar aliphatic amino acids with the nonpolar aromatic amino acids, tryptophan and tyrosine.

Additionally, non-proteinogenic amino acid may also be used as the prodrug moiety in a dihydrocodeine prodrug, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above for the dihydrocodeine prodrug compounds are all in the L configuration. However, the present invention also contemplates prodrugs of Formula 13 comprised of amino acids in the D configuration, or mixtures of amino acids in the D and L configurations.

In a preferred dihydrocodeine embodiment, the dicarboxylic acid linker is a succinyl group, derived from succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the dihydrocodeine to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Other dicarboxylic acid linkers for use with dihycdrocodeine prodrugs of the present invention are given in Tables 1 and 2.

Various dihydrocodeine-valine prodrugs are provided below, in Table 6. Valine can be readily substituted for a different amino acid, or for a peptide.

TABLE 6
Non-Limiting Examples of Dihydrocodeine
Prodrugs of the Present Invention

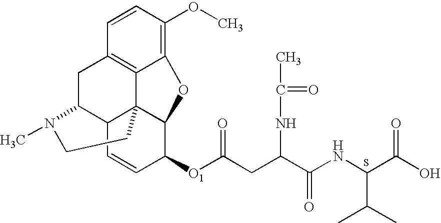

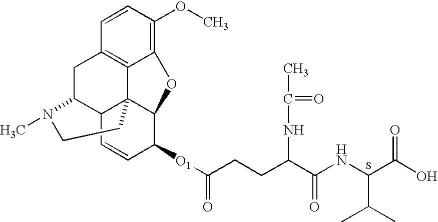

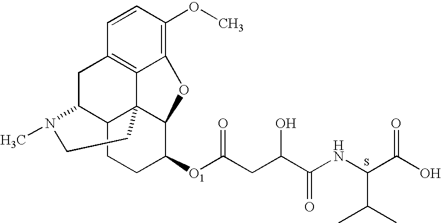

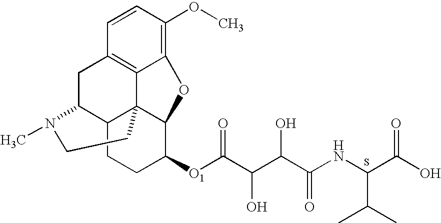

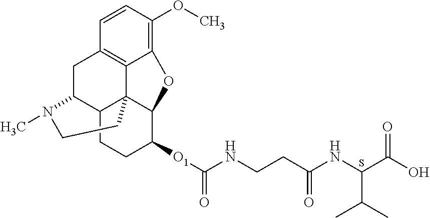

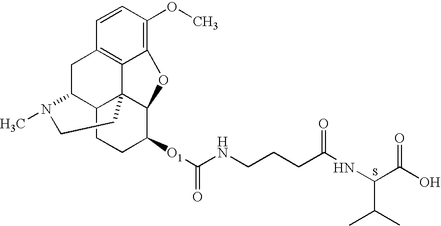

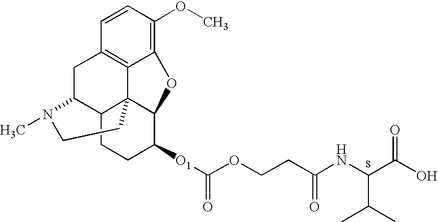

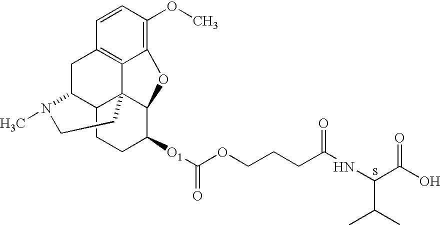

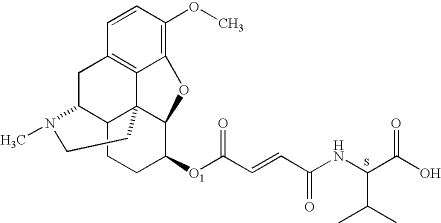

The present invention also includes 3-hydroxyl (OH) derivatives of each of the aforementioned dihydrocodeine prodrugs (i.e., where the 3-methoxy group is replaced with a 3-hydroxy group). 3-OH dihydrocodeine is known to be an active metabolite of dihydrocodeine.

Therefore, in one embodiment, the present invention is directed to a demethylated prodrug of Formula 13, wherein the demethylation occurs at position 3. The present invention encompasses the Formula 13 embodiments described above, wherein position 3 has been demethylated, and replaced with an —OH group. Alternatively or additionally, the nitrogen atom can be demethylated.

In another embodiment, a demethylated dihydrocodeine metabolite prodrug is provided, wherein the 3-OH group is attached to a peptide or amino acid via a dicarboxylic acid linker. Various dicarboxylic acid linkers for use with a dihydrocodeine metabolite prodrug are given it Tables 1 and 2.

In yet another embodiment, a dipeptide prodrug is provided, wherein a prodrug moiety is present both at the 6 position and at the 3 position of dihydrocodeine.

Hydromorphone Prodrugs of the Present Invention

Hydromorphone prodrugs of the present invention are encompassed by Formula 14, below.

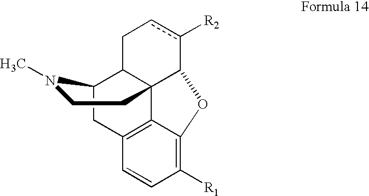

or a pharmaceutically acceptable salt thereof,

wherein,

R₁ is independently selected from

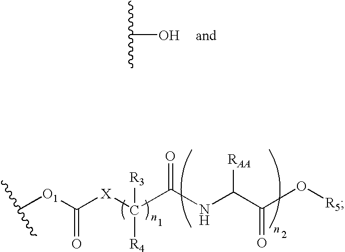

R₂ is selected from

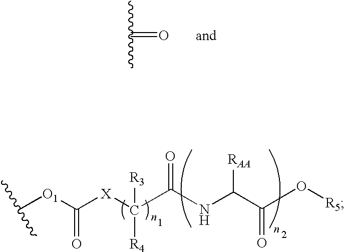

Each occurrence of O₁ is independently an oxygen atom present in the unbound form of hydromorphone;

Each occurrence of X is independently (—NH—), (—O—), or absent;

Each occurrence of R₃ and R₄ is independently selected from hydrogen, alkoxy,

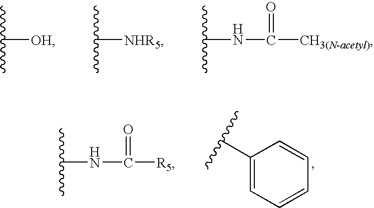

carboxyl, cycloalkyl, substituted cycloalkyl, alkyl, substituted alkyl;

R₃ and R₄ on adjacent carbons can form a ring and R₃ and R₄ on the same carbon, taken together, can be a methylene group;

Each occurrence of n₁ is independently an integer selected from 0 to 16 and each occurrence of n₂ is independently an integer selected from 1 to 9, and each occurrence of n₁ and n₂ can be the same or different;

the carbon chain defined by n₁ can include a cycloalkyl or aromatic ring;

In the case of a double bond in the carbon chain defined by n₁, R₃ is present and R₄ is absent on the carbons that form the double bond;

Each occurrence of R₅ is independently selected from hydrogen, alkyl, substituted alkyl, and an opioid;

When R₅ is an opioid, the —O— is a hydroxylic oxygen present in the additional opioid R₅;

Each occurrence of R_{AA} is independently selected from a proteinogenic or non-proteinogenic amino acid side chain;

the dashed line in Formula 14 is absent when R₂ is

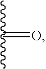

and a bond when R₂ is not

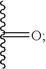

and

at least one of R₁ and R₂ is

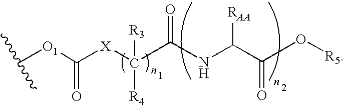

In a further Formula 14 embodiment, n₁ is an integer selected from 0 to 4.

In another Formula 14 embodiment, R₂ is

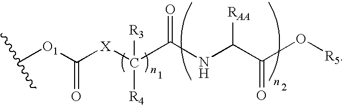

In a further embodiment, X is absent and n₁ is 1, 2 or 3.

In one embodiment, R₁ is

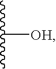

X is absent, n₁ is 0, 1, 2 or 3, n₂ is 1, 2 or 3 and R₃, R₄ and R₅ are each H. In a further embodiment, n₁ is 2. In one embodiment, R₂ is

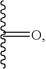

X is absent, n₁ is 0, 1, 2 or 3, n₂ is 1, 2 or 3 and R₃, R₄ and R₅ are each H. In a further embodiment, n₁ is 2.

In one embodiment, R₁ is

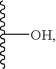

X is absent, n₁ is 0, 1, 2, 3 or 4, n₂ is 1, 2, 3, 4 or 5 and R₃, R₄ and R₅ are each H. In a further embodiment, n₁ is 2. In one embodiment, R₂ is

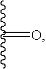

X is absent, n₁ is 0, 1, 2, 3 or 4, n₂ is 1, 2, 3, 4 or 5 and R₃, R₄ and R₅ are each H. In a further embodiment, n₁ is 2.

In one embodiment, X is —O—, n₁ is 0, 1 or 2, n₂ is 1 or 2 and R₅ is H. In a further embodiment, n₁ is 2 and R₁ is

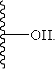

In one embodiment, X is —NH—, n₁ is 0, 1 or 2, n₂ is 1 or 2 and R₅ is H. In a further embodiment, n₁ is 2 and R₁ is

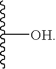

In one embodiment, the hydromorphone prodrug of the present invention has two prodrug moieties and each occurrence of n₁ is selected from 0, 1, 2, 3 or 4. In a further embodiment, at least one occurrence of n₂ is 1, 2 or 3.

In one embodiment, at least one occurrence of n₁ is 1 or 2 and at least one occurrence of n₂ is 1, 2, 3, 4 or 5. In a further embodiment, there is only one occurrence of n₁ and one occurrence of n₂.

In a preferred embodiment, the hydromorphone compound of the present invention has a single prodrug moiety, and the prodrug moiety has one or two amino acids (i.e., n₂ is 1 or 2). In a further embodiment, R₁ is

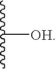

Alternatively, in another embodiment, R₂ is

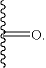

In one embodiment, the hydromorphone compound has one prodrug moiety and, X is —O—, n₁ is 0, 1 or 2, n₂ is 1 or 2 and R₅ is H. In another hydromorphone embodiment, the compound has one prodrug moiety, X is —NH—, n₁ is 0, 1 or 2, n₂ is 1 or 2 and R₅ is H.

In one embodiment, the hydromorphone compound of the present invention has a single prodrug moiety, and n₁ is 1 or 2 while n₂ is 1, 2 or 3.

In a preferred embodiment, n₂ is 1, 2 or 3 while R₃, R₄ and R₅ are H. In another embodiment, n₂ is 1. In yet another embodiment, n₂ is 2. In yet another embodiment, n₂ is 1 or 2 and each occurrence of R_{AA} is independently a proteinogenic amino acid side chain.

In another hydromorphone embodiment, X is —O—, n₁ is 1, 2, 3 or 4, n₂ is 1, 2 or 3 and R₅ is H. In a further embodiment, at least one occurrence of R₃ is methyl.

In one hydromorphone embodiment, X is —NH—, n₁ is 0, 1 or 2, n₂ is 1 or 2 and R₅ is H.

In another hydromorphone embodiment, X is —NH—, n₁ is 1, 2, 3 or 4, n₂ is 1, 2 or 3 and R₅ is H. In a further embodiment, at least one occurrence of R₃ is methyl.

In yet another hydromorphone embodiment, X is absent, n₁ is 2, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon atom.

In one hydromorphone embodiment, X is absent, n₁ is 2, and one occurrence of R₃ or R₄ is —CH₃. In a further embodiment, R₅ is hydrogen.

In yet another hydromorphone embodiment, X is absent, n₁ is 3, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon.

In one hydromorphone embodiment, X is absent, n₁ is 2, and one occurrence of R₃ or R₄ is

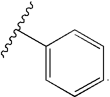

In a further embodiment, R₅ is hydrogen.

Another hydromorphone embodiment is directed to hydromorphone prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₅ is hydrogen. In even a further embodiment, the proteinogenic amino acid side chain is selected from valine, leucine and isoleucine.

Yet another hydromorphone embodiment is directed to hydromorphone prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₅ is hydrogen.

Itaconic acid, ketoglutaric and 2-methylene glutaric acid can also be used as a dicarboxylic acid linker in some hydromorphone embodiments. Here, R₃ and R₄ on one of the carbons defined by n₁, taken together, is a methylene group.

In one hydromorphone embodiment, the hydromorphone prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another hydromorphone embodiment includes hydromorphone prodrugs linked to a peptide or amino acid through a dicarboxylic acid linker substituted with an acetyl

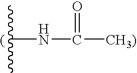

group or a carboxylic acid group. In a further hydromorphone embodiment, n₁ is 2 or 3 and R₃ is hydrogen. In even a further embodiment, the dicarboxylic acid linker is further substituted with an

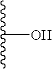

group.

In one embodiment, hydromorphone is linked to a peptide or prodrug through a citric acid linker. The citric acid linker can be any one of 6 isomers, as provided herein in Table 2.

In one embodiment, the hydromorphone prodrug of the present invention uses a dicarboxylic acid disclosed in Table 1 or 2 as the dicarboxylic acid linker.

In another embodiment, R₅ is an opioid, and hydromorphone and the additional opioid are linked via citroyl acid linker. In this embodiment, the additional carboxylic acid in the citroyl acid linker is bound to an amino acid or peptide. In a further embodiment, the additional opioid R₅ is hydromorphone.

In a further embodiment, the hydromorphone prodrug of the present invention is selected from an hydromorphone prodrug of Formula 15, 16, 17, 18, 19, 20, 21, 22 and 23, or a pharmaceutically acceptable salt thereof. For Formulae 15-23 O₁, R₃, R₄, R₅, n₁ and n₂ are defined as given for Formula 14.

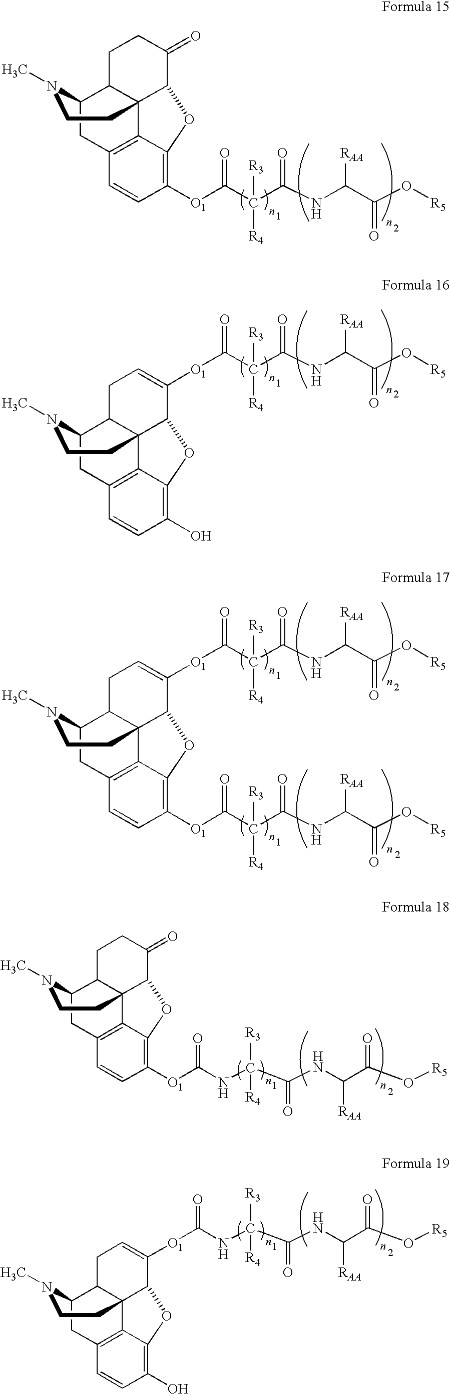

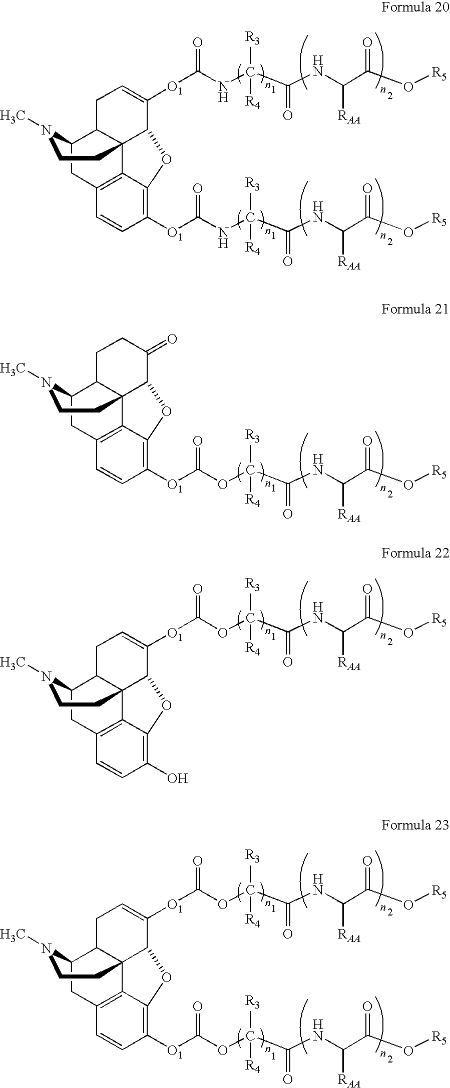

In a further Formulae 15-23 embodiment, the —N— atom in hydromorphone is demethylated.

Preferred embodiments of the hydromorphone prodrugs of the present invention are prodrugs wherein the side chain comprises a non-polar or an aliphatic amino acid, including the single amino acid prodrug hydromorphone-[succinyl-(S)-valine]ester, shown below.

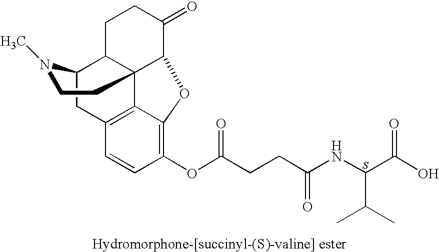

Other single amino acid prodrugs of hydromorphone include hydromorphone-[succinyl-(S)-isoleucine]ester, hydromorphone-[succinyl-(S)-leucine]ester, hydromorphone-[succinyl-(S)-aspartic acid] ester, hydromorphone-[succinyl-(S)-methionine]ester, hydromorphone-[succinyl-(S)-histidine]ester, hydromorphone-[succinyl-(S)-tyrosine]ester and hydromorphone-[succinyl-(S)-serine]ester.

In a preferred hydromorphone dipeptide embodiment, the present invention is directed to the dipeptide prodrugs hydromorphone-[succinyl-(S)-valine-valine]ester, hydromorphone-[succinyl-(S)-isoleucine-isoleucine]ester and hydromorphone-[succinyl-(S)-leucine-leucine]ester.

In another embodiment, hydromorphone prodrug moiety permutations can be drawn from valine, leucine, isoleucine, alanine and glycine. Yet further embodiments may include permutations drawn from these nonpolar aliphatic amino acids with the nonpolar aromatic amino acids, tryptophan and tyrosine.

Additionally, non-proteinogenic amino acid may also be used as the prodrug moiety in a hydromorphone prodrug, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above for the hydromorphone prodrug compounds are all in the L configuration. However, the present invention also contemplates prodrugs of Formulae 14-23 comprised of amino acids in the D configuration, or mixtures of amino acids in the D and L configurations.

In a preferred hydromorphone embodiment, the dicarboxylic acid linker is a succinyl group, derived from succinic acid. Other dicarboxylic acid linkers within the scope of the invention include, but are not limited to, malonic acid, glutaric acid, adipic acid, or other longer chain dicarboxylic acids or substituted derivatives thereof (for example, see Table 1).

As alternatives to the use of a dicarboxylic acid linker to attach the hydromorphone to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Other examples of suitable linkers for use with hydromorphone prodrugs of the present invention are given in Tables 1 and 2.

Buprenorphine Prodrugs of the Present Invention

In one embodiment, prodrugs of the present invention are directed to novel buprenorphine prodrugs of Formula 24, below.

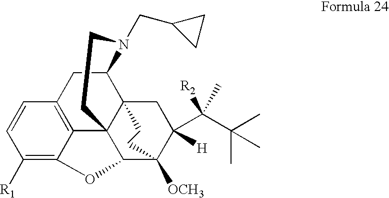

or a pharmaceutically acceptable salt thereof,

wherein,

R₁ and R₂ are independently selected from

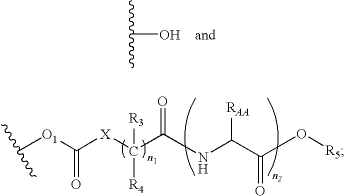

Each occurrence of O₁ is independently an oxygen atom present in the unbound form of buprenorphine;

Each occurrence of X is independently (—NH—), (—O—), or absent;

Each occurrence of R₃ and R₄ is independently selected from hydrogen, alkoxy,

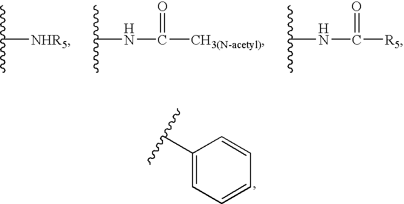

carboxyl, cycloalkyl, substituted cycloalkyl, alkyl, and substituted alkyl;

R₃ and R₄ on adjacent carbons can form a ring and R₃ and R₄ on the same carbon, taken together, can be a methylene group;

Each occurrence of n₁ is independently an integer selected from 0 to 16 and each occurrence of n₂ is independently an integer selected from 1 to 9;

the carbon chain defined by n₁ can include a cycloalkyl or aromatic ring;

In the case of a double bond in the carbon chain defined by n₁, R₃ is present and R₄ is absent on the carbons that form the double bond;

Each occurrence of R₅ is independently selected from hydrogen, alkyl, substituted alkyl, and an opioid;

When R₅ is an opioid, the —O— is a hydroxylic oxygen present in the additional opioid R₅;

Each occurrence of R_{AA} is independently selected from a proteinogenic or non-proteinogenic amino acid side chain; and

at least one of R₁ and R₂ is

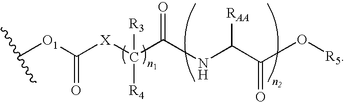

In a further Formula 24 embodiment, at least one occurrence of n₁ is an integer selected from 0 to 4. In yet a further embodiment, the prodrug is N-dealkylated (i.e., a norbuprenorphine prodrug).

In another Formula 24 embodiment, R₂ is

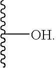

In a further embodiment, X is absent and n₁ is 1, 2 or 3. In yet a further embodiment, the prodrug is N-dealkylated (i.e., a norbuprenorphine prodrug).

In one embodiment, R₁ is

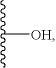

X is absent, n₁ is 0, 1, 2 or 3, n₂ is 1, 2 or 3 and R₃, R₄ and R₅ are each H. In a further embodiment, n₁ is 2. In yet a further embodiment, the prodrug is N-dealkylated (i.e., a norbuprenorphine prodrug).

In one embodiment, R₁ is

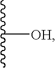

X is absent, n₁ is 0, 1, 2 or 3, n₂ is 1, 2, 3, 4 or 5 and R₃, R₄ and R₅ are each H. In a further embodiment, n₁ is 2. In yet a further embodiment, the prodrug is N-dealkylated (i.e., a norbuprenorphine prodrug).

In one embodiment, R₂ is

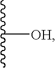

X is absent, n₁ is 0, 1, 2 or 3, n₂ is 1, 2 or 3 and R₃, R₄ and R₅ are each H. In a further embodiment, n₁ is 2. In one embodiment, R₂ is

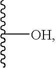

X is absent, n₁ is 0, 1, 2 or 3, n₂ is 1, 2, 3, 4 or 5 and R₃, R₄ and R₅ are each H. In a further embodiment, n₁ is 2. In yet a further embodiment, the prodrug is N-dealkylated (i.e., a norbuprenorphine prodrug).

In one embodiment, R₂ is

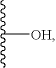

X is —O—, n₁ is 0, 1 or 2, n₂ is 1 or 2 and R₅ is H. In a further embodiment, n₁ is 2. In one embodiment, R₂ is

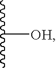

X is —NH—, n₁ is 0, 1 or 2, n₂ is 1 or 2 and R₅ is H. In a further embodiment, n₁ is 2. In yet a further embodiment, the prodrug is N-dealkylated (i.e., a norbuprenorphine prodrug).

In one embodiment, X is absent and n₁ is 1, 2 or 3 and n₂ is 1, 2 or 3. In a further embodiment, R₂ is

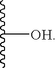

In another embodiment, X is absent n₁ is 1 or 2 and n₂ is 1, 2, 3, 4 or 5. In a further embodiment, R₂ is

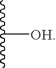

In a further embodiment, the prodrug is N-dealkylated (i.e., a norbuprenorphine prodrug).

In one embodiment, R₂ is

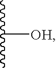

n₁ is 1, 2 or 3, n₂ is 1 or 2 and at least one occurrence of R₃ is

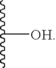

In a further embodiment, the prodrug is N-dealkylated (i.e., a norbuprenorphine prodrug).

In a preferred embodiment, the buprenorphine prodrug of the present invention has one prodrug moiety, and the prodrug moiety has one or two amino acids (i.e., n₂ is 1 or 2). In one embodiment, the buprenorphine prodrug of the present invention has one prodrug moiety, and n₁ is 1 or 2 while n₂ is 1, 2 or 3. In a further embodiment, the prodrug is N-dealkylated (i.e., a norbuprenorphine prodrug).

In a preferred embodiment, n₂ is 1, 2 or 3 while R₃, R₄ and R₅ are H. In another embodiment, n₂ is 1. In yet another embodiment, n₂ is 2. In yet another embodiment, n₂ is 1 or 2 and each occurrence of R_{AA} is independently a proteinogenic amino acid side chain. In a further embodiment, the prodrug is N-dealkylated (i.e., a norbuprenorphine prodrug).

In another buprenorphine embodiment, X is —O—, n₁ is 1, 2, 3 or 4, n₂ is 1, 2 or 3 and R₅ is H. In a further embodiment, at least one occurrence of R₃ is methyl.

In one buprenorphine embodiment, X is —NH—, n₁ is 0, 1 or 2, n₂ is 1 or 2 and R₅ is H.

In another buprenorphine embodiment, X is —NH—, n₁ is 1, 2, 3 or 4, n₂ is 1, 2 or 3 and R₅ is H. In a further embodiment, at least one occurrence of R₃ is methyl.

In yet another buprenorphine embodiment, X is absent, n₁ is 2, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon atom.

In one buprenorphine embodiment, X is absent, n₁ is 2, and one occurrence of R₃ or R₄ is —CH₃. In a further embodiment, R₅ is hydrogen.

In yet another buprenorphine embodiment, X is absent, n₁ is 3, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon.

In one buprenorphine embodiment, X is absent, n₁ is 2, and one occurrence of R₃ or R₄ is

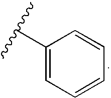

In a further embodiment, R₅ is hydrogen.

Another buprenorphine embodiment is directed to buprenorphine prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₅ is hydrogen. In even a further embodiment, the proteinogenic amino acid side chain is selected from valine, leucine and isoleucine.

Yet another buprenorphine embodiment is directed to buprenorphine prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₅ is hydrogen.

Itaconic acid, ketoglutaric and 2-methylene glutaric acid can also be used as a dicarboxylic acid linker in some buprenorphine embodiments. Here, R₃ and R₄ on one of the carbons defined by n₁, taken together, is a methylene group.

In one buprenorphine embodiment, the buprenorphine prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another buprenorphine embodiment includes buprenorphine prodrugs linked to a peptide or amino acid through a dicarboxylic acid linker substituted with an acetyl

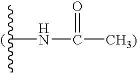

group or a carboxylic acid group. In a further buprenorphine embodiment, n₁ is 2 or 3 and R₃ is hydrogen. In even a further embodiment, the dicarboxylic acid linker is further substituted with an

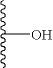

group.

In one embodiment, buprenorphine is linked to a peptide or prodrug through a citric acid linker. The citric acid linker can be any one of 6 isomers, as provided herein in Table 2.

In one embodiment, the buprenorphine prodrug of the present invention uses a dicarboxylic acid disclosed in Table 1 or 2 as the dicarboxylic acid linker

In another embodiment, R₅ is an opioid, and the buprenorphine and the additional opioid are linked via citroyl acid linker. In this embodiment, the additional carboxylic acid in the citroyl acid linker is bound to an amino acid or peptide. In a further embodiment, the additional opioid R₅ is buprenorphine.

In another embodiment, the buprenorphine prodrug of the present invention is selected from an buprenorphine prodrug of Formulae 25, 26, 27, 28, 29, 30, 31, 32 and 33, a pharmaceutically acceptable salt thereof, or an N-dealkylated derivative thereof (i.e., a norbuprenorphine prodrug). For Formulae 25-33, O₁, R₃, R₄, R₅, n₁ and n₂ are defined as given for Formula 24.

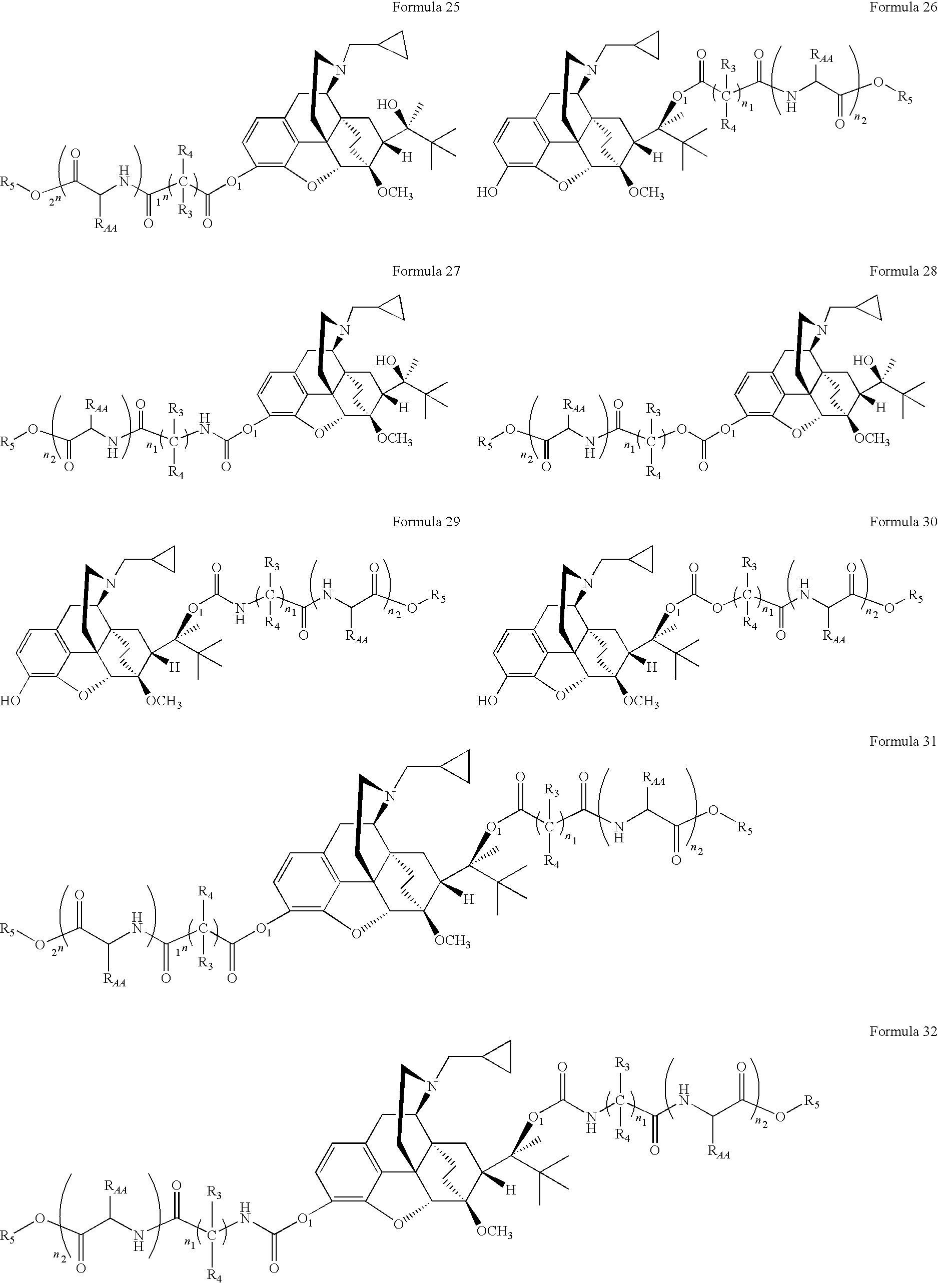

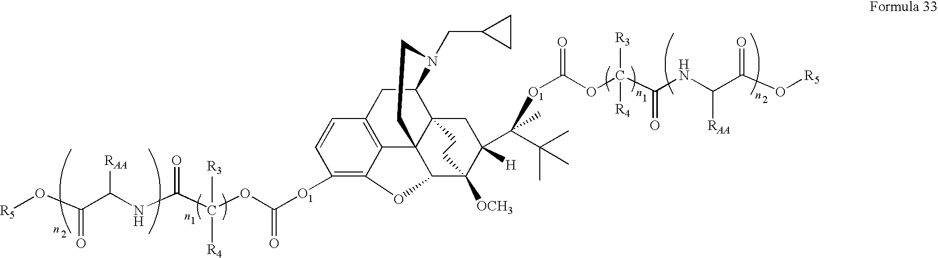

Still, in another embodiment, the buprenorphine prodrug can have two prodrug moieties, wherein X is present in one, but absent in the other (not shown in the above formulae). In a further embodiment, the buprenorphine dipeptide prodrug is N-dealkylated, i.e., the dipepetide prodrug is a norbuprenorphine prodrug.

Preferred embodiments of the buprenorphine prodrugs of the present invention are prodrugs wherein the side chain comprises a non-polar or an aliphatic amino acid, including the single amino acid prodrugs buprenorphine succinyl valine ester, and norbuprenorphine succinyl valine ester, shown below.

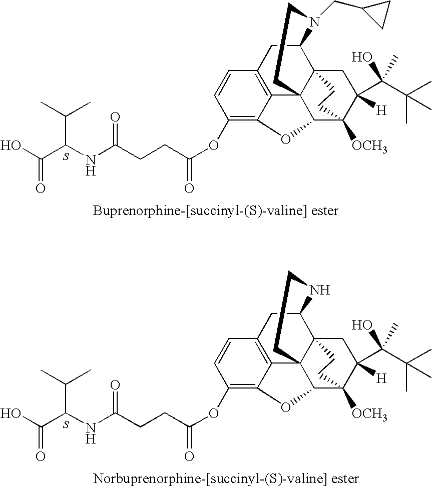

Other single amino acid prodrugs of buprenorphine include buprenorphine-[succinyl-(S)-isoleucine]ester, buprenorphine-[succinyl-(S)-leucine]ester, buprenorphine-[succinyl-(S)-aspartic acid] ester, buprenorphine-[succinyl-(S)-methionine]ester, buprenorphine-[succinyl-(S)-histidine]ester, buprenorphine-[succinyl-(S)-tyrosine]ester and buprenorphine-[succinyl-(S)-serine]ester.

Other single amino acid prodrugs of norbuprenorphine include norbuprenorphine-[succinyl-(S)-isoleucine]ester, norbuprenorphine-[succinyl-(S)-leucine]ester, norbuprenorphine-[succinyl-(S)-aspartic acid] ester, norbuprenorphine-[succinyl-(S)-methionine]ester, norbuprenorphine-[succinyl-(S)-histidine]ester, norbuprenorphine-[succinyl-(S)-tyrosine]ester and norbuprenorphine-[succinyl-(S)-serine]ester.

In a preferred buprenorphine dipeptide embodiment, the present invention is directed to the dipeptide pro drugs buprenorphine-[succinyl-(S)-valine-valine]ester, buprenorphine-[succinyl-(S)-isoleucine-isoleucine]ester and buprenorphine-[succinyl-(S)-leucine-leucine]ester.

In another embodiment, buprenorphine and norbuprenorphine prodrug moiety permutations can be drawn from valine, leucine, isoleucine, alanine and glycine. Yet further embodiments may include permutations drawn from these nonpolar aliphatic amino acids with the nonpolar aromatic amino acids, tryptophan and tyrosine.

Additionally, non-proteinogenic amino acid may also be used as the prodrug moiety in a buprenorphine or norbuprenorphine prodrug, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above for the buprenorphine prodrug compounds (and norbuprenorphine) are all in the L configuration. However, the present invention also contemplates buprenorphine prodrugs comprised of amino acids in the D configuration, or mixtures of amino acids in the D and L configurations.

In a preferred buprenorphine embodiment, the dicarboxylic acid linker is derived from succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the buprenorphine or norbuprenorphine to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Examples of dicarboxylic acid linkers for use with the buprenorphine prodrugs of the present invention are given in Tables 1 and 2. These can be conjugated to an amino acid or short peptide, for example, valine.

### Oxymorphone Prodrugs of the Present Invention

In one embodiment, prodrugs of the present invention are directed to novel oxymorphone prodrugs of Formula 34, below.

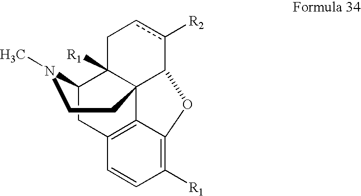

or a pharmaceutically acceptable salt thereof,

wherein,

Each occurrence of R₁ is independently selected from

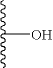

and, and each occurrence of R₁ can be the same or different;

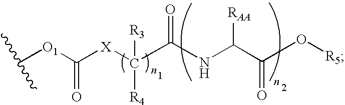

R₂ is selected from

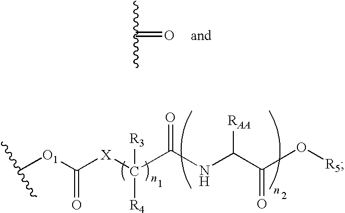

Each occurrence of O₁ is independently an oxygen atom present in the unbound form of oxymorphone;

Each occurrence of X is independently (—NH—), (—O—), or absent;

Each occurrence of R₃ and R₄ is independently selected from hydrogen, alkoxy,

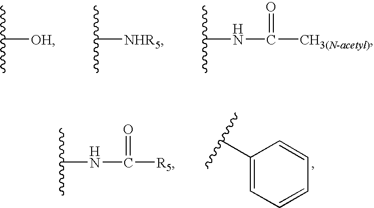

carboxyl, cycloalkyl, substituted cycloalkyl, alkyl, and substituted alkyl;

R₃ and R₄ on adjacent carbons can form a ring and R₃ and R₄ on the same carbon, taken together, can be a methylene group;

Each occurrence of n₁ is independently an integer selected from 0 to 16 and each occurrence of n₂ is independently an integer selected from 1 to 9;

the carbon chain defined by n₁ can include a cycloalkyl or aromatic ring;

In the case of a double bond in the carbon chain defined by n₁, R₃ is present and R₄ is absent on the carbons that form the double bond;

Each occurrence of R₅ is independently selected from hydrogen, alkyl, substituted alkyl, and an opioid;

When R₅ is an opioid, the —O— is a hydroxylic oxygen present in the additional opioid R₅;

Each occurrence of R_{AA} is independently selected from a proteinogenic or non-proteinogenic amino acid side chain;

the dashed line in Formula 34 is absent when R₂ is

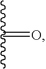

and a bond when R₂ is not

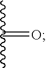

and

at least one occurrence of R₁ or R₂ is

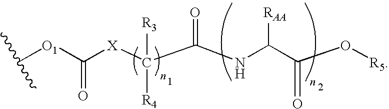

In a further embodiment of Formula 34, n₁ is an integer selected from 0 to 4.

In another Formula 34 embodiment, R₂ is

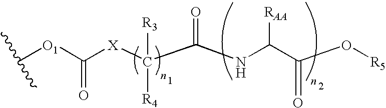

and each occurrence of R₁ is

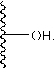

In one embodiment, R₁ on the benzene ring is

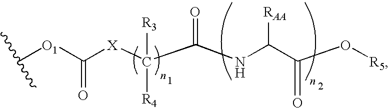

the other occurrence of R₁ is

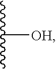

### R₂ is

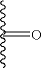

and n₁ is an integer selected from 0 to 4.

In one embodiment, X is absent from at least one prodrug moiety. In a further embodiment, X is absent from each prodrug moiety, if there are two prodrug moieties in the compound.

In one embodiment, R₁ is

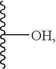

X is absent, n₁ is 0, 1, 2 or 3, n₂ is 1, 2 or 3 and R₃, R₄ and R₅ are each H. In a further embodiment, n₁ is 2. In one embodiment, R₁ is

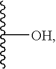

X is absent, n₁ is 0, 1, 2 or 3, n₂ is 1, 2, 3, 4 or 5 and R₃, R₄ and R₅ are each H. In a further embodiment, n₁ is 2.

In one embodiment, R₂ is

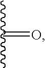

X is absent, n₁ is 0, 1, 2 or 3, n₂ is 1, 2, 3, 4 or 5 and R₃, R₄ and R₅ are each H. In a further embodiment, n₁ is 2. In one embodiment, R₁ is

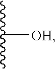

X is absent, n₁ is 0, 1, 2 or 3, n₂ is 1, 2, 3, 4 or 5 and R₃, R₄ and R₅ are each H. In a further embodiment, n₁ is 2.

In one embodiment, one occurrence of R₁ is

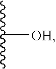

X is —O—, n₁ is 0, 1 or 2, n₂ is 1 or 2 and R₅ is H. In a further embodiment, n₁ is 2.

In one embodiment, R₂ is

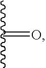

X is —NH—, n₁ is 0, 1 or 2, n₂ is 1 or 2 and R₅ is H. In a further embodiment, n₁ is 2.

In another embodiment, X is absent, n₁ is 1, 2 or 3 and n₂ is 1, 2 or 3.

In one embodiment, n₁ is 1 or 2 and n₂ is 1, 2, 3, 4 or 5. In a preferred embodiment, the oxymorphone prodrug of the present invention has one prodrug moiety, and the prodrug moiety has one or two amino acids (i.e., n₂ is 1 or 2). In one embodiment, the oxymorphone prodrug of the present invention has one prodrug moiety, and n₁ is 1 or 2 while n₂ is 1, 2 or 3.

In one embodiment, the oxymorphone compound has one prodrug moiety and, X is —O—, n₁ is 0, 1 or 2, n₂ is 1 or 2 and R₅ is H. In another oxymorphone embodiment, the compound has one prodrug moiety, X is —NH—, n₁ is 0, 1 or 2, n₂ is 1 or 2 and R₅ is H.

In a preferred embodiment, n₂ is 1, 2 or 3 while R₃, R₄ and R₅ are H. In another embodiment, n₂ is 1. In yet another embodiment, n₂ is 2. In yet another embodiment, n₂ is 1 or 2 and each occurrence of R_{AA} is independently a proteinogenic amino acid side chain.

In another oxymorphone embodiment, X is —O—, n₁ is 1, 2, 3 or 4, n₂ is 1, 2 or 3 and R₅ is H. In a further embodiment, at least one occurrence of R₃ is methyl.

In one oxymorphone embodiment, X is —NH—, n₁ is 0, 1 or 2, n₂ is 1 or 2 and R₅ is H.

In another oxymorphone embodiment, X is —NH—, n₁ is 1, 2, 3 or 4, n₂ is 1, 2 or 3 and R₅ is H. In a further embodiment, at least one occurrence of R₃ is methyl.

In yet another oxymorphone embodiment, X is absent, n₁ is 2, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon atom.

In one oxymorphone embodiment, X is absent, n₁ is 2, and one occurrence of R₃ or R₄ is —CH₃. In a further embodiment, R₅ is hydrogen.

In yet another oxymorphone embodiment, X is absent, n₁ is 3, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon.

In one oxymorphone embodiment, X is absent, n₁ is 2, and one occurrence of R₃ or R₄ is

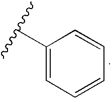

In a further embodiment, R₅ is hydrogen.

Another oxymorphone embodiment is directed to oxymorphone prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₅ is hydrogen. In even a further embodiment, the proteinogenic amino acid side chain is selected from valine, leucine and isoleucine.

Yet another oxymorphone embodiment is directed to oxymorphone prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₅ is hydrogen.

Itaconic acid, ketoglutaric and 2-methylene glutaric acid can also be used as a dicarboxylic acid linker in some oxymorphone embodiments. Here, R₃ and R₄ on one of the carbons defined by n₁, taken together, is a methylene group.

In one oxymorphone embodiment, the oxymorphone prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another oxymorphone embodiment includes oxymorphone prodrugs linked to a peptide or amino acid through a dicarboxylic acid linker substituted with an acetyl

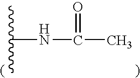

group or a carboxylic acid group. In a further oxymorphone embodiment, n₁ is 2 or 3 and R₃ is hydrogen. In even a further embodiment, the dicarboxylic acid linker is further substituted with an

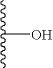

group.

In one embodiment, oxymorphone is linked to a peptide or prodrug through a citric acid linker. The citric acid linker can be any one of 6 isomers, as provided herein in Table 2.

In one embodiment, the oxymorphone prodrug of the present invention uses a dicarboxylic acid disclosed in Table 1 or 2 as the dicarboxylic acid linker

In another embodiment, R₅ is an opioid, and the oxymorphone and the additional opioid are linked via citroyl acid linker. In this embodiment, the additional carboxylic acid in the citroyl acid linker is bound to an amino acid or peptide. In a further embodiment, the additional opioid R₅ is oxymorphone.

In a further embodiment, the oxymorphone prodrug of the present invention is selected from an oxymorphone prodrug of Formula 35, 36, 37, 38, 39, 40, 41, 42 or 43, or a pharmaceutically acceptable salt thereof. For Formulae 35-43, O₁, R₃, R₄, R₅, n₁ and n₂ are defined as provided for Formula 34.

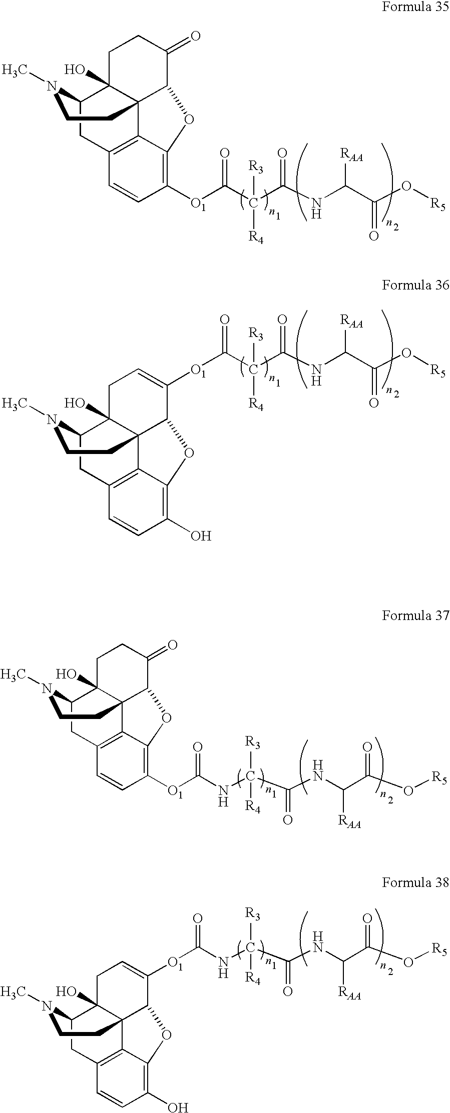

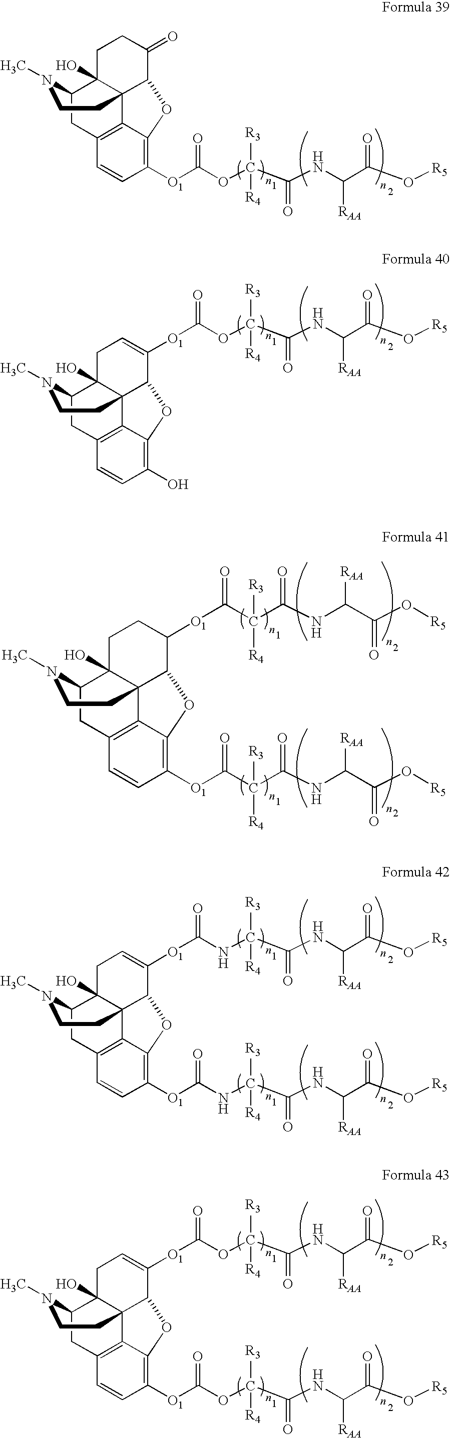

In a further Formulae 35-43, the —N— atom in the oxymorphone prodrug is demethylated.

Preferred embodiments of the oxymorphone prodrugs of the present invention are prodrugs wherein the side chain comprises a non-polar or an aliphatic amino acid, including the single amino acid prodrug oxymorphone succinyl valine ester, shown below.

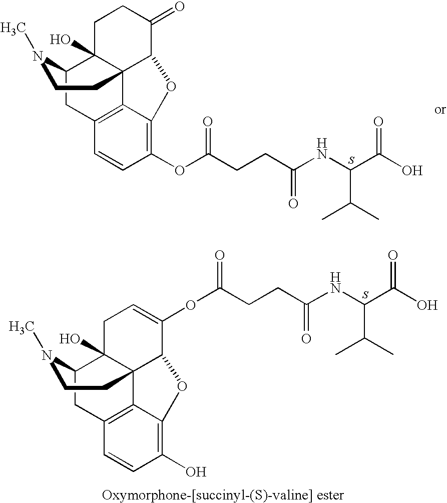

Other single amino acid prodrugs of oxymorphone include oxymorphone-[succinyl-(S)-isoleucine]ester, oxymorphone-[succinyl-(S)-leucine]ester, oxymorphone-[succinyl-(S)-aspartic acid] ester, oxymorphone-[succinyl-(S)-methionine]ester, oxymorphone-[succinyl-(S)-histidine]ester, oxymorphone-[succinyl-(S)-tyrosine]ester and oxymorphone-[succinyl-(S)-serine]ester.

In a preferred oxymorphone dipeptide embodiment, the present invention is directed to the dipeptide prodrugs oxymorphone-[succinyl-(S)-valine-valine]ester, oxymorphone-[succinyl-(S)-isoleucine-isoleucine]ester and oxymorphone-[succinyl-(S)-leucine-leucine]ester.

In another embodiment, oxymorphone prodrug moiety permutations can be drawn from valine, leucine, isoleucine, alanine and glycine. Yet further embodiments may include permutations drawn from these nonpolar aliphatic amino acids with the nonpolar aromatic amino acids, tryptophan and tyrosine.

Additionally, non-proteinogenic amino acid may also be used as the prodrug moiety in a oxymorphone prodrug, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above for the oxymorphone prodrug compounds are all in the L configuration. However, the present invention also contemplates oxymorphone prodrugs comprised of amino acids in the D configuration, or mixtures of amino acids in the D and L configurations.

In a preferred oxymorphone embodiment, the dicarboxylic acid linker is derived from succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the oxymorphone to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. The linkers provided in Tables 1 and 2 may also be used with oxymorphone prodrugs of the present invention, for example to conjugate the amino acid valine to oxymorphone.

### Meptazinol Prodrugs of the Present Invention

In one embodiment of the present invention, the prodrugs are novel amino acid and peptide prodrugs of meptazinol and are represented by Formula 44.

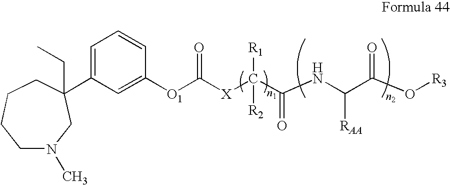

or a pharmaceutically acceptable salt thereof,

wherein,

O₁ is the phenolic oxygen atom present in the unbound meptazinol;

X is (—NH—), (—O—), or absent;

Each occurrence of R₁ and R₂ is independently selected from hydrogen, alkoxy,

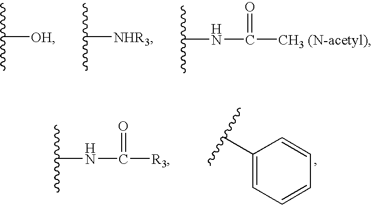

carboxyl, cycloalkyl, substituted cycloalkyl, alkyl, and substituted alkyl;

R₁ and R₂ on adjacent carbons can form a ring and R₁ and R₂ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

In the case of a double bond in the carbon chain defined by n₁, R₁ is present and R₂ is absent on the carbons that form the double bond;

R₃ is independently selected from hydrogen, alkyl, substituted alkyl, and an opioid;

When R₃ is an opioid, the —O— is a hydroxylic oxygen present in the additional opioid R₃; and

Each occurrence of R_{AA} is independently selected from a proteinogenic or non-proteinogenic amino acid side chain.

In a further embodiment of Formula 44, n₁ is an integer selected from 0 to 4.

In another embodiment, X is absent and n₁ is 1, 2 or 3. In a further embodiment, X is absent, n₁ is 1, 2 or 3, n₂ is 1 or 2 and R₁, R₂ and R₃ are each hydrogen.

In one embodiment, n₁ is 1, 2 or 3 and n₂ is 1, 2 or 3.

In one embodiment, n₂ is 1, 2, 3, 4 or 5. In a preferred embodiment, the prodrug moiety of a meptazinol compound of the present invention has one or two amino acids (i.e., n₂ is 1 or 2). In one embodiment, n₁ is 1 or 2 while n₂ is 1, 2 or 3.

In one embodiment, X is —O—, n₁ is 0, 1 or 2, n₂ is 1 or 2 and R₃ is H. In a further embodiment, at least one occurrence of R₁ is

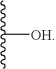

In another meptazinol embodiment, X is —NH—, n₁ is 0, 1 or 2, n₂ is 1 or 2 and R₃ is H. In a further embodiment, at least one occurrence of R₁ is

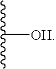

In a preferred embodiment, n₂ is 1, 2 or 3 while R₁, R₂ and R₃ are H. In another embodiment, n₂ is 1. In yet another embodiment, n₂ is 2. In yet another embodiment, n₂ is 1 or 2 and each occurrence of R_{AA} is independently a proteinogenic amino acid side chain.

In one embodiment, the compound of Formula 44 provides at least 10% greater oral bioavailability of meptazinol when compared to meptazinol administered alone.

Preferred embodiments of the meptazinol prodrugs of Formula 44 are prodrugs wherein the side chain comprises a non-polar or an aliphatic amino acid. One such prodrug, meptazinol-[succinyl-(S)-valine]ester, is represented below.

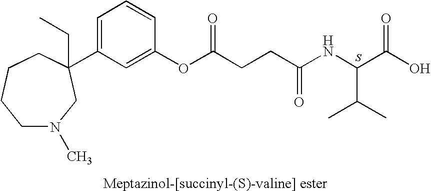

In another meptazinol embodiment, X is —O—, n₁ is 1, 2, 3 or 4, n₂ is 1, 2 or 3 and R₃ is H. In a further embodiment, at least one occurrence of R₁ is methyl.

In one meptazinol embodiment, X is —NH—, n₁ is 0, 1 or 2, n₂ is 1 or 2 and R₃ is H.

In another meptazinol embodiment, X is —NH—, n₁ is 1, 2, 3 or 4, n₂ is 1, 2 or 3 and R₃ is H. In a further embodiment, at least one occurrence of R₁ is methyl.

In yet another meptazinol embodiment, X is absent, n₁ is 2, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one meptazinol embodiment, X is absent, n₁ is 2, and one occurrence of R₁ or R₂ is —CH₃. In a further embodiment, R₃ is hydrogen.

In yet another meptazinol embodiment, X is absent, n₁ is 3, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one meptazinol embodiment, X is absent, n₁ is 2, and one occurrence of R₁ or R₂ is

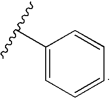

In a further embodiment, R₃ is hydrogen.

Another meptazinol embodiment is directed to opioid prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₃ is hydrogen. In even a further embodiment, the proteinogenic amino acid side chain is selected from valine, leucine and isoleucine.

Yet another meptazinol embodiment is directed to opioid prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₃ is hydrogen.

Itaconic acid, ketoglutaric and 2-methylene glutaric acid can also be used as a dicarboxylic acid linker in some meptazinol embodiments. Here, R₁ and R₂ on one of the carbons defined by n₁, taken together, is a methylene group.

In one meptazinol embodiment, the opioid prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another meptazinol embodiment includes opioid prodrugs linked to a peptide or amino acid through a dicarboxylic acid linker substituted with an acetyl

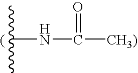

group or a carboxylic acid group. In a further embodiment, n₁ is 2 or 3 and R₃ is hydrogen. In even a further embodiment, the dicarboxylic acid linker is further substituted with an

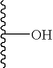

group.

In one meptazinol embodiment, meptazinol is linked to a peptide or prodrug through a citric acid linker. The citric acid linker can be any one of 6 isomers, as provided herein in Table 2.

In one embodiment, the meptazinol prodrug of the present invention uses a dicarboxylic acid disclosed in Table 1 or 2 as the dicarboxylic acid linker

In another embodiment, R₃ is an opioid, and meptazinol and the additional opioid are linked via citroyl acid linker. In this embodiment, the additional carboxylic acid in the citroyl acid linker is bound to an amino acid or peptide. In a further embodiment, the additional opioid R₃ is meptazinol.

The preferred amino acids for use in the present invention are in the L configuration. However, the present invention also contemplates prodrugs of Formula 44 comprised of amino acids in the D configuration, or mixtures of amino acids in the D and L configurations.

In one embodiment, prodrugs of Formula 44 can include prodrug moieties comprising one or more of the following amino acids—valine, leucine, isoleucine, alanine, and glycine. Further embodiments can include prodrug permutations drawn from these and other nonpolar aliphatic amino acids, with the nonpolar aromatic amino acids, tryptophan and tyrosine.

In one embodiment, a non-proteinogenic amino acid may be used as a prodrug moiety of the present invention (or portion thereof), either as either a single amino acid, included in a dipeptide or another short peptide. In the peptide embodiments, the peptide can contain only non-proteinogenic amino acids, or a combination of proteinogenic and non-proteinogenic amino acids.

Meptazinol is attached to the amino acid or short peptide through a dicarboxylic acid linker, e.g., malonic, succinic, glutaric, adipic, or other longer chain dicarboxylic acid linker or substituted derivatives thereof.

A preferred dicarboxylic acid linker is derived from succinic acid. Single amino acid prodrugs using this linker include meptazinol-[succinyl-(S)-isoleucine]ester, meptazinol-[succinyl-(S)-leucine]ester, meptazinol-[succinyl-(S)-aspartic acid] ester, meptazinol-[succinyl-(S)-methionine]ester, meptazinol-[succinyl-(S)-histidine]ester, meptazinol-[succinyl-(S)-tyrosine]ester and meptazinol-[succinyl-(S)-serine]ester.

Preferred dipeptide prodrugs of meptazinol using the dicarboxylic acid linker include meptazinol-[succinyl-(S)-valine-valine]ester, meptazinol-[succinyl-(S)-isoleucine-isoleucine]ester and meptazinol-[succinyl-(S)-leucine-leucine]ester.

As alternatives to the use of an unsubstituted dicarboxylic acid linker to attach the opioid to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic.

Examples of dicarboxylic acid linkers that can be used with meptazinol are given in Tables 1 and 2.

Representative valine prodrugs of meptazinol are given in Table 7. These examples are not meant to limit the scope of meptazinol prodrugs encompassed by the present invention. Valine can be readily substituted with other single amino acids or peptides to form other dicarboxylic acid linked meptazinol prodrugs.

TABLE 7
Non-Limiting Examples of Meptazinol Prodrugs of the Present Invention

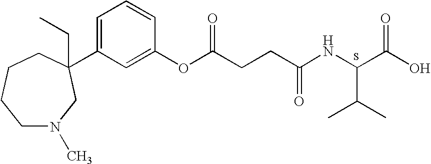

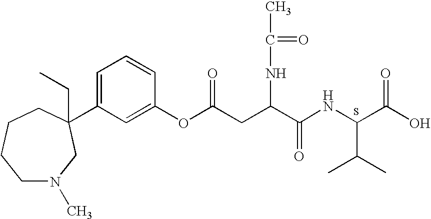

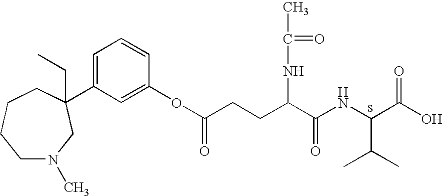

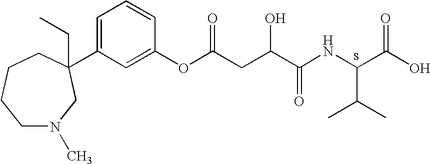

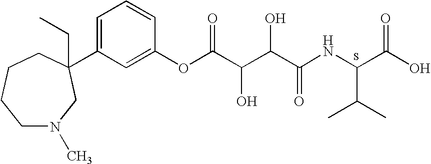

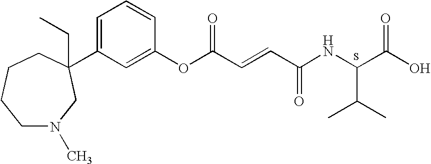

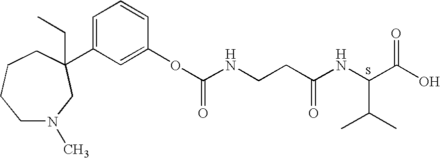

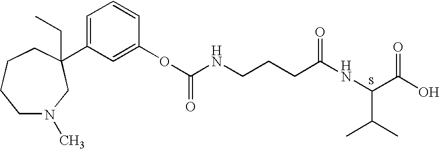

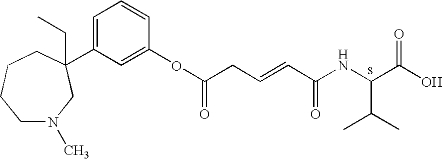

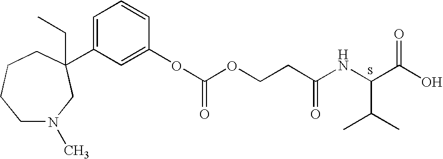

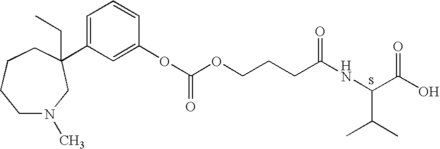

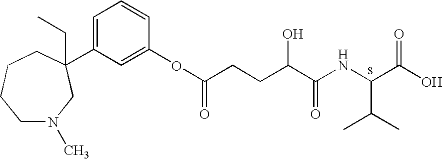

The present invention also contemplates meptazinol prodrugs where a meptazinol metabolite is employed (e.g., ethyl-hydroxylated meptazinol (3-[3-(2-Hydroxy-ethyl)-1-methyl-perhydro-azepin-3-yl]-phenol), (3-[3-(2-carboxy-ethyl)-1-methyl-perhydro-azepin-3-yl]-phenol), des-methyl meptazinol, 2-oxomeptazinol and 7-oxomeptazinol). Therefore, in one embodiment, the present invention is directed to meptazinol and meptazinol metabolite prodrugs of Formula 44(a). In Formula 44(a) embodiments, O₁, X, R₁, R₂, R₃, R_{AA}, n₁ and n₂ are defined as provided for Formula 44.

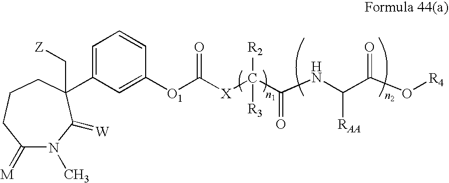

or a pharmaceutically acceptable salt thereof, wherein,

A is selected from O and S,

M and W are independently O or absent, and only one of M and W can be present on any one molecule,

Z is methyl, CH₂OH or COOH,

R₁ is H or methyl,

if Z is CH₂OH or COOH, M and W are both absent and R₁ is methyl,

if M or W is present, Z and R₁ are both methyl,

if R₁ is H, M and W are both absent while Z is methyl,

O₁ is the phenolic oxygen atom present in the unbound meptazinol;

X is (—NH—), (—O—), or absent;

Each occurrence of R₂ and R₃ is independently selected from hydrogen, alkoxy,

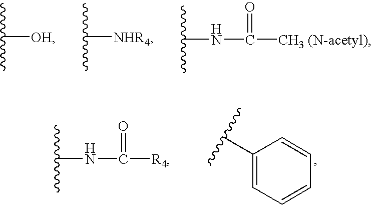

carboxyl, cycloalkyl, substituted cycloalkyl, alkyl, and substituted alkyl;

R₂ and R₃ on adjacent carbons can form a ring and R₂ and R₃ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

In the case of a double bond in the carbon chain defined by n₁, R₁ is present and R₂ is absent on the carbons that form the double bond;

R₄ is independently selected from hydrogen, alkyl, substituted alkyl and an opioid;

When R₄ is an opioid, the —O— is a hydroxylic oxygen present in the additional opioid R₄; and

Each occurrence of R_{AA} is independently selected from a proteinogenic or non-proteinogenic amino acid side chain.

In a further embodiment of Formula 44(a), n₁ is an integer selected from 0 to 4.

In another Formula 44(a) embodiment, X is absent and n₁ is 1, 2 or 3. In a further embodiment, X is absent, n₁ is 1, 2 or 3, n₂ is 1 or 2 and R₁, R₂ and R₃ are each hydrogen.

In one Formula 44(a) embodiment, n₁ is 1, 2 or 3 and n₂ is 1, 2 or 3.

In one embodiment, prodrugs of Formula 44(a) can include prodrug moieties comprising one or more of the following amino acids—valine, leucine, isoleucine, alanine, and glycine. Further embodiments can include prodrug permutations drawn from these and other nonpolar aliphatic amino acids, with the nonpolar aromatic amino acids, tryptophan and tyrosine.

Preferred embodiments of the N-demethylated meptazinol prodrugs of Formula 44(a) are prodrugs wherein the side chain comprises a non-polar or an aliphatic amino acid. One such prodrug is represented below.

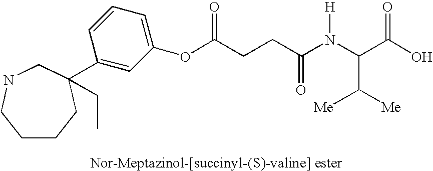

### Hydrocodone Prodrugs of the Present Invention

In one embodiment, the prodrugs of the present invention are directed to hydrocodone prodrugs of Formula 45, below.

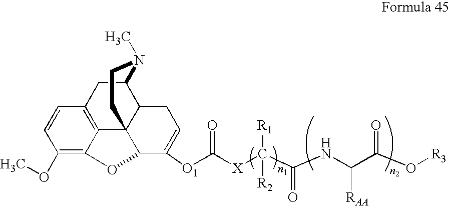

or a pharmaceutically acceptable salt thereof,

wherein,

O₁ is the enolized oxygen atom of hydrocodone;

X is (—NH—), (—O—), or absent;

Each occurrence of R₁ and R₂ is independently selected from hydrogen, alkoxy,

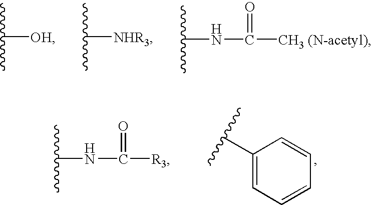

carboxyl, cycloalkyl, substituted cycloalkyl, alkyl, and substituted alkyl;

R₁ and R₂ on adjacent carbons can form a ring and R₁ and R₂ on the same carbon, taken together, can be a methylene group;

n₁ is an integer selected from 0 to 16 and n₂ is an integer selected from 1 to 9;

the carbon chain defined by n₁ can include a cycloalkyl or aromatic ring;

In the case of a double bond in the carbon chain defined by n₁, R₁ is present and R₂ is absent on the carbons that form the double bond;

R₃ is independently selected from hydrogen, alkyl, substituted alkyl, and an opioid;

When R₃ is an opioid, the —O— is a hydroxylic oxygen present in the additional opioid R₃; and

Each occurrence of R_{AA} is independently selected from a proteinogenic or non-proteinogenic amino acid side chain.

In a further embodiment of Formula 45, n₁ is an integer selected from 0 to 4. In yet a further Formula 45 embodiment, the prodrug is N- or O-demethylated.

In another embodiment, X is absent and n₁ is 1, 2 or 3. In a further embodiment, X is absent, n₁ is 1, 2 or 3, n₂ is 1 or 2 and R₁, R₂ and R₃ are each hydrogen. In yet a further embodiment, the prodrug is N- or O-demethylated.

In one embodiment, X is absent, n₁ is 1, 2 or 3 and n₂ is 1, 2 or 3. In a further embodiment at least one occurrence of R₁ is,

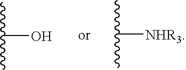

In yet a further embodiment, the prodrug is N- or O-demethylated.

In one embodiment, X is —O—, n₁ is 0, 1 or 2, n₂ is 1 or 2 and R₅ is H. In another hydrocodone embodiment, X is —NH—, n₁ is 0, 1 or 2, n₂ is 1 or 2 and R₅ is H. In a further embodiment, at least one occurrence of R₁ is,

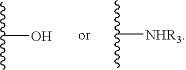

In yet a further embodiment, the prodrug is N- or O-demethylated.

In one embodiment, n₂ is 1, 2, 3, 4 or 5. In a preferred embodiment, the prodrug moiety of a hydrocodone compound of the present invention has one or two amino acids (i.e., n₂ is 1 or 2). In one embodiment, n₁ is 1 or 2 while n₂ is 1, 2 or 3. In a further embodiment at least one occurrence of R₁ is,

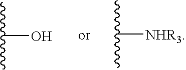

In yet a further embodiment, the prodrug is N- or O-demethylated.

In a preferred embodiment, n₂ is 1, 2 or 3 while R₃, R₄ and R₅ are H. In another embodiment, n₂ is 1. In yet another embodiment, n₂ is 2. In yet another embodiment, n₂ is 1 or 2 and each occurrence of R_{AA} is independently a proteinogenic amino acid side chain. In a further embodiment each occurrence of R₃, R₄ and R₅ are H. In yet a further embodiment, the prodrug is N- or O-demethylated.

In one embodiment, the compound of Formula 45 provides at least 10% greater oral bioavailability of hydrocodone when compared to hydrocodone administered alone. In a further embodiment, the prodrug is N- or O-demethylated.

In a preferred hydrocodone embodiment, the present invention is directed to hydrocodone prodrugs that include a non-polar or aliphatic amino acid, including the single amino acid prodrug hydrocodone-[succinyl-(S)-valine] enol ester, shown below.

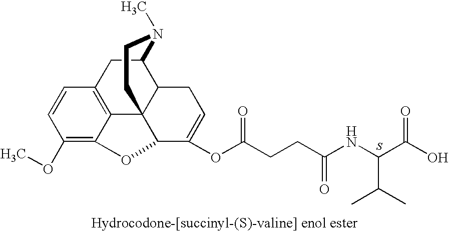

In a preferred embodiment, the single amino acid prodrug of hydrocodone is the trifluoroacetate salt of hydrocodone-[succinyl-(S)-valine] enol, shown below.

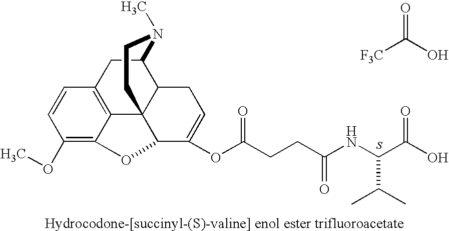

Other single amino acid prodrugs of hydrocodone include hydrocodone-[succinyl-(S)-isoleucine] enol ester, hydrocodone-[succinyl-(S)-leucine] enol ester, hydrocodone-[succinyl-(S)-aspartic acid] enol ester, hydrocodone-[succinyl-(S)-methionine] enol ester, hydrocodone-[succinyl-(S)-histidine] enol ester, hydrocodone-[succinyl-(S)-tyrosine] enol ester and hydrocodone-[succinyl-(S)-serine] enol ester. In another embodiment, the hydrocodone prodrugs of the present invention are either O- or N-demethylated.

In a preferred hydrocodone dipeptide embodiment, the present invention is directed to the dipeptide prodrugs hydrocodone-[succinyl-(S)-valine-valine] enol ester, hydrocodone-[succinyl-(S)-isoleucine-isoleucine] enol ester and hydrocodone-[succinyl-(S)-leucine-leucine] enol ester. In yet a further embodiment, the aforementioned prodrugs are either N- or O-demethylated.

In another hydrocodone embodiment, X is —O—, n₁ is 1, 2, 3 or 4, n₂ is 1, 2 or 3 and R₃ is H. In a further embodiment, at least one occurrence of R₁ is methyl.

In one hydrocodone embodiment, X is —NH—, n₁ is 0, 1 or 2, n₂ is 1 or 2 and R₃ is H.

In another hydrocodone embodiment, X is —NH—, n₁ is 1, 2, 3 or 4, n₂ is 1, 2 or 3 and R₃ is H. In a further embodiment, at least one occurrence of R₁ is methyl.

In yet another hydrocodone embodiment, X is absent, n₁ is 2, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one hydrocodone embodiment, X is absent, n₁ is 2, and one occurrence of R₁ or R₂ is —CH₃. In a further embodiment, R₃ is hydrogen.

In yet another hydrocodone embodiment, X is absent, n₁ is 3, one occurrence of R₁ is —CH₃, and one occurrence of R₂ is —CH₃. In a further embodiment, R₃ is hydrogen. In still a further embodiment, the one occurrence of R₁ and R₂ groups that are methyl occur on the same carbon.

In one hydrocodone embodiment, X is absent, n₁ is 2, and one occurrence of R₁ or R₂ is

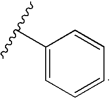

In a further embodiment, R₃ is hydrogen.

Another hydrocodone embodiment is directed to opioid prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₃ is hydrogen. In even a further embodiment, the proteinogenic amino acid side chain is selected from valine, leucine and isoleucine.

Yet another hydrocodone embodiment is directed to opioid prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₃ is hydrogen.

Itaconic acid, ketoglutaric and 2-methylene glutaric acid can also be used as a dicarboxylic acid linker in some hydrocodone embodiments. Here, R₁ and R₂ on one of the carbons defined by n₁, taken together, is a methylene group.

In one hydrocodone embodiment, the opioid prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another hydrocodone embodiment includes opioid prodrugs linked to a peptide or amino acid through a dicarboxylic acid linker substituted with an acetyl

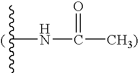

group or a carboxylic acid group. In a further embodiment, n₁ is 2 or 3 and R₃ is hydrogen. In even a further embodiment, the dicarboxylic acid linker is further substituted with an

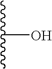

group.

In one embodiment, hydrocodone is linked to a peptide or prodrug through a citric acid linker. The citric acid linker can be any one of 6 isomers, as provided herein in Table 2.

In one embodiment, the hydrocodone prodrug of the present invention uses a dicarboxylic acid disclosed in Table 1 or 2 as the dicarboxylic acid linker

In another embodiment, R₃ is an opioid, and hydrocodone and the additional opioid are linked via citroyl acid linker. In this embodiment, the additional carboxylic acid in the citroyl acid linker is bound to an amino acid or peptide. In a further embodiment, the additional opioid R₃ is hydrocodone.

Further embodiments may include permutations drawn from these nonpolar aliphatic amino acids with the nonpolar aromatic amino acids, tryptophan and tyrosine.

Additionally, non-proteinogenic amino acid may also be used as the prodrug moiety, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above are all in the L configuration. However, the present invention also contemplates hydrocodone prodrugs comprised of amino acids in the D configuration, or mixtures of amino acids in the D and L configurations.

In a preferred embodiment, the dicarboxylic acid linker is succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the opioid to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Examples of linkers for use with hydrocodone are given in Tables 1 and 2.

Nalbuphine Prodrugs of the Present Invention

In one embodiment, prodrugs of the present invention are directed to novel nalbuphine prodrugs of Formula 46, below.

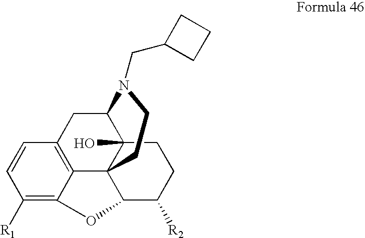

a pharmaceutically acceptable salt thereof,

wherein,

R₁ and R₂ are independently selected from

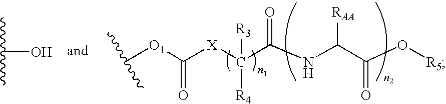

Each occurrence of O₁ is independently an oxygen atom present in the unbound form of nalbuphine;

Each occurrence of X is independently (—NH—), (—O—), or absent;

Each occurrence of R₃ and R₄ is independently selected from hydrogen, alkoxy,

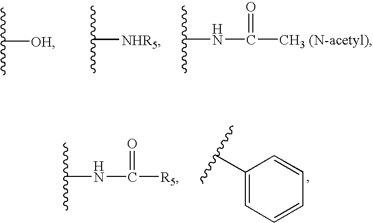

carboxyl, cycloalkyl, substituted cycloalkyl, alkyl, and substituted alkyl;

R₃ and R₄ on adjacent carbons can form a ring and R₃ and R₄ on the same carbon, taken together, can be a methylene group;

Each occurrence of n₁ is independently an integer selected from 0 to 16 and each occurrence of n₂ is independently an integer selected from 1 to 9;

the carbon chain defined by n₁ can include a cycloalkyl or aromatic ring;

In the case of a double bond in the carbon chain defined by n₁, R₃ is present and R₄ is absent on the carbons that form the double bond;

Each occurrence of R₅ is independently selected from hydrogen, alkyl, substituted alkyl group and an opioid;

When R₅ is an opioid, the —O— is a hydroxylic oxygen present in the additional opioid R₅;

Each occurrence of R_{AA} is independently selected from a proteinogenic or non-proteinogenic amino acid side chain; and

at least one of R₁ and R₂ is

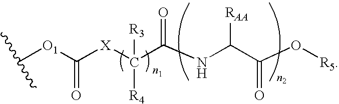

In a further Formula 46 embodiment, n₁ is an integer selected from 0 to 4. In yet a further embodiment, the nalbuphine prodrug is N-dealkylated.

In another Formula 46 embodiment, R₂ is

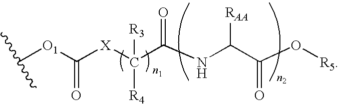

In a further embodiment, X is absent and n₁ is 1, 2 or 3. In yet a further embodiment, the nalbuphine prodrug is N-dealkylated.

In one embodiment, R₁ is

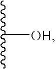

X is absent, n₁ is 0, 1, 2 or 3, n₂ is 1, 2 or 3 and R₃, R₄ and R₅ are each H. In a further embodiment, n₁ is 2. In another embodiment, R₁ is

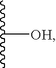

X is absent, n₁ is 0, 1, 2 or 3, n₂ is 1, 2, 3, 4 or 5 and R₃, R₄ and R₅ are each H. In a further embodiment, n₁ is 2. In yet a further embodiment, the nalbuphine prodrug is N-dealkylated.

In one embodiment, R₂ is

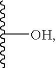

X is absent, n₁ is 0, 1, 2 or 3, n₂ is 1, 2 or 3 and R₃, R₄ and R₅ are each H. In a further embodiment, n₁ is 2. In another embodiment, R₂ is

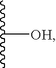

X is absent, n₁ is 0, 1, 2 or 3, n₂ is 1, 2, 3, 4 or 5 and R₃, R₄ and R₅ are each H. In a further embodiment, n₁ is 2. In yet a further embodiment, the nalbuphine prodrug is N-dealkylated.

In one embodiment, R₁ is

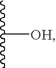

X is —O—, n₁ is 0, 1 or 2, n₂ is 1 or 2 and R₅ is H. In a further embodiment, n₁ is 2. In one embodiment, R₁ is

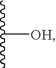

X is —NH—, n₁ is 0, 1 or 2, n₂ is 1 or 2 and R₅ is H. In a further embodiment, n₁ is 2. In yet a further embodiment, the nalbuphine prodrug is N-dealkylated.

In one embodiment, R₂ is

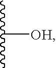

X is —O—, n₁ is 0, 1, 2 or 3, n₂ is 1 or 2 and R₅ is H. In a further embodiment, n₁ is 2. In one embodiment, R₂ is

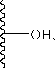

X is —NH—, n₁ is 0, 1, 2 or 3, n₂ is 1 or 2 and R₅ is H. In a further embodiment, n₁ is 2. In yet a further embodiment, the nalbuphine prodrug is N-dealkylated.

In one embodiment, X is absent and n₁ is 1, 2 or 3 and n₂ is 1, 2 or 3. In one embodiment, X is absent n₁ is 1 or 2 and n₂ is 1, 2, 3, 4 or 5. In a further embodiment, the nalbuphine prodrug is N-dealkylated.

In one embodiment, R₁ is

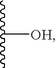

n₁ is 1, 2 or 3, n2 is 1 or 2 and at least one occurrence of R₃ is

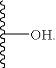

In one embodiment, R₂ is

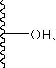

n₁ is 1, 2 or 3, n₂ is 1 or 2 and at least one occurrence of R₃ is

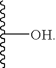

In a further embodiment, the nalbuphine prodrug is N-dealkylated.

In a preferred embodiment, the nalbuphine prodrug of the present invention has one prodrug moiety, and the prodrug moiety has one or two amino acids (i.e., n₂ is 1 or 2). In one embodiment, the nalbuphine prodrug of the present invention has one prodrug moiety, and n₁ is 1 or 2 while n₂ is 1, 2 or 3. In a further embodiment, the nalbuphine prodrug is N-dealkylated.

In a preferred embodiment, n₂ is 1, 2 or 3 while R₃, R₄ and R₅ are H. In another embodiment, n₂ is 1. In yet another embodiment, n₂ is 2. In yet another embodiment, n₂ is 1 or 2 and each occurrence of R_{AA} is independently a proteinogenic amino acid side chain. In a further embodiment, the nalbuphine prodrug is N-dealkylated.

In another nalbuphine embodiment, X is —O—, n₁ is 1, 2, 3 or 4, n₂ is 1, 2 or 3 and R₅ is H. In a further embodiment, at least one occurrence of R₃ is methyl.

In one nalbuphine embodiment, X is —NH—, n₁ is 0, 1 or 2, n₂ is 1 or 2 and R₅ is H.

In another nalbuphine embodiment, X is —NH—, n₁ is 1, 2, 3 or 4, n₂ is 1, 2 or 3 and R₅ is H. In a further embodiment, at least one occurrence of R₃ is methyl.

In yet another nalbuphine embodiment, X is absent, n₁ is 2, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon atom.

In one nalbuphine embodiment, X is absent, n₁ is 2, and one occurrence of R₃ or R₄ is —CH₃. In a further embodiment, R₅ is hydrogen.

In yet another nalbuphine embodiment, X is absent, n₁ is 3, one occurrence of R₃ is —CH₃, and one occurrence of R₄ is —CH₃. In a further embodiment, R₅ is hydrogen. In still a further embodiment, the one occurrence of R₃ and R₄ groups that are methyl occur on the same carbon.

In one nalbuphine embodiment, X is absent, n₁ is 2, and one occurrence of R₃ or R₄ is

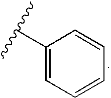

In a further embodiment, R₅ is hydrogen.

Another nalbuphine embodiment is directed to nalbuphine prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, R₅ is hydrogen. In even a further embodiment, the proteinogenic amino acid side chain is selected from valine, leucine and isoleucine.

Yet another nalbuphine embodiment is directed to nalbuphine prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioc acid and E-methoxybutenedioic acid. In a further embodiment, R₅ is hydrogen.

Itaconic acid, ketoglutaric and 2-methylene glutaric acid can also be used as a dicarboxylic acid linker in some nalbuphine embodiments. Here, R₃ and R₄ on one of the carbons defined by n₁, taken together, is a methylene group.

In one nalbuphine embodiment, the nalbuphine prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker (n₁ is 6 in both cases).

Still, another nalbuphine embodiment includes nalbuphine prodrugs linked to a peptide or amino acid through a dicarboxylic acid linker substituted with an acetyl

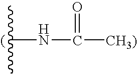

group or a carboxylic acid group. In a further nalbuphine embodiment, n₁ is 2 or 3 and R₃ is hydrogen. In even a

further embodiment, the dicarboxylic acid linker is further substituted with an

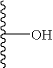

group.

In one embodiment, nalbuphine is linked to a peptide or prodrug through a citric acid linker. The citric acid linker can be any one of 6 isomers, as provided herein in Table 2.

In one embodiment, the nalbuphine prodrug of the present invention uses a dicarboxylic acid disclosed in Table 1 or 2 as the dicarboxylic acid linker

In another embodiment, R₅ is an opioid, and nalbuphine and the additional opioid are linked via citroyl acid linker. In this embodiment, the additional carboxylic acid in the citroyl acid linker is bound to an amino acid or peptide. In a further embodiment, the additional opioid R₅ is nalbuphine.

In a further embodiment, the nalbuphine prodrug of the present invention is selected from an nalbuphine prodrug of Formulae 48, 49, 50, 51, 52, 53, 54, and 55, or a pharmaceutically acceptable salt thereof. For Formulae 48-56, O₁, R₃, R₄, R₅, n₁ and n₂ are defined as given for Formula 46.

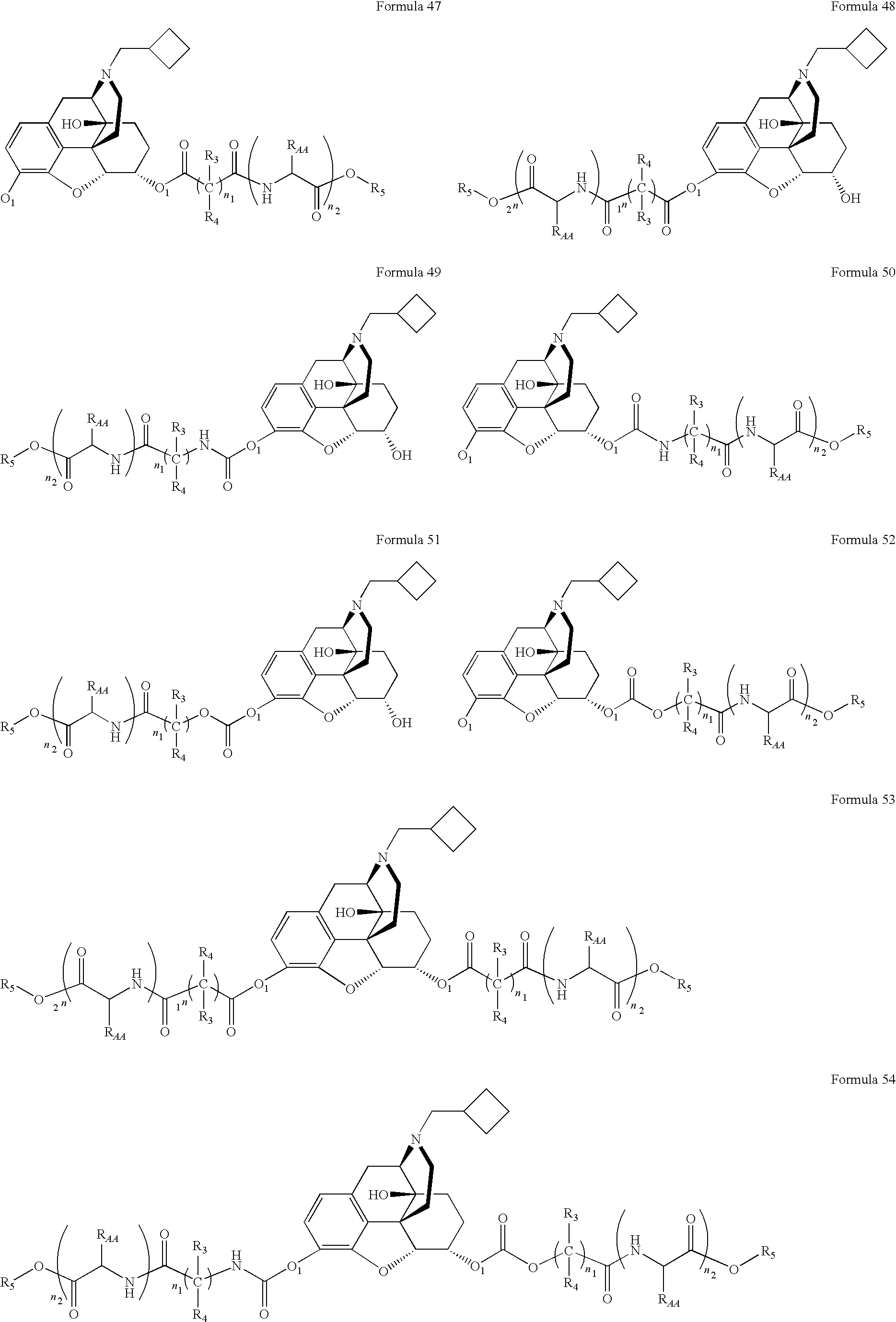

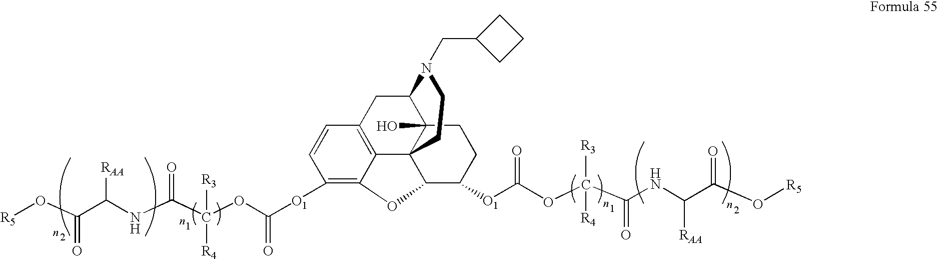

In a further Formulae 47-55 embodiment, the nalbuphine prodrug is N-dealkylated.

Still, in another embodiment, the nalbuphine prodrug can have two prodrug moieties, wherein X is present in one, but absent in the other (not shown in the above formulae).

Preferred embodiments of the nalbuphine prodrugs of the present invention are prodrugs wherein the side chain comprises a non-polar or an aliphatic amino acid, including the single amino acid prodrug nalbuphine succinyl valine ester, shown below.

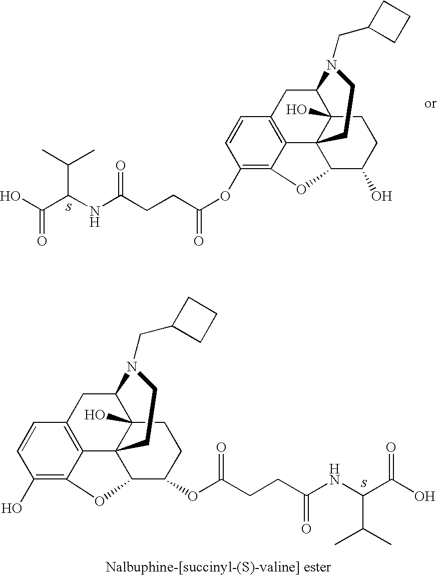

Other single amino acid prodrugs of nalbuphine include nalbuphine-[succinyl-(S)-isoleucine]ester, nalbuphine-[succinyl-(S)-leucine]ester, nalbuphine-[succinyl-(S)-aspartic acid] ester, nalbuphine-[succinyl-(S)-methionine]ester, nalbuphine-[succinyl-(S)-histidine]ester, nalbuphine-[succinyl-(S)-tyrosine]ester and nalbuphine-[succinyl-(S)-serine]ester. In a further embodiment of the present invention, the prodrugs listed above are N-dealkylated.

In a preferred nalbuphine dipeptide embodiment, the present invention is directed to the dipeptide pro drugs nalbuphine-[succinyl-(S)-valine-valine]ester, nalbuphine-[succinyl-(S)-isoleucine-isoleucine]ester and nalbuphine-[succinyl-(S)-leucine-leucine]ester. In a further embodiment of the present invention, the prodrugs listed above are N-dealkylated.

In another embodiment, nalbuphine prodrug moiety permutations can be drawn from valine, leucine, isoleucine, alanine and glycine. Yet further embodiments may include permutations drawn from these nonpolar aliphatic amino acids with the nonpolar aromatic amino acids, tryptophan and tyrosine.

Additionally, non-proteinogenic amino acid may also be used as the prodrug moiety in a nalbuphine prodrug, either as a single amino acid or part of a peptide. A peptide that includes a non-proteinogenic amino acid may contain only non-proteinogenic amino acids, or a combination of proteinogenic and non-proteinogenic amino acids.

The preferred amino acids described above for the nalbuphine prodrug compounds are all in the L configuration. However, the present invention also contemplates nalbuphine prodrugs comprised of amino acids in the D configuration, or mixtures of amino acids in the D and L configurations.

In a preferred nalbuphine embodiment, the dicarboxylic acid linker is derived from succinic acid. Other dicarboxylic acid linkers within the scope of the invention include, but are not limited to, malonic acid, glutaric acid, adipic acid, or other longer chain dicarboxylic acids or substituted derivatives thereof.

As alternatives to the use of a dicarboxylic acid linker to attach the nalbuphine to the amino acid or peptide prodrug moiety, other substituted dicarboxylic acid linkers may be employed. For example, methyl malonic acid may be used. Such substituted dicarboxylic acid linkers would preferably be naturally occurring in the subject to be treated, i.e., non-xenobiotic. Examples of dicarboxylic acid linkers for use with the nalbuphine prodrugs of the present invention are given in Tables 1 and 2. These can be conjugated to an amino acid or short peptide, for example, valine.

Advantages of the Compounds of the Invention

Without wishing to be bound to any particular theory, it is believed that the amino acid or peptide portion of the opioid prodrug of the present invention (e.g., the amino acid or peptide portion of any of Formulae 1-55) selectively exploits the inherent di- and tripeptide transporter Pept1 within the digestive tract to effect absorption of the drug. It is believed that the opioid analgesic is subsequently released from the amino acid or peptide prodrug by hepatic and extrahepatic hydrolases that are in part, present in plasma.

Furthermore, the prodrugs of the present invention (for example, prodrugs of Formulae 1-55) temporarily reduce the respective opioid binding properties of the parent compound, minimizing any potential for local opioid action within the gut lumen on opioid or other receptors. Once absorbed, however, the opioid prodrug of the present invention is metabolized by plasma and liver esterases to the pharmacologically active opioid species, which can then elicit its centrally mediated analgesic effects.

Reduction of the adverse GI side-effects associated with opioid administration may also be an added advantage of using a prodrug of the present invention. Oral administration of a temporarily inactivated opioid would, during the absorption process, preclude access of active drug species to the μ-opioid receptors within the gut wall. The role that these peripheral μ-opioid receptors play on gut transit has recently been demonstrated by co-administration of peripherally confined narcotic antagonists such as alvimopan, methylnaltrexone and naloxone. (Linn and Steinbrook (2007). Tech in Reg. Anaes. and Pain Management 11, 27-32). Co-administration of these active agents with normally constipating opioid analgesics such as oxycodone has shown a reduction in effects on gut transit, without adversely affecting systemically mediated analgesia. Thus, oral administration of a transiently inactivated opioid may similarly avoid such problems of locally mediated constipation, without the need for co-administration of a peripheral μ-opioid antagonist.

Improvement in the pharmacokinetics of the opioids described herein is another advantage of a prodrug of the present invention. Oral administration of a prodrug of the present invention affords temporary protection against the possibility of extensive first pass metabolism and the consequential low bioavailability, and resultant variability, in attained plasma drug levels. Such temporary shielding of the metabolically vulnerable phenolic or hydroxylic function by a prodrug moiety should ensure reduced first pass metabolism of the drug and improve the oral bioavailability of the respective opioid. Additionally, the administration of a prodrug could also lead to maintenance of drug in plasma as the result of continuing generation of drug from a plasma reservoir of prodrug.

The improvements in bioavailability offered by the prodrugs of the present invention are likely to lead to greater predictability of analgesic response both within and between subjects (potential for less variability of analgesic response and drug plasma levels for both (1) individual subjects and (2) a subject population) and hence improve subject compliance.

Another potential advantage of the prodrugs presented herein is a reduced likelihood of intravenous or intranasal abuse. An initially inactive opioid prodrug may reduce the propensity for intravenous abuse because of the prodrug's slower attainment rate of peak active drug levels, compared to administration of free opioid. This should give a reduced “euphoric rush” to potential abusers. Intranasal abuse may also be reduced by the greater likelihood of poor absorption of a hydrophilic prodrug via the nasal mucosa. This would be the consequence of the profound difference in physicochemical properties between the parent opioid and a highly water soluble amino acid or peptide prodrug described herein. Amino acid/peptide prodrugs are not likely to be absorbed by simple diffusion due to their high water solubility and also adverse LogP values. Instead, they would rely upon active transporters, such as Pept1, which, while present in the gut, are essentially absent in the nasal mucosa.

### USES AND METHODS OF THE INVENTION

One embodiment of the present invention is a method of treating a disorder in a subject in need thereof with an opioid. The method comprises orally administering a therapeutically effective amount (e.g., an analgesic effective amount) of an opioid prodrug of the present invention to the subject, or a pharmaceutically acceptable salt thereof (e.g., a prodrug of any of Formulae 1-55). The disorder may be one treatable with an opioid. For example, the disorder may be pain, such as neuropathic pain or nociceptive pain. Specific types of pain which can be treated with the opioid prodrugs of the present invention include, but are not limited to, acute pain, chronic pain, post-operative pain, pain due to neuralgia (e.g., post herpetic neuralgia or trigeminal neuralgia), pain due to diabetic neuropathy, dental pain, pain associated with arthritis or osteoarthritis, and pain associated with cancer or its treatment. Any of the prodrugs presented herein can be used in a method of treating pain.

In the methods of treating pain, the prodrugs encompassed by the present invention may be administered in conjunction with other therapies and/or in combination with other active agents (e.g., other analgesics). For example, the prodrugs encompassed by the present invention may be administered to a subject in combination with other active agents used in the management of pain. An active agent to be administered in combination with the prodrugs encompassed by the present invention may include, for example, a drug selected from the group consisting of non-steroidal anti-inflammatory drugs (e.g., ibuprofen), anti-emetic agents (e.g., ondansetron, domerperidone, hyoscine and metoclopramide), and unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent. The prodrug and other active agent(s) may also be incorporated into a single dosage form.

In one embodiment, the present invention is directed to a method for minimizing the gastrointestinal side effects normally associated with administration of an opioid analgesic, wherein the opioid has a derivatizable group. The method comprises orally administering an opioid prodrug or a pharmaceutically acceptable salt thereof to a subject in need thereof, wherein the opioid prodrug is comprised of an opioid analgesic covalently bonded via a dicarboxylic acid linker, to an amino acid or peptide of 2-9 amino acids in length, and wherein upon oral administration, the prodrug or pharmaceutically acceptable salt minimizes, if not completely avoids, the gastrointestinal side effects usually seen after oral administration of the unbound opioid analgesic. The amount of the opioid is preferably a therapeutically effective amount (e.g., an analgesic effective amount). According to one preferred embodiment, the opioid prodrug includes the same opioid as the discontinued opioid analgesic. The term “unbound opioid analgesic” refers to an opioid analgesic which is not a prodrug. This method is particularly useful for reducing gastrointestinal side effect(s) resulting from or aggravated by administration of the unbound opioid analgesic for pain relief. In this embodiment, the opioid prodrug can be any opioid prodrug of Formulae 1-55 or a pharmaceutically acceptable salt thereof. In a further embodiment, the opioid prodrug can be selected from any succinyl-valine ester presented herein.

In another embodiment, the present invention is directed to a method for increasing the oral bioavailability of an opioid analgesic which has a significantly lower bioavailability when administered alone. The method comprises administering, to a subject in need thereof, an opioid prodrug or a pharmaceutically acceptable salt thereof to a subject in need thereof, wherein the opioid prodrug is comprised of an opioid analgesic covalently bonded via a dicarboxylic acid linker, to an amino acid or peptide of 2-9 amino acids in length, and wherein upon oral administration, the oral bioavailability of the opioid derived from the prodrug is at least twice that of the opioid, when administered alone. The amount of the opioid is preferably a therapeutically effective amount (e.g., an analgesic effective amount). In this embodiment, the opioid prodrug can be any opioid prodrug of Formulae 1-55 or a pharmaceutically acceptable salt thereof. In a further embodiment, the opioid prodrug can be selected from any succinyl-valine ester presented herein.

### Salts, Solvates, and Derivatives of the Compounds of the Invention

The compounds, compositions and methods of the present invention further encompass the use of salts, solvates, of the opioid prodrugs described herein. In one embodiment, the invention disclosed herein is meant to encompass all pharmaceutically acceptable salts of opioid prodrugs (including those of the carboxyl terminus of the amino acid as well as those of the weakly basic morphinan nitrogen).

Typically, a pharmaceutically acceptable salt of a prodrug of an opioid of the present invention is prepared by reaction of the prodrug with a desired acid or base, as appropriate. The salt may precipitate from solution and be collected by filtration or may be recovered by evaporation of the solvent. For example, an aqueous solution of an acid such as hydrochloric acid may be added to an aqueous suspension of the opioid prodrug and the resulting mixture evaporated to dryness (lyophilized) to obtain the acid addition salt as a solid. Alternatively, the prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain (1999). Polymorphism in Pharmaceutical solids. Marcel Decker, New York). The compounds useful in the practice of the present invention can have one or more chiral centers and, depending on the nature of individual substituents, they can also have geometrical isomers.

### Pharmaceutical Compositions of the Invention

While it is possible that, for use in the methods of the invention, the prodrug of the present invention may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice. In one embodiment of the present invention, a composition comprising an opioid prodrug of the present invention (e.g., a prodrug of any of Formulae 1-34) is provided. The composition comprises at least one opioid prodrug selected from Formula 1-34, and at least one pharmaceutically acceptable excipient or carrier.

The formulations of the invention may be immediate-release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, e.g., in Remington, The Science and Practice of Pharmacy, 20^{th} Edition, 2000, pp. 858-914.

However since absorption of amino acid and peptide pro-drugs of opioids may proceed via an active transporter such as Pept1, controlled dosage forms may be desirable. As the Pept1 transporter is believed to be largely confined to the upper GI tract this may limit the opportunity for continued absorption along the whole length of the GI tract.

For those opioid prodrugs which do not result in sustained plasma drugs levels due to continuous generation of active agent from a plasma reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption take place and effectively prolonging plasma drug levels. Other drug delivery systems affording delayed progression along the GI tract may also be of value.

The formulations of the present invention can be administered from one to six times daily, depending on the dosage form and dosage.

In one embodiment, the present invention provides a pharmaceutical composition comprising at least one active pharmaceutical ingredient (i.e., an opioid prodrug), or a pharmaceutically acceptable derivative (e.g., a salt or solvate) thereof, and a pharmaceutically acceptable carrier or excipient. In particular, the invention provides a pharmaceutical composition comprising a therapeutically effective amount of at least one opioid prodrug of the present invention, or a pharmaceutically acceptable derivative thereof, and a pharmaceutically acceptable carrier or excipient.

The prodrug employed in the present invention may be used in combination with other therapies and/or active agents. Accordingly, the present invention provides, in another embodiment, a pharmaceutical composition comprising at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds are preferably stable in the presence of, and compatible with each other and the other components of the formulation. When formulated separately, they may be provided in any convenient formulation, conveniently in such manner as are known for such compounds in the art.

The prodrugs presented herein may be formulated for administration in any convenient way for use in human or veterinary medicine. The invention therefore includes pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, see, e.g., International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia, cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyan

The pharmaceutical compositions of the invention may contain from 0.01 to 99% weight per volume of the prodrugs encompassed by the present invention.

### Dosages

The doses referred to throughout the specification refer to the amount of the opioid free base equivalents in the particular compound, unless otherwise specified.

Appropriate patients to be treated according to the methods of the invention include any human or animal in need of such treatment. Methods for the diagnosis and clinical evaluation of pain, including the severity of the pain experienced by an animal or human are well known in the art. Thus, it is within the skill of the ordinary practitioner in the art (e.g., a medical doctor or veterinarian) to determine if a patient is in need of treatment for pain. The patient is preferably a mammal, more preferably a human, but can be any subject or animal, including a laboratory animal in the context of a clinical trial, screening, or activity experiment employing an animal model. Thus, as can be readily appreciated by one of ordinary skill in the art, the methods and compositions of the present invention are particularly suited to administration to any animal or subject, particularly a mammal, and including, but not limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending on the severity of pain to be treated, a suitable therapeutically effective and safe dosage, as may readily be determined within the skill of the art, can be administered to subjects. For oral administration to humans, the daily dosage level of the prodrug may be in single or divided doses. The duration of treatment may be determined by one of ordinary skill in the art, and should reflect the nature of the pain (e.g., a chronic versus an acute condition) and/or the rate and degree of therapeutic response to the treatment. Typically, a physician will determine the actual dosage which will be most suitable for an individual subject.

The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents such as buprenorphine, the daily dose requirement may, for example, range from 0.5 to 50 mg, preferably from 1 to 25 mg, and more preferably from 1 mg to 10 mg. For less potent agents such as meptazinol, the daily dose requirement may, for example, range from 1 mg to 1600 mg, preferably from 1 mg to 800 mg and more preferably from 1 mg to 400 mg.

In the methods of treating pain, the prodrugs encompassed by the present invention may be administered in conjunction with other therapies and/or in combination with other active agents. For example, the prodrugs encompassed by the present invention may be administered to a patient in combination with other active agents used in the management of pain. An active agent to be administered in combination with the prodrugs encompassed by the present invention may include, for example, a drug selected from the group consisting of non-steroidal anti-inflammatory drugs (e.g., acetaminophen and ibuprofen), anti-emetic agents (e.g., ondanstron, domerperidone, hyoscine and metoclopramide), unabsorbed or poorly bioavailable opioid antagonists to reduce the risk of drug abuse (e.g., naloxone). In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical composition. For example, a prodrug encompassed by the present invention and another active agent may be administered in a substantially simultaneous manner, such as in a single capsule or tablet having a fixed ratio of these agents, or in multiple separate dosage forms for each agent.

When the prodrugs of the present invention are used in combination with another agent active in the methods for treating pain, the dose of each compound may differ from that when the compound is used alone. Appropriate doses will be readily appreciated by those of ordinary skill in the art.

### EXAMPLES

The present invention is further illustrated by reference to the following Examples. However, it should be noted that these Examples, like the embodiments described above, are illustrative and are not to be construed as restricting the enabled scope of the invention in any way.

Preparation of the Prodrugs of the Present Invention

Compounds employed in the present invention may be prepared by the general methods provided herein.

Chemicals were purchased primarily from Aldrich Chemical Company, Gillingham, Dorset and Alfa Aesar, Morecambe, Lancashire, U.K. and were used without further purification Anhydrous solvents were used. Gasoline employed was the fraction boiling in the range 40-60° C.

TLC was carried out using aluminum plates pre-coated with silica gel (Kieselgel 60 F₂₅₄, 0.2 mm, Merck, Darmstadt, Germany). Visualization was by UV light or KMnO₄ dip. Silica gel (‘flash’, Kieselgel 60) was used for medium pressure chromatography.

¹H NMR spectra were recorded on a Bruker Avance BVT3200 spectrometer using deuterated solvents as internal standards.

Combustion analyses were performed by Advanced Chemical and Material Analysis, Newcastle University, U.K. using a Carlo-Erba 1108 elemental analyzer.

The methods taught in U.S. Provisional Patent Application No. 61/211,831 and 61/227,716 are incorporated herein by reference in their entireties.

### Example 1

### General Route of Synthesis for Amino Acid or Peptide Dicarboxylic Acid Conjugates of Opioids

Two general routes of synthesis to dicarboxylic acid linked amino acid or peptide conjugates of opioids as their HCl or TFA salts are given in Scheme 1 (alcohol ester) and 2 (enol ester) below. These routes of synthesis are illustrated using a succinic acid linker. This can, however, be applied to all dicarboxylic acid linkers of the present invention.

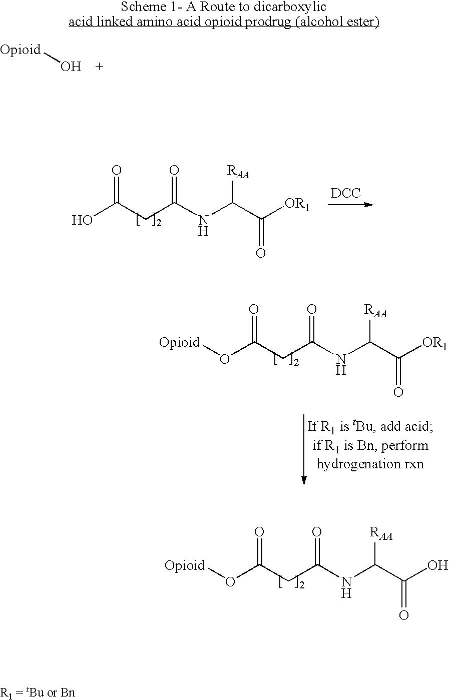

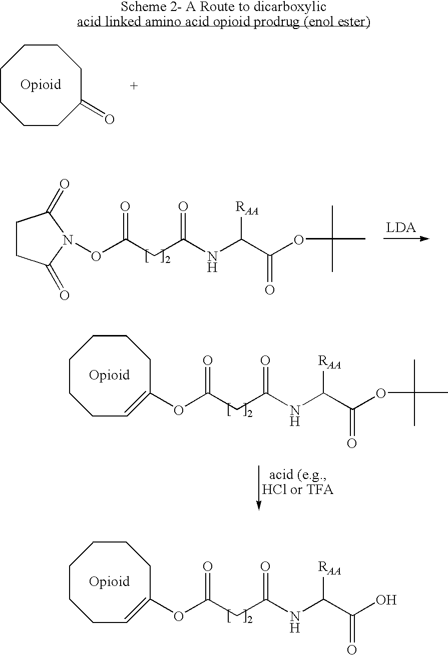

The compounds listed in Table 8, using meptazinol and valine as examples of a hydroxylic opioid and amino acid, respectively, can be made by these methods. It is to be understood that other opioids can be readily substituted for meptazinol, for conjugation to the various prodrug moieties described herein. One of ordinary skill in the art will also readily know how to substitute another amino acid or peptide, where desired.

TABLE 8
Non-Limiting Meptazinol Prodrugs of the Present Invention.

| | Prodrug | Structure |
|---|---|---|
| 1 | Meptazinol-(R)-2-methylsuccinic acid-linked valine Isomer 1 (S)-Isomer also within the scope of the present invention | 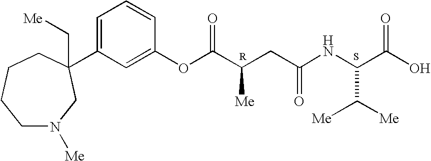 |
| 2 | Meptazinol-(R)-2-methylsuccinic acid-linked valine Isomer 2 (S)-Isomer also within the scope of the present invention | 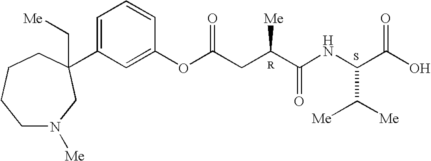 |
| 3 | Meptazinol 2,2-Dimethylsuccinic acid-linked valine Isomer 1 | 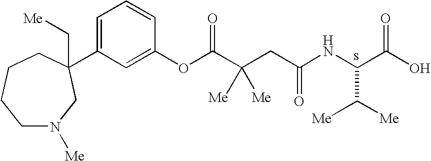 |
| 4 | Meptazinol 2,2-Dimethylsuccinic acid-linked valine Isomer 2 | 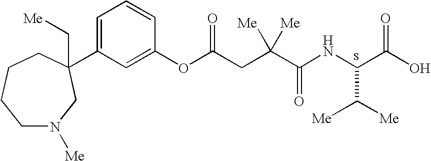 |
| 5 | Meptazinol 2,3-Dimethylsuccinic acid-linked valine Mixture of isomers | 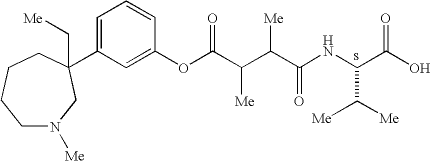 |
| 6 | Meptazinol-(R)-2-phenylsuccinic acid-linked valine Isomer 1 (S)-Isomer also within the scope of the present invention | 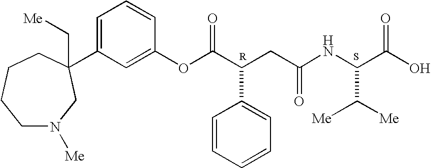 |
| 7 | Meptazinol-(R)-2-phenylsuccinic acid-linked valine Isomer 2 (S)-Isomer also within the scope of the present invention | 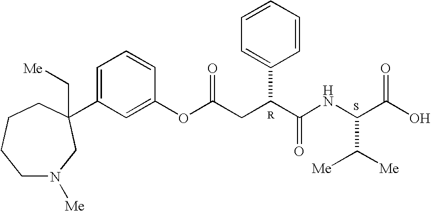 |
| 8 | Meptazinol 2,2-Dimethylglutaric acid-linked valine Isomer 1 | 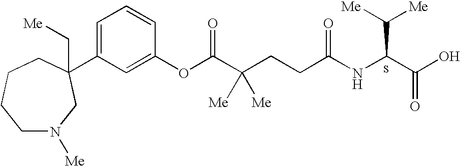 |
| 9 | Meptazinol 2,2-Dimethylglutaric acid-linked valine Isomer 2 | 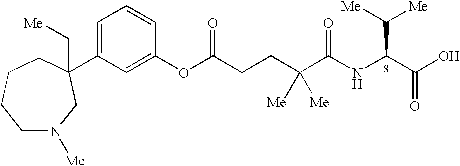 |
| 10 | Meptazinol 3,3-Dimethylglutaric acid-linked valine | 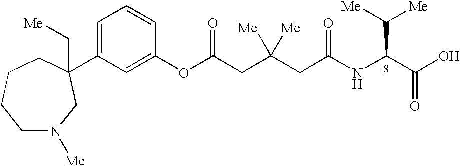 |
| 11 | Meptazinol Maleic acid-linked valine | 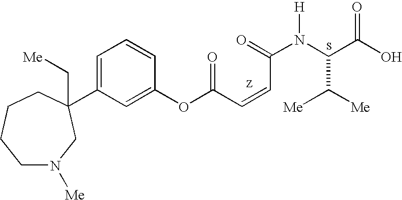 |
| 12 | Mepazinol fumaric acid linked valine | 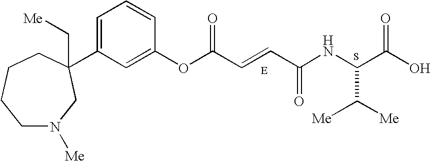 |
| 13 | Meptazinol-citraconic acid-linked valine Isomer 1 | 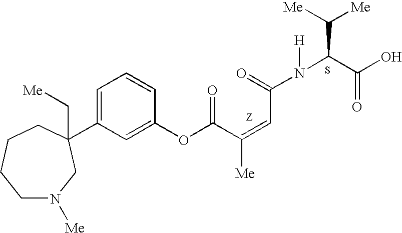 |
| 14 | Meptazinol-citraconic acid-linked valine Isomer 2 | 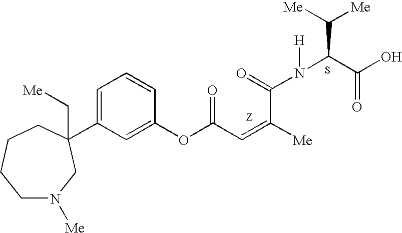 |
| 15 | Meptazinol 3,3-Dimethylmaleic acid-linked valine | 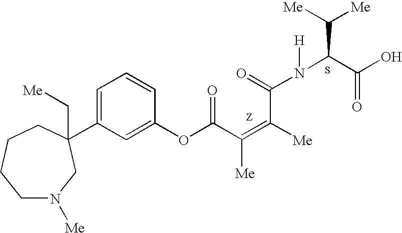 |
| 16 | Mepazinol 2,3-dimethylfumaric acid linked valine | 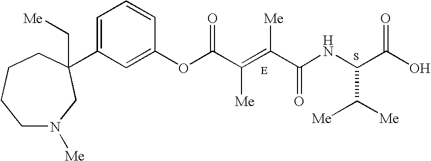 |
| 17 | Meptazinol Z-Methoxybutenedioic acid-linked valine Isomer 1 | 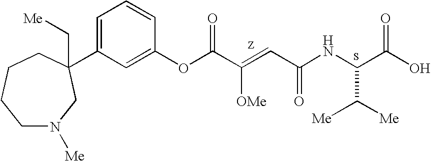 |
| 18 | Meptazinol Z-Methoxybutenedioic acid-linked valine Isomer 2 | 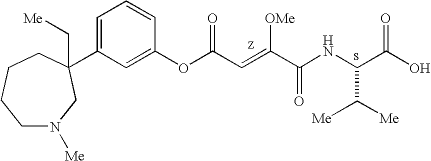 |
| 19 | Meptazinol E-Methoxybutenedioic acid-linked valine Isomer 2 | 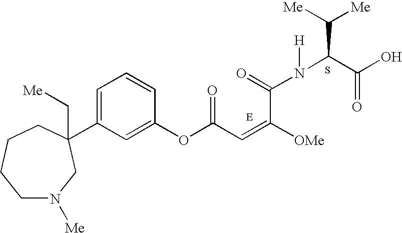 |
| 20 | Meptazinol itaconic acid-linked valine Isomer 1 | 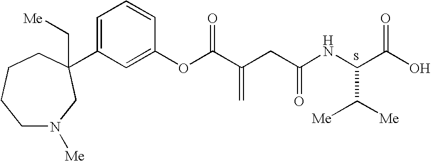 |
| 21 | Meptazinol itaconic acid-linked valine Isomer 2 | 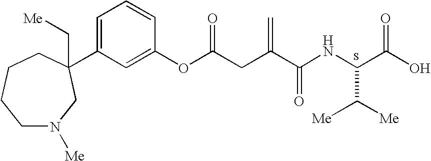 |
| 22 | Meptazinol 2-methylene-glutaric acid-linked valine Isomer 1 | 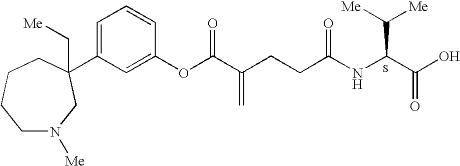 |
| 23 | Meptazinol 2-methylene-glutaric acid-linked valine Isomer 2 | 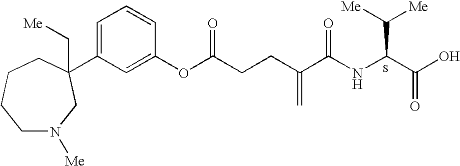 |
| 24 | Meptazinol (E)-glutaconic acid-linked valine Isomer 1 | 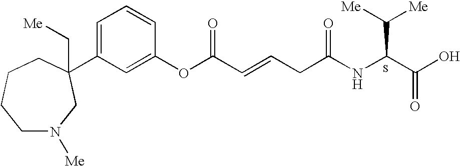 |
| 25 | Meptazinol (E)-glutaconic acid-linked valine Isomer 2 | 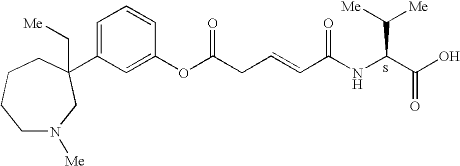 |
| 26 | Meptazinol phthalic acid-linked valine | 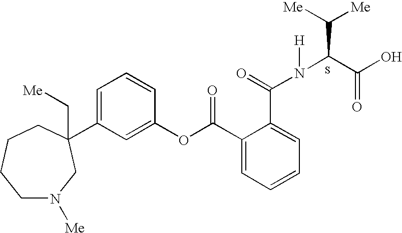 |
| 27 | Meptazinol terephthalic acid-linked valine | 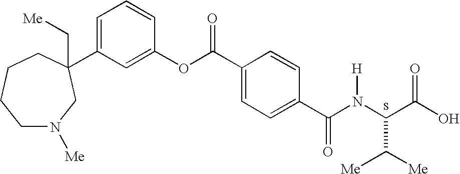 |
| 28 | Meptazinol malic acid-linked valine Isomer 1 | 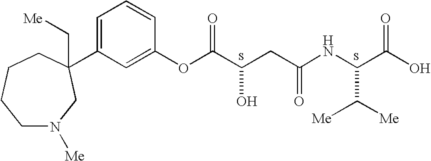 |
| 29 | Meptazinol malic acid-linked valine Isomer 2 | 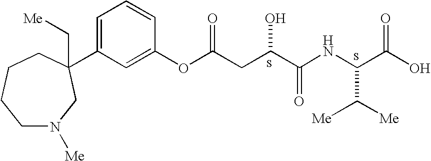 |
| 30 | Meptazinol tartaric acid-linked valine | 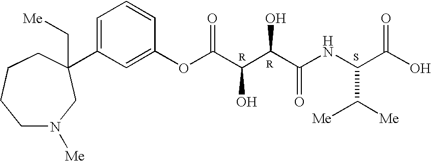 |
| 31 | Meptazinol (S)-citramalic acid-linked valine Isomer 1 | 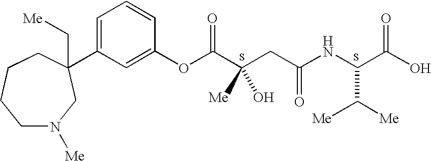 |
| 32 | Meptazinol (S)-citramalic acid-linked valine Isomer 2 | 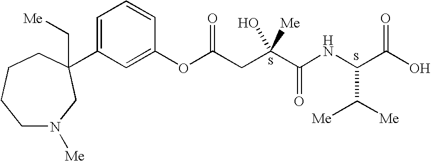 |
| 33 | Meptazinol aconitic acid-linked valine Isomer 1 | 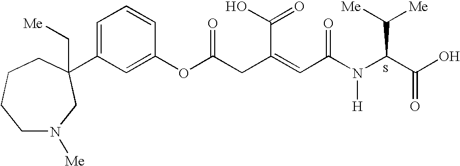 |
| 34 | Meptazinol aconitic acid-linked valine Isomer 2 | 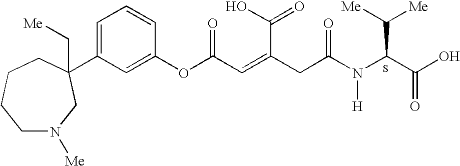 |
| 35 | Meptazinol α-Ketoglutaric acid-linked valine Isomer 1 | 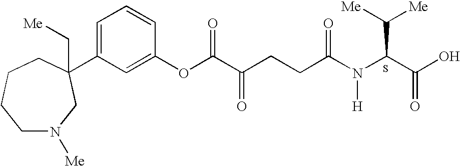 |
| 36 | Meptazinol α-Ketoglutaric acid-linked valine Isomer 2 | 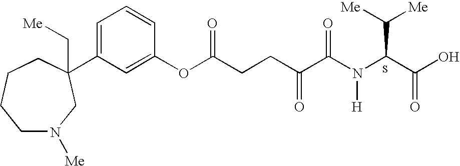 |
| 37 | Meptazinol N^{α}-Acetyl aspartic acid linked valine Isomer 1 | 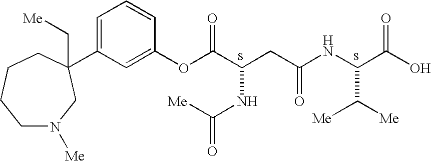 |
| 38 | Meptazinol N^{α}-Acetyl aspartic acid linked valine Isomer 2 | 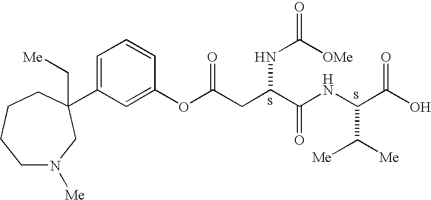 |
| 39 | Meptazinol N^{α}-Acetyl glutamatic acid linked valine Isomer 1 | 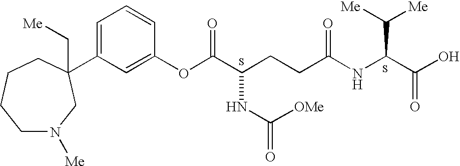 |
| 40 | Meptazinol N^{α}-Acetyl glutamatic acid linked valine Isomer 2 | 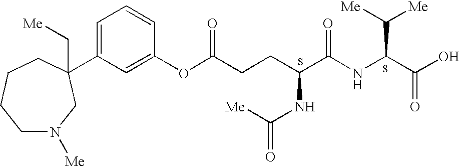 |
| 42 | Mepazinol γ-Aminobutyric acid (GABA) linked valine | 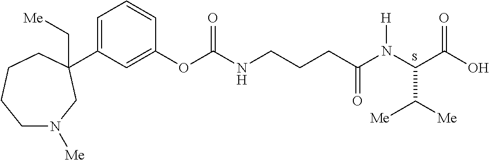 |
| 43 | Meptazinol (hydroxylpropionyl-valine) carbonate | 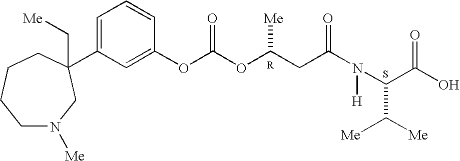 |
| 44 | (1,5-Di-meptazinol-citroyl)-valine | 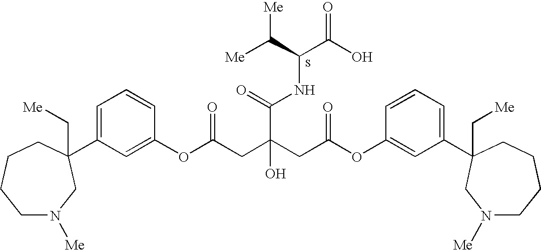 |
| 45 | Meptazinol isocitric acid-linked valine Isomer 1 | 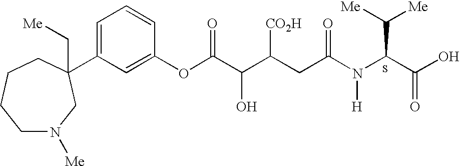 |
| 46 | Meptazinol isocitric acid-linked valine Isomer 2 | 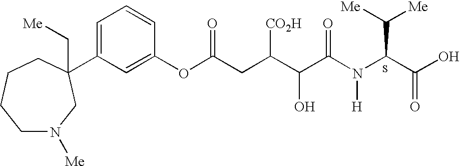 |
| 47 | Meptazinol (2R,3S)-2-hydroxy-3-methylsuccinic acid-linked valine Isomer 1 | 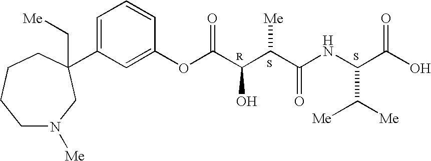 |
| 48 | Meptazinol (2R,3S)-2-hydroxy-3-methylsuccinic acid-linked valine Isomer 2 | 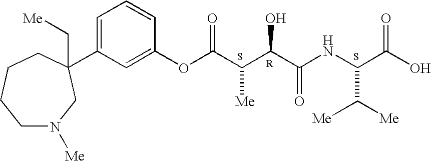 |
| 49 | Meptazinol (2R,3S)-2-hydroxy-2,3- dimethylsuccinic acid-linked valine Isomer 1 | 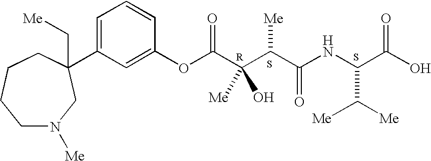 |
| 50 | Meptazinol (2R,3S)-2-hydroxy-2,3- dimethylsuccinic acid-linked valine Isomer 2 | 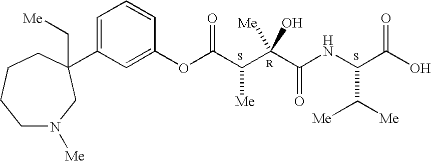 |
| 51 | Meptazinol citric acid-linked valine Isomer 1 | 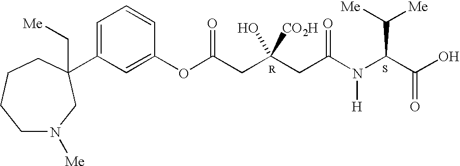 |
| 52 | Meptazinol citric acid-linked valine Isomer 2 | 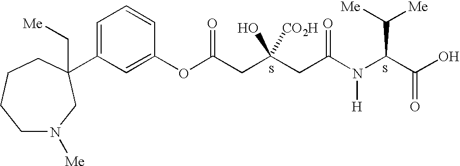 |
| 53 | Meptazinol citric acid-linked valine Isomer 3 | 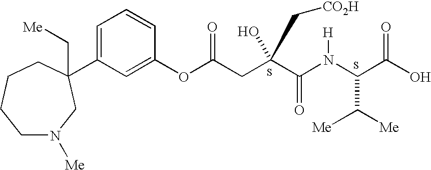 |
| 54 | Meptazinol citric acid-linked valine Isomer 4 | 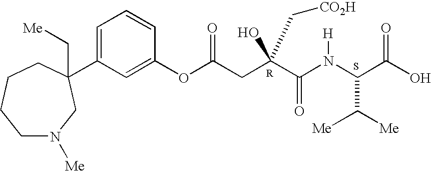 |
| 55 | Meptazinol citric acid-linked valine Isomer 5 | 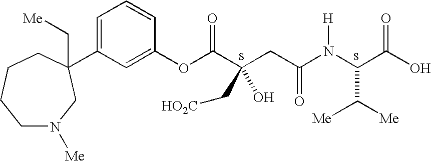 |
| 56 | Meptazinol citric acid-linked valine Isomer 6 | 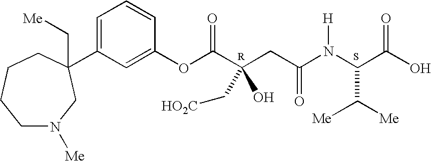 |

### Example 2

### Synthesis of Oxycodone-[Succinyl-(S)-Valine] Enol Ester

A general synthetic route to oxycodone-[succinyl-(S)-valine]ester is given in Scheme 3.

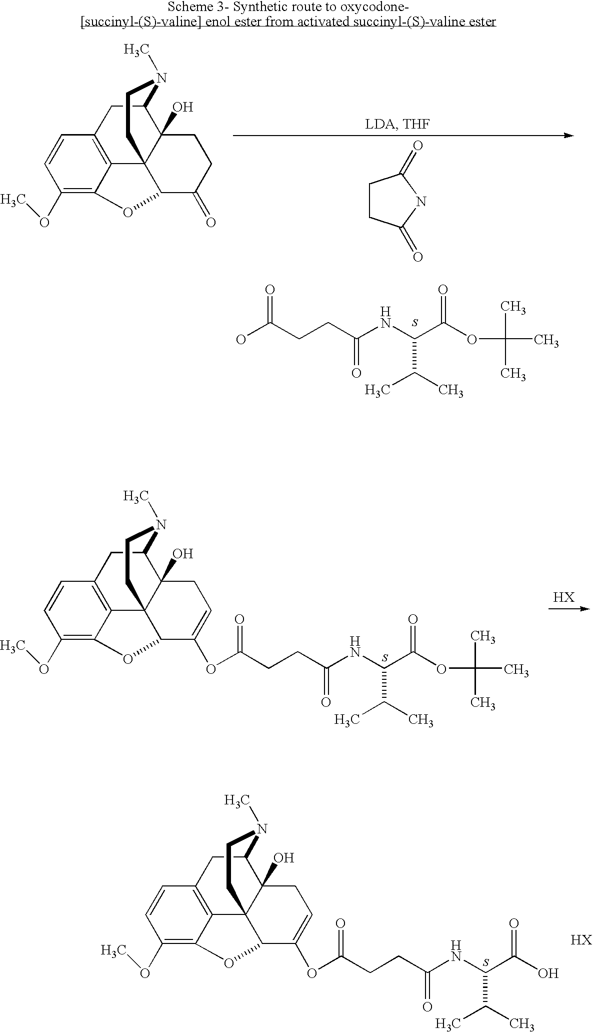

### Detailed Description of Synthesis of (N-Hydroxysuccinimidyl)-succinyl-(S)-valine-O-tert-butyl Ester

A solution of N,N-dicyclohexylcarbodi-imide (958 mg, 4.64 mmol) in ethyl acetate (15 mL) was added to succinyl-(S)-valine-O-tert-butyl ester (1.21 g, 4.42 mmol) and N-hydroxysuccinimide (560 mg, 4.86 mmol) in ethyl acetate (22 mL). The reaction was stirred at 50° C. for 2 hours. The resulting mixture was cooled to room temperature and filtered through celite. The filtrate was washed twice with saturated aqueous sodium bicarbonate solution (50 mL), water (50 mL) and brine (50 mL), dried (MgSO₄) and concentrated to give the required (N-hydroxysuccinimidyl)-succinyl-(S)-valine-O-tert-butyl ester (1.5 g, 92%), as a white solid.

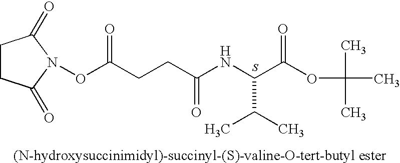

¹H NMR (CDCl₃, 300 MHz): δ 6.03 (d, J=8.1 Hz, 1H, NH), 4.40 (dd, J=8.7, 4.5 Hz, 1H, valine α-CH), 2.29 (m, 2H, succinyl CH₂), 2.76 (s, 4H, 2×succinimide CH₂), 2.60 (m, 2H, succinyl CH₂), 2.07 (m, 1H, valine β-CH), 1.30 (s, 9H, tert-butyl), 0.84 (t, J=7.2 Hz, 6H, 2×valine CH₃).

### Detailed Description of Synthesis of Oxycodone-[Succinyl-(S)-Valine] Ester Trifluoroacetate

To a solution of oxycodone free base (376 mg, 1.19 mmol) in tetrahydrofuran (18 mL) under nitrogen at 0° C. was added lithium di-isopropylamide (LDA) (1.8 M in tetrahydrofuran, heptane, ethylbenzene) (0.73 mL, 141 mg, 1.31 mmol). The reaction mixture was stirred at the same temperature for 30 minutes. (N-Hydroxysuccinimidyl)-succinyl-(S)-valine-O-tert-butyl ester (1.32 g, 3.58 mmol) was added in one portion to the cooled mixture and the reaction was allowed to warm to room temperature overnight. The resulting mixture was filtered through celite and concentrated to give a white foam. Flash chromatography (5→30% MeOH-diethyl ether) afforded a mixture 1:1 of oxycodone and oxycodone succinyl-(S)-valine tert-butyl ester (587 mg), as a white foam. The oxycodone was removed by treating the mixture with a solid-supported hydrazine derivative.

The mixture (587 mg) was dissolved in trifluoroacetic acid (7 mL) and the resulting solution was stirred at room temperature for 15 minutes. After this time, the mixture was evaporated and residual trifluoroacetic acid was removed under vacuum azeotropically by treatment with chloroform (5 times) to afford a white foam (680 mg). This foam was chromatographed by preparative HPLC and freeze-dried overnight to give oxycodone [succinyl-(S)-valine]ester trifluoroacetate (170 mg, 23% overall), as a white solid.

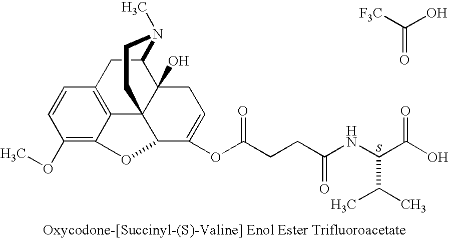

¹H NMR (DMSO-d₆) δ 12.58 (br s, 1H, COOH), 9.19 (br s, 1H, NH), 8.10 (d, J=8.4 Hz, 1H, NH), 6.87 (d, J=8.4 Hz, 1H, ArH), 6.75 (d, J=8.4 Hz, 1H, ArH), 6.29 (br s, 1H, OH), 5.51 (m, 1H, vinyl-H), 4.98 (s, 1H, 5-H), 4.15 (dd, J=8.4, 5.8 Hz, 1H, α-CH), 3.76 (s, 3H, OMe), 3.64 (d, J=5.7 Hz, 1H, ½ CH₂), 3.09 (m, 2H, CH₂), 2.84 (s, 3H, NMe), 2.63 (m, 2H, CH₂), 2.27 (dd, J=18, 5.4 Hz, 1H, ½ CH₂), 2.04 (m, 2H, β-CH and ½ CH₂), 1.63 (d, J=13.5 Hz, 1H, ½ CH₂), 1.09 (d, J=6.6 Hz, 6H, 2×valine CH₃). Purity: >95% (by NMR and HPLC).

LCMS: m/z=515.15; consistent for protonated parent ion.

### Example 3

### Synthesis of Oxycodone-[Glutaryl-(S)-Valine] Enol Ester and Oxycodone-[Glutaryl-(S)-Leucine] Enol Esters

### 1. Oxycodone-[glutaryl-(S)-valine] enol ester trifluoroacetate

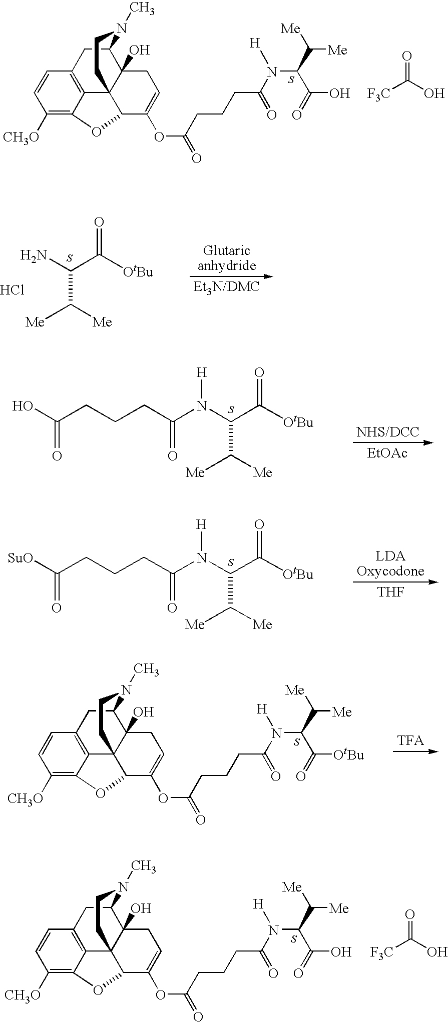

To (S)-valine tert-butyl ester hydrochloride (5.0 g, 23.8 mmol) and glutaric anhydride (2.99 g, 26.2 mmol) in dry dichloromethane (100 mL) was added triethylamine (7.6 mL, 54.7 mmol) dropwise and the resulting solution was stirred at room temperature for 3 hours. The solution was then washed with 5% aqueous citric acid (100 mL), saturated brine (100 mL), dried (MgSO₄) and concentrated to give glutaryl-[(S)-valine tert-butyl ester] (6.25 g, 91%), as a colourless oil.

To glutaryl-[(S)-valine tert-butyl ester] (6.25 g, 21.7 mmol) and N-hydroxysuccinimide (2.75 g, 23.9 mmol) in dry ethyl acetate (140 mL) was added N,N′-dicyclohexylcarbodi-imide (4.70 g, 22.8 mmol) and the mixture was stirred at room temperature overnight. The resulting suspension was filtered through Celite and the filtrate was washed with saturated aqueous sodium bicarbonate (140 mL), water (140 mL) and saturated brine (140 mL), dried (MgSO₄) and concentrated to give glutaryl-[(S)-valine-tert-butyl-ester] N-hydroxysuccinimide ester (7.30 g, 88%), as a pale yellow oil.

To a solution of oxycodone free base (4.00 g, 12.7 mmol) in dry THF (120 mL) at 0° C. was added lithium di-isopropylamide (7.7 mL of a 1.8 M solution in THF-heptane-ethylbenzene, 13.9 mmol) dropwise with stirring and the solution was then stirred for 30 minutes. A solution of glutaryl-[(S)-valine-tert-butyl-ester] N-hydroxysuccinimide ester (7.30 g, 19.0 mmol) in dry THF (230 mL) was added by cannula whilst maintaining the temperature at 0° C. The mixture was stirred overnight with warming to room temperature. The resulting suspension was filtered through Celite and the filtrate was concentrated to give the crude product as a yellow oil which was subjected to two rounds of purification on a Biotage Isolera automated chromatography system. The purification was carried out firstly under normal phase conditions (elution with a gradient of methanol:dichloromethane) and then under reversed phase conditions (C₁₈, elution with a gradient of 0→100% 0.1% aqueous TFA:acetonitrile) to give oxycodone-[glutaryl-(S)-valine tert-butyl ester] enol ester trifluoroacetate (1.90 g, 26%), as a white solid.

Oxycodone-[glutaryl-(S)-valine tert-butyl ester] enol ester trifluoroacetate (0.95 g, 16.2 mmol) was dissolved in trifluoroacetic acid (20 mL) and the mixture was stirred at room temperature for 1 hour. The mixture was concentrated and residual trifluoroacetic acid was removed azeotropically with chloroform (5×15 mL). The resulting solid was purified on a Biotage Isolera automated chromatography system under reversed phase conditions (C₁₈, elution with a gradient of 0→100% 0.1% aqueous TFA:acetonitrile) to afford oxycodone-[glutaryl-(S)-valine] enol ester trifluoroacetate (497 mg, 48%), as a white glassy solid.

### 2. Oxycodone-[glutaryl-(S)-leucine] enol ester Trifluoroacetate

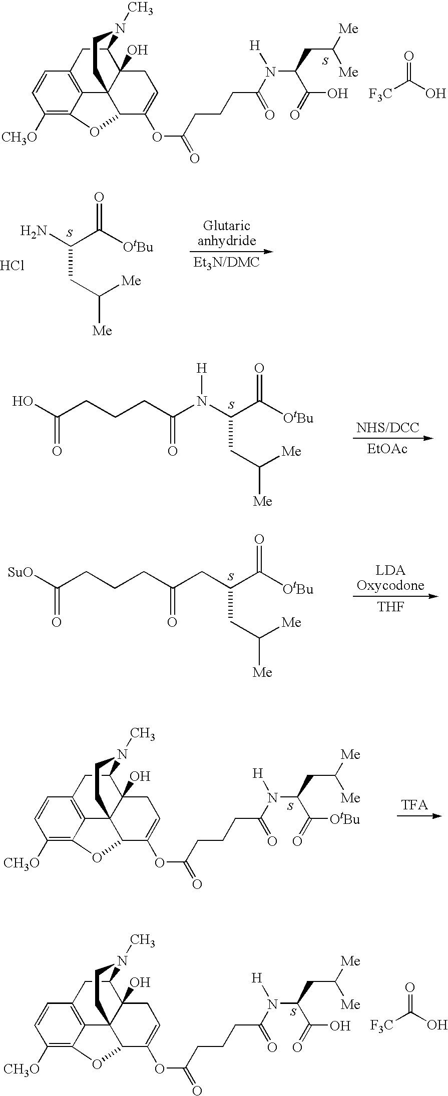

To (S)-leucine tert-butyl ester hydrochloride (5.00 g, 22.3 mmol) and glutaric anhydride (2.80 g, 24.5 mmol) in dry dichloromethane (125 mL) was added triethylamine (7.2 mL, 51.3 mmol) dropwise and the solution was then stirred at room temperature overnight. The resulting solution was washed with 5% aqueous citric acid (125 mL), water (125 mL) and saturated brine (125 mL), dried (MgSO₄) and concentrated to give glutaryl-[(S)-leucine tert-butyl ester] (6.65 g, 99%), as a colourless oil.

To glutaryl-[(S)-leucine tert-butyl ester] (6.65 g, 22.1 mmol) and N-hydroxysuccinimide (2.80 g, 24.3 mmol) in dry ethyl acetate (150 mL) was added N,N′-dicyclohexylcarbodi-imide (4.79 g, 23.2 mmol) and the mixture was stirred at room temperature overnight. The resulting suspension was filtered through Celite and the filtrate washed with saturated sodium bicarbonate (150 mL), water (150 mL) and saturated brine (150 mL), dried (MgSO₄) and concentrated to give glutaryl-[(S)-leucine-tert-butyl-ester] N-hydroxysuccinimide ester (8.71 g, 99%)., as a pale-yellow oil.

To a solution of oxycodone free base (4.60 g, 14.6 mmol) in dry THF (150 mL) was added lithium di-isopropylamide (8.9 mL of a 1.8 M solution in THF-heptane-ethylbenzene, 16.1 mmol) dropwise with stirring and the solution was stirred for 30 minutes. A solution of glutaryl-[(S)-leucine-tert-butyl-ester] N-hydroxysuccinimide ester (8.71 g, 21.9 mmol) in dry THF (250 mL) was added by cannula whilst maintaining the temperature at 0° C. The mixture was stirred overnight with warming to room temperature. The resulting suspension was filtered through Celite and the filtrate was concentrated to give the crude product as a yellow oil which was subjected to two rounds of purification on a Biotage Isolera automated chromatography system, firstly under normal phase conditions (elution with a gradient of methanol:dichloromethane) followed by reversed phase conditions (C₁₈, elution with a gradient of 0→100% 0.1% aqueous TFA:acetonitrile) to afford oxycodone-[glutaryl-(S)-leucine tert-butyl ester] enol ester trifluoroacetate (2.64 g, 30%).

Oxycodone-[glutaryl-(S)-leucine tert-butyl ester] enol ester trifluoroacetate (1.32 g, 2.20 mmol) was dissolved in trifluoroacetic acid (30 mL) and the mixture was stirred at room temperature for 1 hour. The mixture was concentrated and residual trifluoroacetic acid was removed azeotropically with chloroform (5×30 mL). The resulting solid was purified on a Biotage Isolera automated chromatography system under reversed phase conditions (C₁₈, elution with a gradient of 0→100% 0.1% aqueous TFA:acetonitrile) to give oxycodone-[glutaryl-(S)-leucine] enol ester trifluoroacetate (519 mg, 36%).

### Example 4

### Synthesis of Codeine-[Succinyl-(S)-Valine] Trifluoroacetate

Succinyl-(S)-valine-tert-butyl ester was synthesized according to a literature method (Stupp et al. (2003). J. Am. Chem. Soc. 125, 12680-12681) by reacting (S)-valine tert-butyl ester hydrochloride with succinic anhydride in dichloromethane in the presence of triethylamine. After an aqueous work-up, the product was isolated in good yield and purity by crystallization from diethyl ether petrol, as a fluffy white powder.

Codeine was then coupled with succinyl-(S)-valine-tert-butyl ester. The reaction was mediated by dicyclohexylcarbodi-imide (DCC) in dichloromethane and catalyzed by N,N-dimethylaminopyridine (DMAP). The reaction proceeded to give an 97% yield of the half-ester in good purity after chromatography. Trifluoroacetic acid (TFA) deprotection of the valine carboxyl group followed by crystallization by trituration with diethyl ether-tetrahydrofuran, afforded codeine-[succinyl-(S)-valine]ester trifluoroacetate in quantitative yield, as a white powder. These steps are shown in Scheme 4, below.

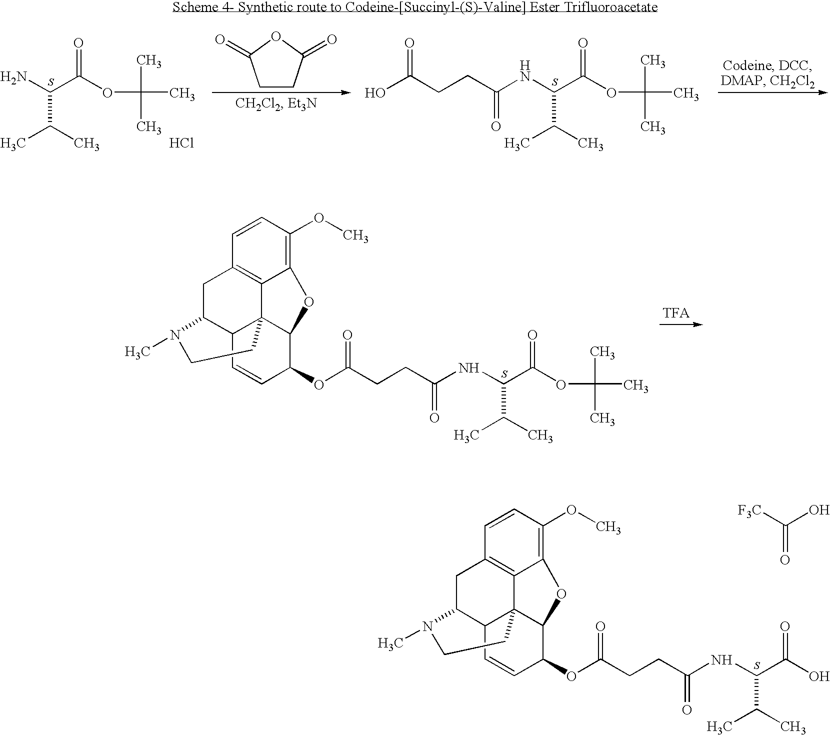

### Experimental Details

Et₃N (7.3 mL, 5.3 g, 52.5 mmol) was added dropwise to a suspension of (S)-valine tert-butyl ester hydrochloride (5.0 g, 23.9 mmol) and succinic anhydride (2.50 g, 25.0 mmol) in anhydrous CH₂Cl₂ (125 mL) under N₂. The resulting solution was stirred for 3 hours. Further CH₂Cl₂ (250 mL) was added and the solution was washed with 5% aqueous citric acid (2×250 mL) and brine (250 mL), dried (MgSO₄), and concentrated. The resulting oil was crystallized from diethyl ether petrol and the product collected by filtration. The product was then washed with-petrol and dried under vacuum to afford succinyl-(S)-valine-O-tert-butyl ester (6.17 g, 94%), as a fluffy white solid.

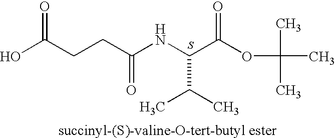

¹H NMR (CDCl₃, 300 MHz): δ 6.38 (d, J=9.0 Hz, 1H, NH), 4.48 (dd, J=9.0, 6.0 Hz, 1H, valine α-CH), 2.75 (m, 2H, succinyl CH₂), 2.62 (m, 2H, succinyl CH₂), 2.16 (m, 1H, valine β-CH), 1.49 (s, 9H, tert-butyl), 0.93 (m, 6H, 2×valine CH₃).

Solid DCC (1.96 g, 9.50 mmol) was added portionwise to a solution of codeine free base (1.98 g, 6.62 mmol), [succinyl-(S)-valine]-O-tert-butyl ester (2.53 g, 9.27 mmol) and DMAP (28 mg, 0.23 mmol) under N₂ in anhydrous CH₂Cl₂ (42 mL). The solution was stirred overnight, filtered through celite with CH₂Cl₂ and rinsed with EtOAc to remove dicyclohexylurea. The filtrate was then concentrated. Medium-pressure chromatography on silica, eluting with a gradient of 2→10% methanol in dichloromethane containing 0.1% triethylamine, afforded tert-butyl-protected codeine-[succinyl-(S)-valine]ester as a foam, (3.50 g, 95%). R_{f} 0.28 (9:1 dichloromethane-methanol plus trace Et₃N).

This material was stirred in trifluoroacetic acid (76 mL) for 15 minutes, then concentrated and azeotroped three times with CHCl₃. The residue was crystallized by trituration with diethyl ether-THF, and the resulting product was collected by filtration, washed with diethyl ether and dried under vacuum at 50° C. to afford codeine-[succinyl-(S)-valine]ester trifluoroacetate (3.24 g, 84%), as a white powder.

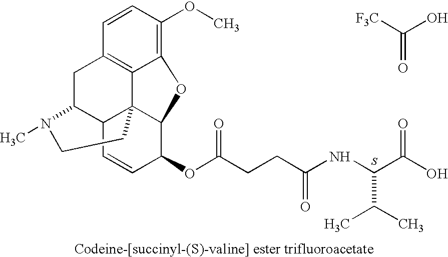

¹H NMR (DMSO-d₆, 300 MHz): δ 8.07 (d, J=8.7 Hz, 1H, amide NH), 6.78 (d, J=8.4 Hz, 1H, ArH), 6.65 (d, J=8.4 Hz, 1H, ArH), 5.66 (d, J=10.5 Hz, 1H, alkene H), 5.47 (d, J=10.5 Hz, 1H, alkene H), 5.18 (broad, 1H, CH—O.CO), 5.11 (d, J=6.9 Hz, 1H, CH—O—Ar), 4.15 (m, 2H, valine α-CH+CHN), 3.76 (s, 3H, ArOCH₃), 3.4-3.0 (m, 2H, CH₂N), 2.89 (s, 3H, CH₃N), ca. 2.8 (broad m, 2H, ArCH₂), 2.6-1.8 (m, 8H, codeine CH₂+codeine CH+2×succinyl CH₂+valine β CH), 0.87 (d, J=6.6 Hz, 6H, 2×valine CH₃).

LCMS (positive ionization): m/z=499.27; consistent for protonated parent ion.

### Example 5

### Synthesis of Dihydrocodeine-[Succinyl-(S)-Valine] Ester Trifluoroacetate

This synthetic route for dihydrocodeine-[succinyl-(S)-valine]ester trifluoroacetate is shown in Scheme 5.

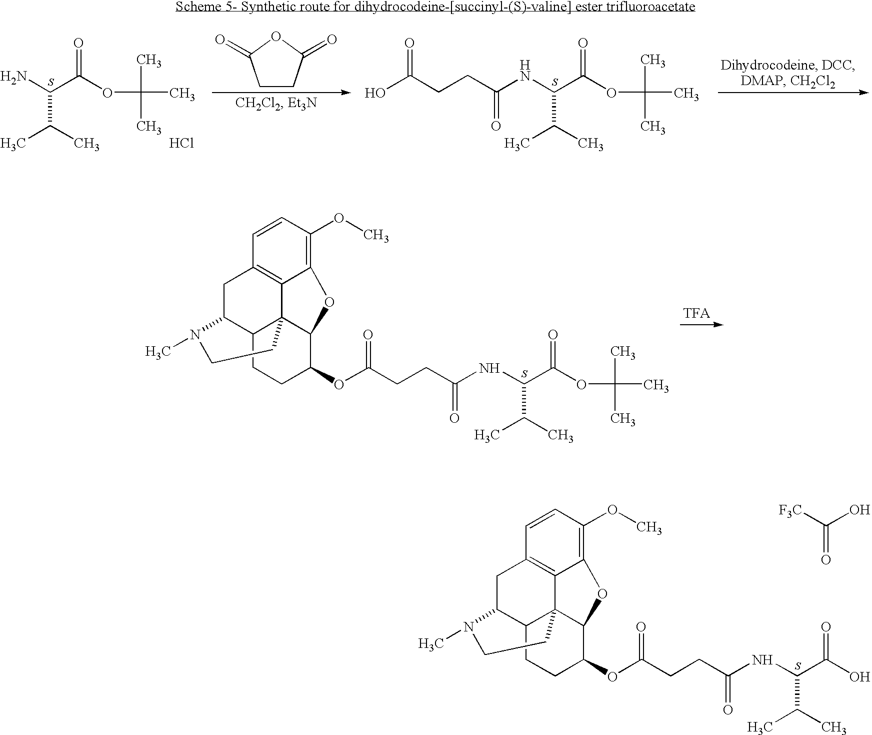

DCC-mediated coupling of dihydrocodeine with succinyl-(S)-valine-tert-butyl ester in dichloromethane catalyzed by 4-dimethylaminopyridine (DMAP) gave a 79% yield of the half-ester in good purity after chromatography.

Trifluoroacetic acid (TFA) deprotection removed the tert-butyl protecting group, and the product was concentrated to a foam which was triturated with diethyl ether alone to afford dihydrocodeine-[succinyl-(S)-valine]ester trifluoroacetate in good yield, as a white powder.

### Experimental Details

Solid DCC (3.61 g, 17.5 mmol) was added portionwise to a solution of dihydrocodeine free base (3.76 g, 12.5 mmol), [succinyl-(S)-valine]-O-tert-butyl ester (4.77 g, 17.5 mmol) and DMAP (125 mg, 0.25 mmol) under N₂ in anhydrous CH₂Cl₂ (100 mL). The solution was stirred overnight, filtered through celite with CH₂Cl₂ and rinsed with EtOAc to remove dicyclohexylurea. The filtrate was then concentrated. Medium-pressure chromatography on silica, eluting with a gradient of 2→12% methanol in dichloromethane containing 0.1% triethylamine, afforded tert-butyl-protected dihydrocodeine-[succinyl-(S)-valine]ester (2.34 g, 34%), as a foam. R_{f} 0.24 (9:1 dichloromethane-methanol plus trace Et₃N).

This material was stirred in trifluoroacetic acid (53 mL) for 15 minutes, then concentrated and azeotroped three times with CHCl₃. The residue was evaporated to a foam which was dissolved in ethanol (10 mL), and diethyl ether was added to induce precipitation. The white solid formed was collected by filtration, triturated with diethyl ether and dried under vacuum at 50° C. to afford the title compound as a white powder, (1.60 g, 62%).

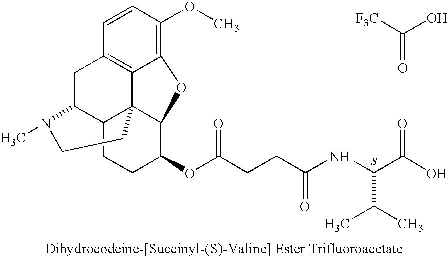

¹H NMR (DMSO-d₆, 300 MHz): δ 9.70 (s, 1H, NH⁺), 7.94 (d, J=8.4 Hz, 1H, amide NH), 6.84 (d, J=8.1 Hz, 1H, ArH), 6.74 (d, J=8.4 Hz, 1H, ArH), 5.25 (broad, 1H, CH—O.CO), 4.84 (d, J=6.0 Hz, 1H, CH—O—Ar), 4.08 (m, 1H, valine α-CH), 3.88 (m, 1H, CHN), 3.76 (s, 3H, ArOCH₃), ca. 3.5+3.21 (AB system, J=19.5 Hz, 2H, CH₂N), 2.85 (s, 3H, CH₃N), 2.6-1.3 (m, 10H, ArCH₂+codeine CH₂+codeine CH+2×succinyl CH₂+valine β CH), 0.85 (d, J=6.6 Hz, 6H, 2×valine CH₃).

LCMS (positive ionization): m/z=501.13; consistent for protonated parent ion.

### Example 6

### Synthesis of Oxymorphone-[Succinyl-(S)-Valine] Ester

Triethylamine (1.31 mL, 9.43 mmol) was added dropwise, with stirring, to a suspension of (S)-valine benzyl ester hydrochloride (1.0 g, 4.10 mmol) and succinic anhydride (0.46 g, 4.51 mmol) in anhydrous dichloromethane (30 mL). Stirring was continued for a further period of 3 hours. The resulting mixture was diluted with dichloromethane (100 mL) and washed with 5% aqueous citric acid (2×100 mL), followed by brine. The product was then dried (MgSO₄) and concentrated to give succinyl (S)-valine benzyl ester (1.22 g), as an oil.

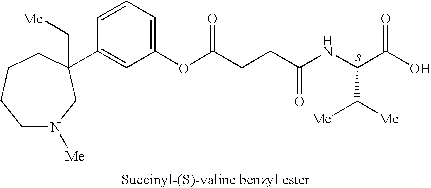

¹H NMR (300 MHz, DMSO-d₆) δ 12.07 (broad s, 1H, CO₂H), 8.19 (d, J=8.1 Hz, NH), 7.37 (m, 5H, 5×PhH), 5.15+5.09 (AB system, J=12.3 Hz, benzylic CH₂), 4.21 (dd, J=8.1, 6.6 Hz, 1H, valine α-CH), 2.45-2.40 (m, 4H, 2×succinyl CH₂), 2.03 (m, 1H, valine β-CH), 0.86 (d, J=3.9 Hz, 3H, valine CH₃), 0.84 (d, J=3.9 Hz, 3H, valine CH₃).

Dicyclohexylcarbodi-imide (0.76 g, 3.70 mmol) was added to solution of succinyl-(S)-valine benzyl ester (1.07 g, 3.50 mmol) and oxymorphone free base (0.80 g, 2.66 mmol) in anhydrous dichloromethane (20 mL) under nitrogen. The mixture was stirred overnight at room temperature, filtered through celite and concentrated to an oil. The oil was purified by silica chromatography eluting with a gradient of 2→10% methanol in dichloromethane containing 0.1% triethylamine, to give the benzyl ester of oxymorphone-[succinyl-(S)-valine]ester (1.43 g), as a white foam.

A solution of oxymorphone-[succinyl-(S)-valine] benzyl ester (410 mg, 0.69 mmol) and acetic acid (60 μL, 63 mg, 1.04 mmol) in ethanol (15 mL) was added to a slurry of 10% Pd/C (250 mg) in ethanol (5 mL, the ethanol having been added to Pd/C under N₂). The flask was evacuated, an atmosphere of hydrogen was added via a balloon, and the suspension was stirred overnight. After this time, the catalyst was removed by filtration through celite, and the solvent was evaporated. The resulting residue was triturated with ether, collected by suction filtration, and dried under vacuum at 70° C. for 7 hr to give the desired oxymorphone [succinyl-(S)-valine]ester (260 mg, 75%), as a white solid.

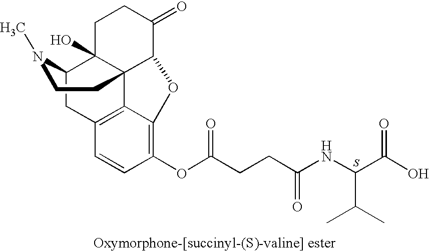

¹H NMR (DMSO-d₆): 8.09 (d, J=8.4 Hz, 1H, amide NH), 6.82 (d, J=8.1 Hz, 1H, ArH), 6.74 (d, J=8.1 Hz, 1H, ArH), 5.90 (s, 1H, CH—O—Ar), 4.15 (dd, J=8.4, 5.7 Hz, 1H, valine α-CH), 3.30 (obscured m, 1H, CHN), 3.15 (d, J=18.9 Hz, 1H, ½×CH₂N), 2.89 (dd, J=11.4, 5.7 Hz, 2H, benzylic CH₂), 2.75 (dd, J=11.4, 5.7 Hz, 2H, succinyl CH₂), 2.6-2.4 (m, 4H, succinyl CH₂+½×CH₂N+½×CH₂), 2.34 (s, 3H, CH₃N), 2.15-1.95 (m, 3H, valine β CH+CH₂), 1.75 (m, 1H, ½×CH₂), 1.45 (m, 1H, ½×CH₂), 1.35 (m, 1H, ½×CH₂), 0.87 (d, J=6.6 Hz, 6H, 2×valine CH₃).

LCMS: m/z=500.87, consistent for protonated parent ion.

### Example 7

### Synthesis of Hydrocodone-[Succinyl-(S)-Valine] Enol Ester Trifluoroacetate

The activated ester N-hydroxysuccinimidyl-succinyl-(S)-valine tert-butyl ester was prepared by reacting (S)-valine tert-butyl ester hydrochloride with succinic anhydride, followed by activation with N-hydroxysuccinimide (Scheme 6).

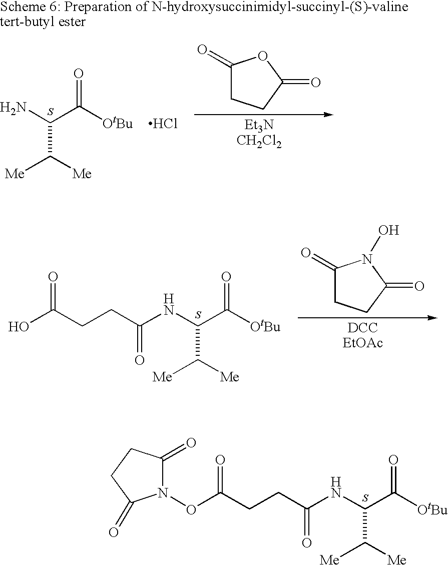

A solution of hydrocodone enolate was prepared by treating a solution of hydrocodone in anhydrous tetrahydrofuran with lithium di-isopropylamide (LDA). A solution of N-hydroxysuccinimidyl-succinyl-(S)-valine tert-butyl ester in tetrahydrofuran was added to the enolate solution. Purification by column chromatography gave hydrocodone-[succinyl-(S)-valine-tert-butyl ester] enol ester as a foam in good yield. The tert-butyl ester was removed by treatment with trifluoroacetic acid to give hydrocodone-[succinyl-(S)-valine] enol ester trifluoroacetate as a tan gum in good yield (Scheme 7).

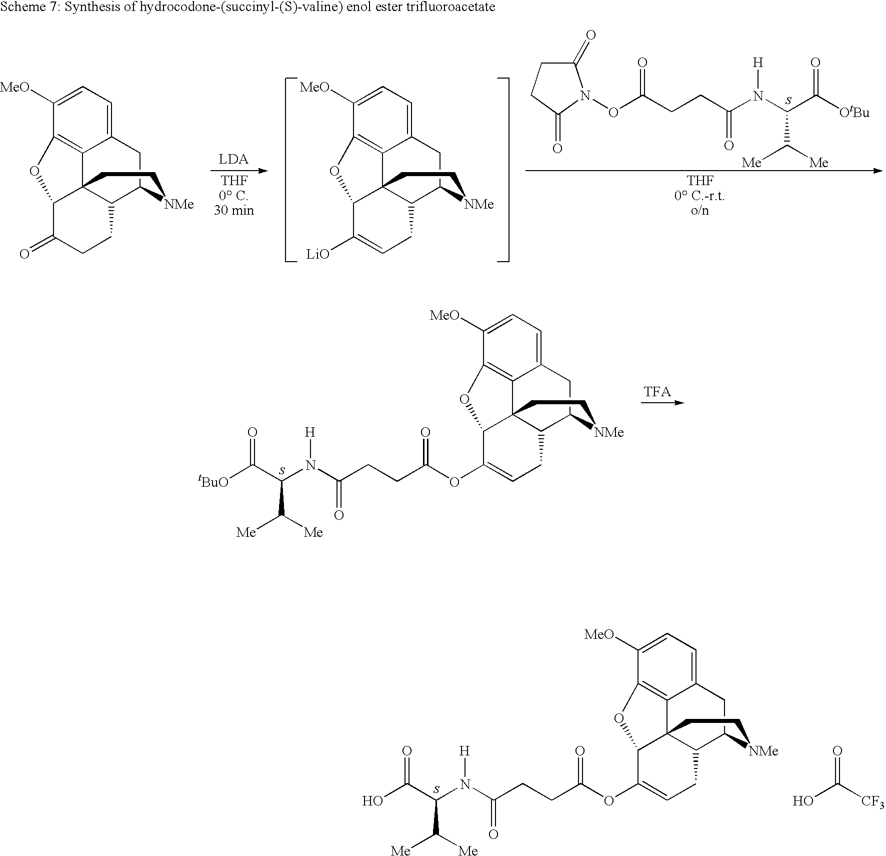

### Example 8

### Synthesis of Meptazinol-[Succinyl-(S)-Valine] Ester

The synthesis of meptazinol-[succinyl-(S)-valine]ester was achieved in three distinct steps as shown in Scheme 8. (S)-Valine benzyl ester was first reacted with succinic anhydride to give succinyl-(S)-valine benzyl ester. This was then coupled with meptazinol free base mediated by dicyclohexylacarbodi-imide (DCC) to yield meptazinol-[succinyl-(S)-valine] benzyl ester after purification by chromatography. Subsequent deprotection by hydrogenolysis in the presence of a palladium on carbon catalyst resulted in the desired formation of meptazinol-[succinyl-(S)-valine]ester as a white solid.

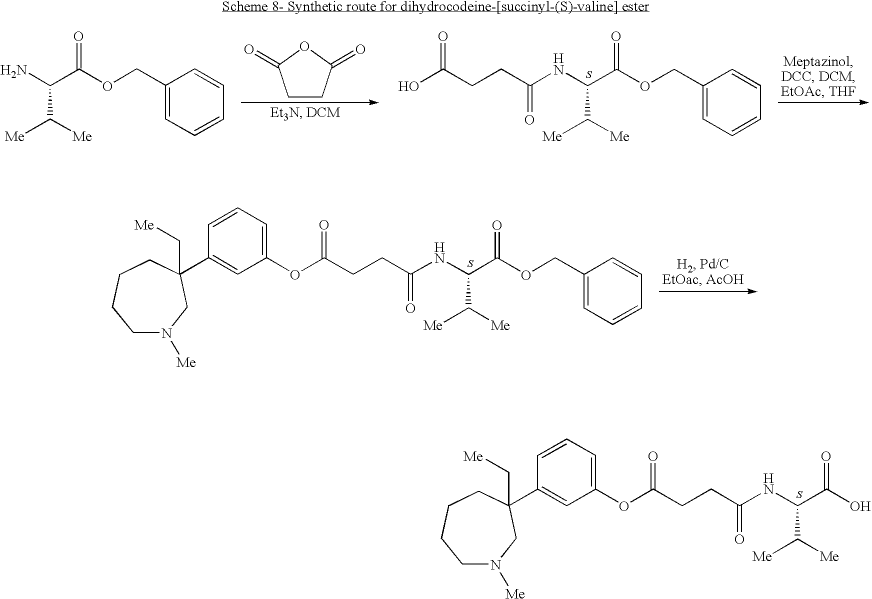

### Experimental Details

Triethylamine (1.31 mL, 9.43 mmol) was added dropwise, with stirring, to a suspension of (S)-valine benzyl ester hydrochloride (1.0 g, 4.10 mmol) and succinic anhydride (0.46 g, 4.51 mmol) in anhydrous dichloromethane (30 mL). Stirring was continued for a further period of 3 hours. The resulting mixture was diluted with dichloromethane (100 mL) and washed with 5% aqueous citric acid (2×100 mL), followed by brine. The product was then dried (MgSO₄) and concentrated to give succinyl (S)-valine benzyl ester (1.22 g), as an oil.

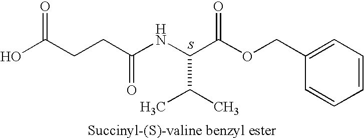

¹H NMR (300 MHz, DMSO-d₆) δ 12.07 (broad s, 1H, CO₂H), 8.19 (d, J=8.1 Hz, NH), 7.37 (m, 5H, 5×PhH), 5.15+5.09 (AB system, J=12.3 Hz, benzylic CH₂), 4.21 (dd, J=8.1, 6.6 Hz, 1H, valine α-CH), 2.45-2.40 (m, 4H, 2×succinyl CH₂), 2.03 (m, 1H, valine β-CH), 0.86 (d, J=3.9 Hz, 3H, valine CH₃), 0.84 (d, J=3.9 Hz, 3H, valine CH₃).

Dicyclodicarbodi-imide (0.98 g, 4.74 mmol) was added to solution of succinyl-(S)-valine benzyl ester (1.20 g, 3.91 mmol), meptazinol free base (1.10 g, 4.74 mmol) in ethyl acetate (10 mL), anhydrous tetrahydrofuran (6 mL) and anhydrous dichloromethane (6 mL), cooled in an ice-bath under nitrogen. The mixture was stirred overnight at room temperature, filtered through Celite and concentrated to an oil. The oil was purified by silica chromatography, eluting with a mixture of dichloromethane:methanol (20:1) containing 0.1% triethylamine. This afforded meptazinol [succinyl-(S)-valine] benzyl ester (1.61 g), as a colourless oil.

The purified material (0.4 g, 0.77 mmol) and acetic acid (44 μL, 0.77 mmol) were added to ethyl acetate (15 mL) and stirred with 10% Pd/C (0.20 g) under a hydrogen atmosphere at room temperature for 4 hours. After this time, the catalyst was filtered and the solvent was then evaporated. The resulting residue was triturated with petrol ether to give the desired meptazinol-[succinyl-(S)-valine]ester (0.27 g, 81%), as a white solid.

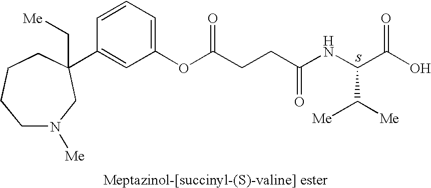

¹H NMR (300 MHz, DMSO-d₆) δ 8.10 (d, J=8.6 Hz, 1H, NH), 7.32 (t, J=7.9 Hz, 1H, ArH), 7.20 (d, J=7.9 Hz, 1H, ArH), 7.02 (s, 1H, ArH), 6.91 (d, J=7.8 Hz, 1H, ArH), 4.18 (m, 1H, β-CH), 2.76-1.99 (m, 9H, 4×CH₂+β-CH), 2.32 (s, 3H, NCH₃), 1.61-1.24 (m, 8H, 4×CH₂), 0.88 (d, J=6.7 Hz, 6H, 2×isopropyl CH₃), 0.51 (t, J=7.3 Hz, 3H, CH₃).

### Example 9

### Synthesis of Des-methyl meptazinol hydrobromide

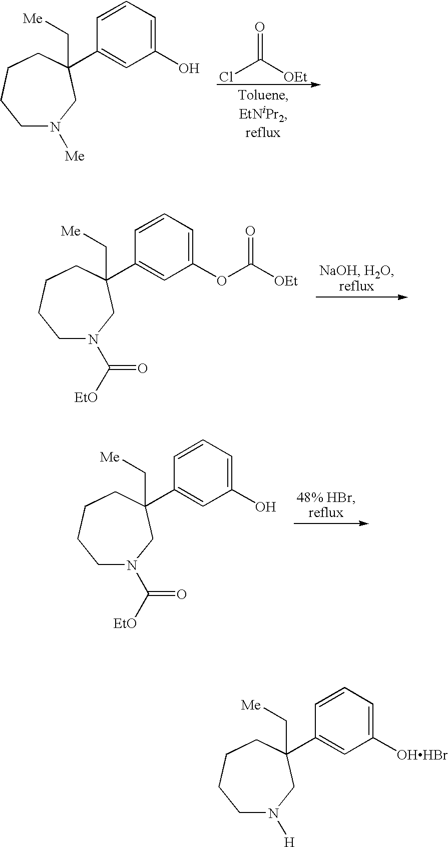

### Example 10

### Synthesis of ethyl-hydroxylated meptazinol

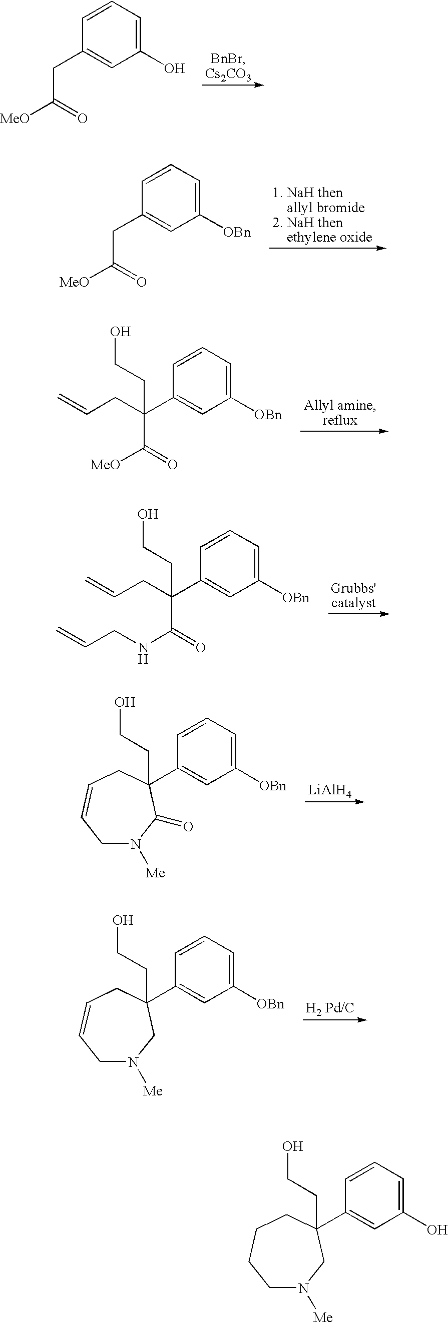

### Example 11

### Synthesis of ethyl-carboxylated meptazinol

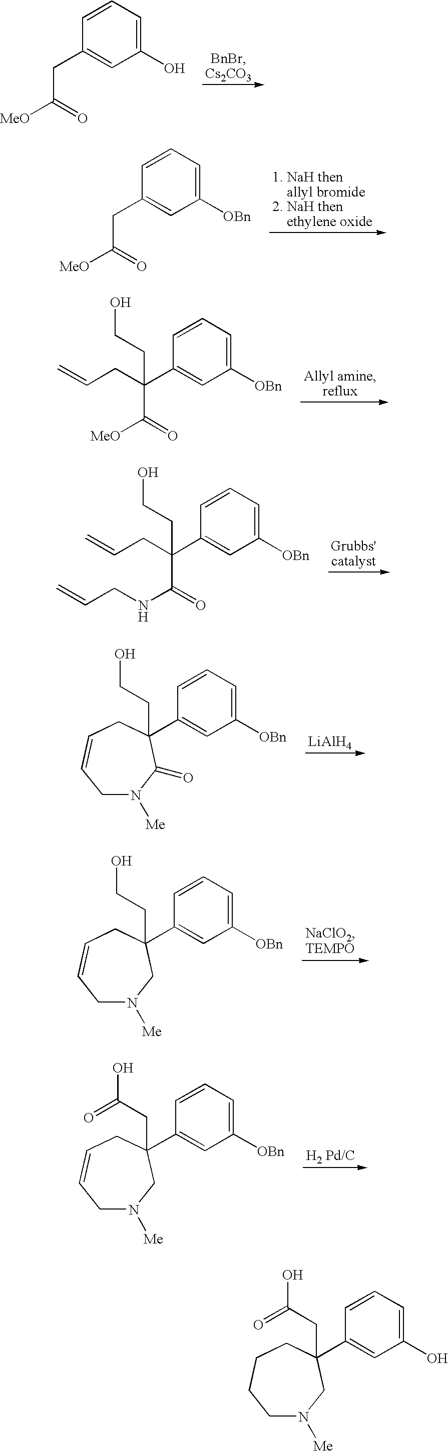

### Examples 12

### Synthesis Meptazinol [Phthalyl-(S)-Valine] Ester Trifluoroacetate

The synthesis of meptazinol [phthalyl-(S)-valine]ester trifluoroacetate was achieved using the route set out below:

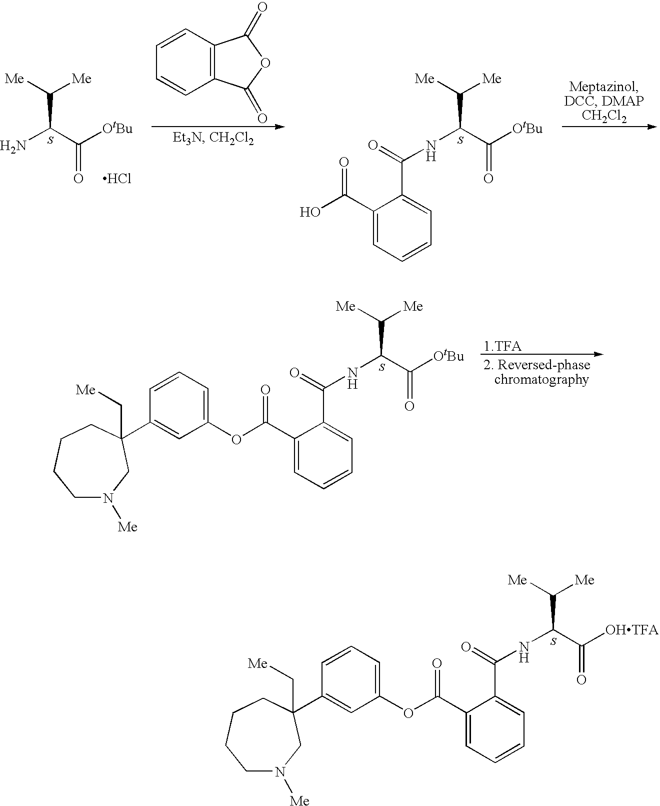

### Synthetic route for meptazinol [phthalyl-(S)-valine]ester trifluoroacetate

The reaction of (S)-valine tert-butyl ester hydrochloride with phthalic anhydride in dichloromethane in the presence of triethylamine afforded after an aqueous work-up, the amino-acid-linker conjugate in high yield and good purity by ¹H NMR (>95%).

The linker was coupled to meptazinol using N,N′-dicyclohexylcarbodi-imide (DCC) in dichloromethane in the presence of N,N-dimethylaminopyridine (DMAP) catalyst to afford the corresponding tert-butyl protected meptazinol [phthalyl-(S)-valine]ester, which was purified by column chromatography. Finally, removal of the tert-butyl ester in neat trifluoroacetic acid (TFA), followed by reversed-phase chromatographic purification, gave the trifluoroacetate salt of the target compound in good purity (>95%).

Details of Preparation of [phthalyl-(S)-valine] tert-butyl ester

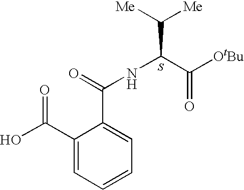

To a solution of (S)-valine tert-butyl ester hydrochloride (1.00 g, 4.76 mmol) and phthalic anhydride (0.78 g, 5.24 mmol) in dichloromethane (30 mL) was added triethylamine (1.53 mL, 1.11 g, 11.0 mmol) and the reaction mixture was stirred for 3 hours. The solution was then diluted with dichloromethane (50 mL), washed with 10% citric acid (2×50 mL), brine (50 mL), dried (MgSO₄) and concentrated to give [phthalyl-(S)-valine] tert-butyl ester (1.43 g, 93%), as an oil.

Details of Preparation of meptazinol [phthalyl-(S)-valine] trifluoroacetate ester

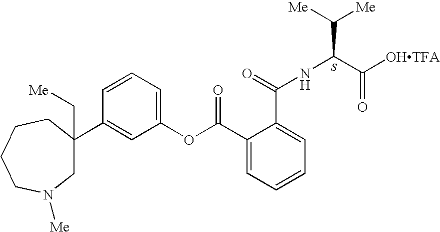

To a solution of [phthalyl-(S)-valine] tert-butyl ester (1.53 g, 4.76 mmol) and meptazinol free base (0.85 g, 3.66 mmol) in dichloromethane (35 mL) was added N,N′-dicyclohexylcarbodi-imide (1.06 g, 5.13 mmol) and N,N-dimethylaminopyridine (9 mg, 0.07 mmol) and the reaction was stirred overnight. The resulting suspension was filtered through Celite and concentrated. The residue was purified by medium-pressure chromatography on silica eluting with a gradient of 2→4% (9; 1 v/v methanol-NH₄OH) in dichloromethane to afford meptazinol [phthalyl-(S)-valine] tert-butyl ester (1.40 g, 71%), as a clear oil. R_{f} 0.50 (10% methanol-90% dichloromethane).

A portion of the purified material (0.57 g, 1.1 mmol) was dissolved in trifluoroacetic acid (12 mL) and stirred at room temperature for 1 hour. The mixture was evaporated and the residual trifluoroacetic acid was removed azeotropically with chloroform (5×25 mL). The crude material was purified using a Biotage Isolera automated chromatography system under reversed-phase conditions (C₁₈ column, 0→100% MeCN in 0.1% aqueous TFA) to give, after freeze-drying, meptazinol [phthalyl-(S)-valine]ester trifluoroacetate (361 mg, 55%), as a white solid.

¹H NMR (300 MHz, DMSO-d₆): δ 9.11+8.38 (2×bs, 1H, NH⁺) 8.57-8.54 (m, 1H, NH), 7.66 (d, J=7.4 Hz, 1H, ArH), 7.51-7.38 (m, 3H, 3×ArH), 7.30-7.21 (m, 1H, ArH), 7.12-6.89 (m, 3H, 3×ArH), 4.08 (t, J=6.6 Hz, 1H, α-CH), 3.76-3.66 (m, 0.5H, 0.25×NCH₂), 3.41-3.16 (m, 1.5H, 0.75×NCH₂), 3.04-2.87 (m, 2H, NCH₂), 2.71-2.61 (m, 3H, NCH₃), 2.21-2.13 (m, 0.5H, 0.25×CH₂), 2.05-1.83 (m, 1.5H, 0.75×CH₂), 1.78-1.39 (m, 5H, 2×CH₂+β-CH), 1.39-1.34 (m, 2H, CH₂), 0.72-0.60 (m, 6H, 2×CH₃), 0.35-0.23 (m, 3H, CH₃).

LCMS (Positive mode): Single peak m/z=480.88, consistent for protonated parent ion (MH⁺)

### Examples 13

### Synthesis Meptazinol [Phthalyl-(S)-phenylalanine] Ester Trifluoroacetate

The synthesis of meptazinol [phthalyl-(S)-phenylalanine]ester trifluoroacetate was achieved using the route set out below:

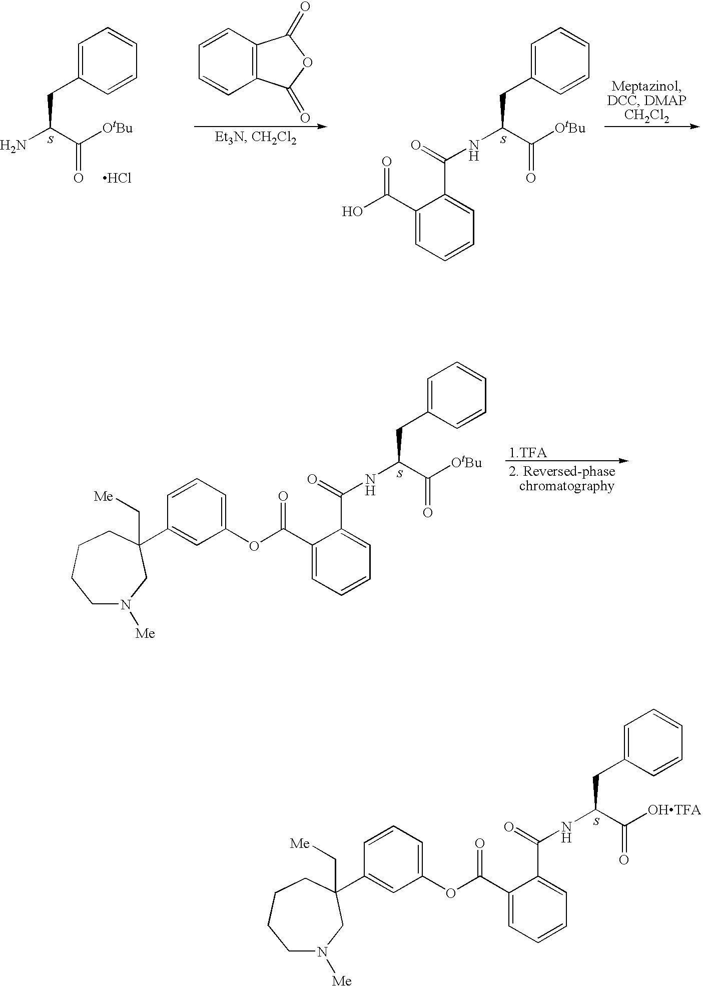

The reaction of (S)-phenylalanine tert-butyl ester hydrochloride with phthalic anhydride in dichloromethane in the presence of triethylamine afforded, after an aqueous work-up, the required amino-acid-linker conjugate in high yield and good purity by ¹H NMR (>95%).

The linker was coupled to meptazinol using N,N′-dicyclohexylcarbodi-imide (DCC) in dichloromethane in the presence of N,N-dimethylaminopyridine (DMAP) to afford the corresponding tert-butyl protected meptazinol [phthalyl-(S)-phenylalanine]ester, which was purified by column chromatography. Finally, removal of the tert-butyl ester using neat trifluoroacetic acid (TFA), followed by reversed-phase chromatographic purification, gave the trifluoroacetate salt of the target compound in good purity (>95%).

Details of Preparation of [phthalyl-(S)-phenylalanine] tert-butyl ester

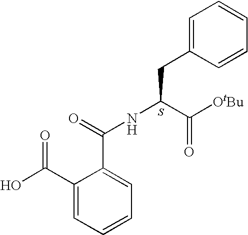

To a solution of (S)-phenylalanine tert-butyl ester hydrochloride (1.00 g, 3.88 mmol) and phthalic anhydride (0.63 g, 4.26 mmol) in dichloromethane (30 mL) was added triethylamine (1.24 mL, 0.90 g, 8.92 mmol) and the reaction mixture was stirred for 3 hours. The solution was diluted with dichloromethane (50 mL), washed with 10% citric acid (2×50 mL), brine (50 mL), dried (MgSO₄) and concentrated to give [phthalyl-(S)-phenylalanine] tert-butyl ester (1.43 g, 100%), as an oil.

Details of Preparation of meptazinol [phthalyl-(S)-phenylalanine] ester trifluoroacetate

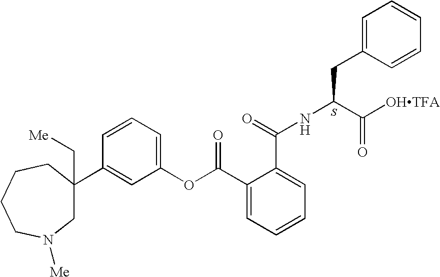

To a solution of [phthalyl-(S)-phenylalanine] tert-butyl ester (1.43 g, 3.88 mmol) and meptazinol free base (0.75 g, 3.23 mmol) in dichloromethane (35 mL) was added N,N′-dicyclohexylcarbodiimide (0.93 g, 4.52 mmol) and N,N-dimethylaminopyridine (8 mg, 0.06 mmol) and the reaction was stirred overnight. The resulting suspension was filtered through Celite and concentrated. The residue was purified by medium-pressure chromatography on silica eluting with a gradient 2→4% (9:1 v/v methanol-NH₄OH) in dichloromethane to afford meptazinol [phthalyl-(S)-phenylalanine] tert-butyl ester (1.15 g, 61%), as a clear oil. R_{f} 0.50 (10% methanol-90% dichloromethane).

A portion of the purified material (0.55 g, 0.93 mmol) was dissolved in trifluoroacetic acid (11 mL) and stirred at room temperature for 45 minutes. The mixture was evaporated and the residual trifluoroacetic acid was removed azeotropically with chloroform (5×25 mL). The crude material was purified using a Biotage Isolera automated chromatography system under reversed-phase conditions (C₁₈ column, 0→100% MeCN in 0.1% aqueous TFA) to give, after freeze-drying, meptazinol [phthalyl-(S)-phenylalanine]ester trifluoroacetate (348 mg, 58%), as a white solid.

¹H NMR (300 MHz, DMSO-d₆): δ 9.26+8.59 (2×bs, 1H, NH⁺) 9.01 (d, J=8.0 Hz, 1H, NH), 7.85-7.81 (m, 1H, ArH), 7.73-7.62 (m, 2H, 2×ArH), 7.60-7.44 (m, 2H, 2×ArH), 7.35-7.09 (m, 8H, 8×ArH), 4.69-4.60 (m, 1H, α-CH), 3.98-3.91 (m, 0.5H, 0.25×NCH₂), 3.64-3.57 (m, 0.5H, 0.25×NCH₂), 3.53-3.39 (m, 1.5H, 0.75×NCH₂), 3.24-3.10 (m 2.5H, 0.75×NCH₂=0.5×CH₂Ph), 3.06-2.97 (m, 1H, 0.5×CH₂), 2.92-2.85 (m, 3H, NCH₃), 2.47-2.38 (m, 0.5H, 0.25×CH₂), 2.28-2.14 (m, 0.5H, 0.25×CH₂), 2.01-1.61 (m, 5H, 2.5×CH₂), 1.58-1.44 (m, 2H, CH₂), 0.56-0.41 (m, 3H, CH₃).

LCMS (Positive mode): Single peak m/z=529.30, consistent for protonated parent ion (MH⁺).

### Example 14

### Synthesis of Buprenorphine-[Succinyl-(S)-Valine] Ester

Buprenorphine-[succinyl-(S)-valine]ester was prepared starting from succinyl-(S)-valine benzyl ester (made by treating (S)-valine benzyl ester hydrochloride with succinic anhydride in the presence of triethylamine in dichloromethane) using DCC as coupling agent followed by catalytic hydrogenolysis of the benzyl group.

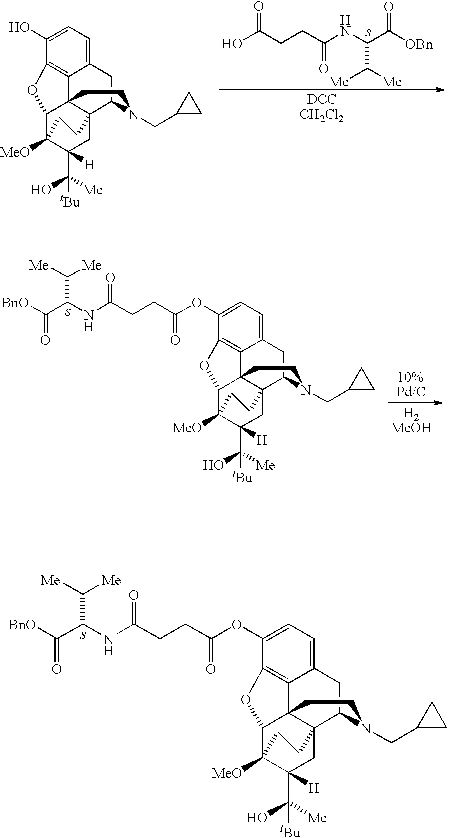

### Synthesis of Buprenorphine-[succinyl-(S)-valine]ester

Details of Preparation of buprenorphine-[succinyl-(S)-valine] ester

To a stirred solution of succinyl-(S)-valine benzyl ester (0.72 g, 2.35 mmol) and buprenorphine free base (0.84 g, 1.80 mmol) in anhydrous dichloromethane (10 mL) was added dicyclohexylcarbodiimide (0.52 g, 2.53 mmol) and the resulting suspension was stirred at room temperature overnight. The reaction mixture was filtered through Celite and concentrated. Purification by medium pressure column chromatography (eluent 2% methanol in dichloromethane; product R_{f} 0.78 in 10% methanol in dichloromethane) gave buprenorphine-[succinyl-(S)-valine benzyl ester]-ester as a white foam (0.75 g, 55%).

10% Palladium on carbon (150 mg) was cautiously wetted with ethyl acetate (2 mL) under an atmosphere of nitrogen. A solution of buprenorphine-[succinyl-(S)-valine benzyl ester]-ester (746 mg, 0.99 mmol) in anhydrous methanol (20 mL) was added to the reaction flask, which was evacuated. An atmosphere of hydrogen was introduced via a balloon and the reaction was stirred overnight. The reaction mixture was filtered through Celite and concentrated to a white solid. Purification by medium pressure column chromatography (eluent 10% methanol in dichloromethane; product R_{f} 0.28 in 10% methanol in dichloromethane) gave buprenorphine-[succinyl-(S)-valine]ester (360 mg, 55%), as a white solid.

¹H-NMR (DMSO-d₆, 300 MHz): 12.54 (br s, 1H, CO₂H), 7.97 (d, J=8.6 Hz, 1H, NH), 6.71 (d, J=8.1 Hz, 1H, ArH), 6.54 (d, J=8.1 Hz, 1H, ArH), 5.41 (s, 1H, CHO), 4.32 (s, 1H, CHN), 4.08-4.03 (m, 1H, valine α-CH), 3.26 (s, 3H, OCH₃), 2.91-2.85 (m, 2H, CH₂), 2.72-2.62 (m, 3H, CH₂+valine β-CH), 2.53-2.46 (m, 2H, CH₂), 2.35-2.04 (m, 4H, 2×CH₂), 1.98-1.77 (m, 4H, 2×CH₂), 1.69-1.49 (m, 3H, CH₂+CH), 1.28-1.16 (m, 4H, CH+CH₃), 1.07-0.95 (m, 2H, CH₂), 0.86 (s, 9H, tert-butyl), 0.77 (d, J=6.8 Hz, 6H, 2×valine CH₃), 0.45-0.29 (m, 2H, cyclopropyl CH₂), 0.06-−0.05 (m, 2H, cyclopropyl CH₂).

HPLC indicated the purity to be >99%.

LCMS showed m/z at 666.96 (consistent with protonated ion MH⁺)

### Example 15

### Synthesis of Buprenorphine-[Glutaryl-(S)-Valine] Ester

This was made in analogous manner to the corresponding succinyl valine ester.

The glutaryl-(S)-valine benzyl ester linker was coupled to buprenorphine using DCC in dichloromethane. After purification by flash chromatography, buprenorphine-[glutaryl-(S)-valine benzyl ester] was subjected to catalytic hydrogenolysis. Purification of the crude product obtained gave buprenorphine-[glutaryl-(S)-valine]ester as a white solid as shown below:

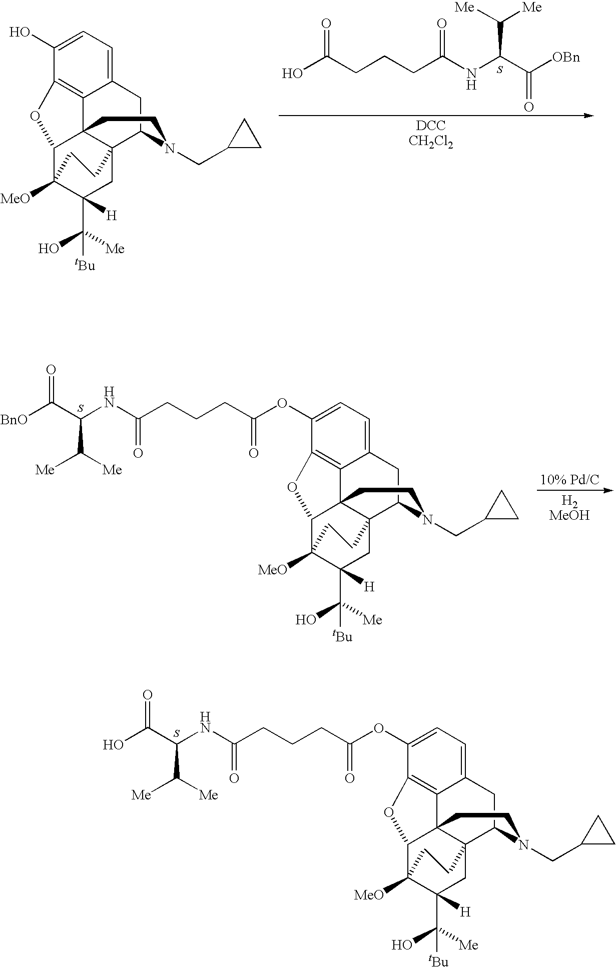

### Synthesis of Buprenorphine-[glutaryl-(S)-valine]ester

Details of Preparation glutaryl-(S)-valine ester

To a suspension of (S)-valine benzyl ester hydrochloride (1.01 g, 4.14 mmol), and glutaric anhydride (0.52 g, 4.56 mmol) in anhydrous dichloromethane (30 mL) was added triethylamine dropwise (1.32 mL, 9.53 mmol). The reaction mixture was stirred at room temperature for 3 h before diluting with dichloromethane (100 mL). This was washed with 5% aqueous citric acid solution (2×100 mL) and brine (100 mL). The organic layer was dried (MgSO₄) and concentrated to give glutaryl-(S)-valine benzyl ester (1.21 g, 91%), as an oil.

¹H-NMR (DMSO-d₆, 300 MHz): 12.01 (br s, 1H, CO₂H), 8.13 (d, J=8.0 Hz, 1H, NH), 7.41-7.30 (m, 5H, 5×ArH), 5.17-5.07 (m, 2H, benzylic CH₂), 4.22-4.17 (m, 1H, valine α-CH), 2.23-2.18 (m, 4H, 2×glutaryl CH₂), 2.09-1.98 (m, 1H, valine β-CH), 1.76-1.66 (m, 2H, glutaryl CH₂), 0.87-0.83 (m, 6H, 2×valine CH₃).

HPLC indicated the purity to be 99%.

LCMS showed 321.82 (consistent with protonated ion MH⁺).

Details of Preparation of buprenorphine-[glutaryl-(S)-valine] ester

To a stirred solution of glutaryl-(S)-valine benzyl ester (1.04 g, 3.24 mmol) and buprenorphine free base (1.16 g, 2.49 mmol) in anhydrous dichloromethane (30 mL) was added N,N′-dicyclohexylcarbodiimide (0.72 g, 3.49 mmol) and the resulting suspension was stirred at room temperature overnight. The reaction mixture was filtered through Celite and concentrated. Purification by medium pressure column chromatography (eluent 2% methanol in dichloromethane; product R_{f} 0.76 in 10% methanol in dichloromethane) gave buprenorphine-[glutaryl-(S)-valine benzyl ester] ester (1.19 g, 62%), as a clear oil.

10% Palladium on carbon (350 mg) was cautiously wetted with ethyl acetate (2 mL) under an atmosphere of nitrogen. A solution of buprenorphine-[glutaryl-(S)-valine benzyl ester] ester (595 mg, 0.77 mmol) in anhydrous methanol (30 mL) was added to the reaction flask, which was evacuated. An atmosphere of hydrogen was introduced via a balloon and the reaction was stirred overnight. The reaction mixture was filtered through Celite and concentrated to a white solid. Purification by medium pressure column chromatography (eluent 4% methanol in dichloromethane; product R_{f} 0.30 in 10% methanol in dichloromethane) gave buprenorphine-[glutaryl-(S)-valine]ester (296 mg, 56%), as an off-white solid.

¹H-NMR (DMSO-d₆, 300 MHz): 12.45 (br s, 1H, CO₂H), 7.88 (d, J=8.5 Hz, 1H, amide NH), 6.73 (d, J=8.1 Hz, 1H, ArH), 6.54 (d, J=8.1 Hz, 1H, ArH), 5.37 (s, 1H, CHO), 4.35 (s, 1H, CHN), 4.07-4.03 (m, 1H, valine α-CH), 3.27 (s, 3H, OCH₃), 2.91-2.85 (m, 2H, CH₂), 2.72-2.62 (m, 3H, CH₂+valine β-CH), 2.53-2.46 (m, 2H, CH₂), 2.35-2.04 (m, 4H, 2×CH₂), 1.98-1.77 (m, 4H, 2×CH₂), 1.69-1.49 (m, 5H, 2×CH₂+CH), 1.28-1.16 (m, 4H, CH+CH₃), 1.07-0.95 (m, 2H, CH₂), 0.86 (s, 9H, tert-butyl), 0.77 (d, J=6.8 Hz, 6H, 2×valine CH₃), 0.45-0.29 (m, 2H, 2×cyclopropyl CH), 0.06-0.05 (m, 2H, 2×cyclopropyl CH).

HPLC indicated the purity to be 99%.

LCMS showed 680.87 (consistent with protonated ion MH⁺).

### Example 16

### In vitro Stability of Dicarboxylate Amino Acid Ester Prodrugs Under Conditions Prevailing in the Gut

### Methodology

Inherent chemical and biological stability of the prodrugs of the present invention, in the conditions prevailing in the GI tract, is an important requirement. If a prodrug is prematurely hydrolyzed, the gut opioid receptors will be exposed to the parent active drug (e.g., oxycodone, codeine, dihydrocodeine) and consequently, a reduction in gut motility will occur. Additionally, premature hydrolysis of the prodrug would reduce the opportunity for improvement in bioavailability and continuous generation of the opioid from the prodrug. Therefore, systemic delivery of the active agent would be precluded.

To investigate if the prodrugs of the present invention are stable in conditions mimicking the gut, various oxycodone, codeine, and dihydrocodeine dicarboxylate amino acid enolesters, were incubated at 37° C. in simulated gastric and simulated intestinal juice (USP defined composition) for 2 hours. The remaining concentrations of the prodrug were then assayed by HPLC.

### Results

As can be seen in Table 9, all of these conjugates tended to be very stable under the stimulated conditions existing in the GI tract. Thus, these compounds would be expected to be absorbed intact and to have no direct effect on the opioid receptors in the gut.

TABLE 9
Dicarboxylate Linked Prodrug Stability in Various Media

| | Simulated | Simulated | | |
|---|---|---|---|---|
| | gastric fluid | intestinal fluid | Distilled water | |
| | (pH 1.1): % | (pH 6.8): % | (pH 5.9): % | pH 10.0 buffer: |
| | remaining | remaining after | remaining | % remaining |
| Compound | after 2 h/37° C. | 2 h/37° C. | after 2 h/20° C. | after 2 h/20° C. |
| Oxycodone-[succinyl-(S)-valine] | 98 | 85 | 100 | 64 |
| enol ester | | | | |
| Codeine-[succinyl-(S)-valine] | 99 | 98 | 100 | 92 |
| ester | | | | |
| Dihydrocodeine-[succinyl-(S)- | 100 | 100 | 100 | 98 |
| valine] ester | | | | |
| Oxycodone-Succinyl-Leu Enol | 98 | 78 | 100 | 68 |
| Ester | | | | |
| Oxycodone-Succinyl-Ile Enol | 98 | 91 | 100 | 66 |
| Ester | | | | |
| Oxycodone-Succinyl-Phe Enol | 99 | 3 | 100 | 65 |
| Ester | | | | |
| Oxycodone-Succinyl-Met Enol | 99 | 84 | 100 | 58 |
| Ester | | | | |
| Oxycodone-Succinyl-Pro Enol | 98 | 96 | 100 | 80 |
| Ester | | | | |
| Oxycodone-Glutaryl-Val Enol | 98 | 87 | 100 | 75 |
| Ester | | | | |
| Oxycodone-Glutaryl-Ile Enol | 98 | 80 | 100 | 80 |
| Ester | | | | |
| Oxycodone-Glutaryl-Leu Enol | 98 | 79 | — | — |
| Ester | | | | |

### Example 17

### Bioavailability of Oxycodone, Codeine and Dihydrocodeine from their Respective Succinyl Valine Ester Prodrugs in the Dog

Three sets of test substances (i.e., (1) codeine and codeine succinyl valine ester; (2) oxycodone and oxycodone succinyl valine ester and (3) dihydrocodeine and dihydrocodeine succinyl valine ester) were administered by oral gavage to three separate groups of five dogs in a two-way crossover design (i.e., each set of dogs were administered one opioid, and its respective succinyl ester linked prodrug). The characteristics of the test animals are set out in Table 10, below.

TABLE 10
Characteristics of Experimental Dogs Used in Study

| | |
|---|---|
| Species | Dog |
| Type | Beagle |
| Number and sex | 5 males |
| Approximate age | 3-4 months at the start of treatment |
| Approx. bodyweight | 7-9 kg at the start of treatment |
| Source | Huntingdon Life Sciences stock |

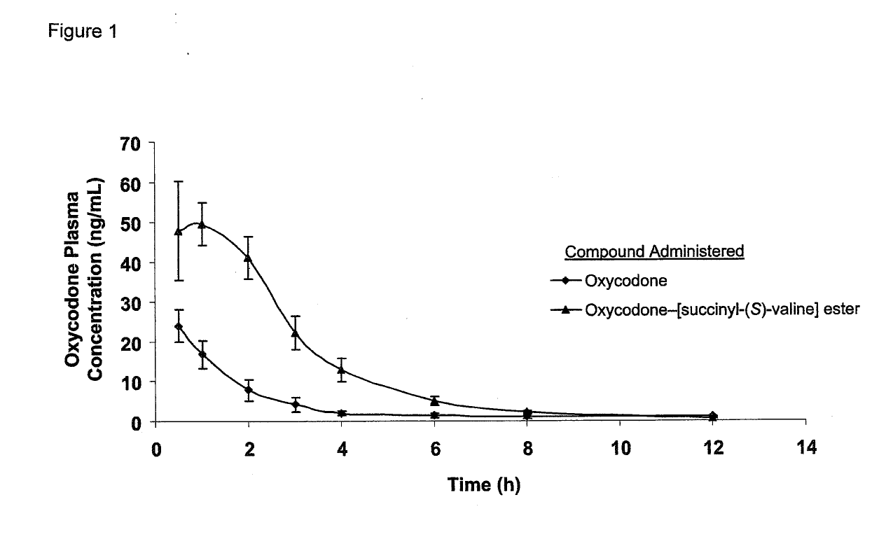

Blood samples were taken at various times after administration and submitted to analysis for the parent drug and pro-drug using a validated LC-MS-MS assay. Pharmacokinetic parameters derived from the plasma analytical data were determined using Win Nonlin. The results are given in Tables 11-13, and shown graphically in FIGS. 1-3.

### Oxycodone Results

TABLE 11
Pharmacokinetics of Oxycodone in Dogs After Oral Administration
of Oxycodone HCl (1 mg/kg) or Oxycodone-[Succinyl-(S)-Valine]
Enol Ester at 1 mg Free Base Equivalents Oxycodone/kg

| Oxycodone HCl | | | | | | | |
|---|---|---|---|---|---|---|---|
| Pharmacokinetic | Dog No. | | | | | | |
| parameter | 1 | 2 | 3 | 4 | 5 | Mean | sd |
| Cₘₐₓ (ng/mL) | 27.7 | 28.9 | 22.3 | 21.3 | 19.9 | 22.3 | 4.0 |
| Tₘₐₓ (h) | 0.5 | 0.5 | 0.5 | 0.5 | 0.5 | 0.5^{a} | — |
| AUCₜ (ng · h/mL) | 50.1 | 48.6 | 31.3 | 50.3 | 32.3 | 42.5 | 9.81 |
| AUC (ng · h/mL) | 61.6 | 52.8 | 32.6 | 51.8 | 34.2 | 46.6 | 12.7 |
| t½ (h) | 7.60 | 2.20 | 1.30 | 1.50 | 1.00 | 2.72 | 2.76 |
| T_{>50% Cmax} (h) | 0.5 | 0.5 | 0.5 | 0.5 | 0.5 | 0.5^{a} | |

| Oxycodone- | | | | | | | |
|---|---|---|---|---|---|---|---|
| [succinyl-(S)- | | | | | | | |
| valine] enol | | | | | | | |
| ester | | | | | | | |
| Pharmacokinetic | Dog No. | | | | | | |
| parameter | 1 | 2 | 3 | 4 | 5 | Mean | sd |
| Cₘₐₓ (ng/mL) | 52.0 | 55.5 | 43.8 | 49.6 | 66.5 | 52.0 | 8.4 |
| Tₘₐₓ (h) | 0.5 | 1 | 0.5 | 1 | 0.5 | 0.5^{a} | — |
| AUCₜ (ng · h/mL) | 158 | 182 | 160 | 149 | 150 | 160 | 13.3 |
| AUC (ng · h/mL) | 159 | 183 | 161 | 153 | 154 | 162 | 12.2 |
| t½ (h) | 1.70 | 1.80 | 1.80 | 1.70 | 1.60 | 1.72 | 0.08 |
| T_{>50% Cmax} (h) | 1.5 | 1.5 | 2.5 | 2.5 | 1.5 | 1.5^{a} | |
| F (%) | 258 | 347 | 494 | 295 | 450 | 369 | 101 |

^{a}Median value

The pharmacokinetic advantages of oxycodone succinyl valine ester can be seen in Table 11 and FIG. 1. These data show peak plasma levels of oxycodone approximately two fold higher after an equimolar dose of the succinyl valine prodrug (as compared to oxycodone HCl) while systemic exposure expressed as AUC, was 3.5-fold greater and associated with a much smaller variability (relative standard deviation just 8% vs. 27% for oxycodone HCl).

While peak oxycodone plasma levels after giving the succinyl valine prodrug were still reached quickly, within 0.5 hour, thereby ensuring a rapid onset of action, oxycodone peak levels persisted for somewhat longer when the prodrug was administered. This was reflected by the period for which plasma concentrations were maintained above 50% of the Cₘₐₓ values, which was three times longer following administration of the succinic acid linked valine prodrug than after giving the drug itself. This phenomenon was observed when the succinyl valine prodrug was administered in dogs (FIG. 1). These results could represent a potential advantage for pain management with the succinic acid linked prodrug. The prodrug better maintains plasma drug concentrations, enabling less frequent dosing while still sustaining analgesia, and may be the result of continuing generation of the drug from a plasma reservoir of prodrug.

### Codeine Results

TABLE 12
Pharmacokinetic Parameters of Codeine in Dogs After Oral
Administration of Either Codeine HCl (1 mg/kg) or Codeine-
[Succinyl-(S)-Valine] Ester at 1 mg Free Base Equivalents Codeine/kg

| Codeine HCl | | | | | | | |
|---|---|---|---|---|---|---|---|
| Pharmacokinetic | Dog No. | | | | | | |
| parameter | 1 | 2 | 3 | 4 | 5 | Mean | sd |
| Cₘₐₓ (ng/mL) | 1.59 | 1.30 | 3.86 | 1.29 | 1.72 | 1.95 | 1.08 |
| Tₘₐₓ (h) | 0.5 | 0.5 | 0.5 | 0.5 | 0.5 | 0.5^{a} | |
| AUCₜ (ng · h/mL) | 3.74 | 2.51 | 8.97 | 3.53 | 3.85 | 4.52 | 2.54 |
| AUC (ng · h/mL) | 3.82 | 2.55 | 8.99 | 3.59 | 4.04 | 4.60 | 2.52 |
| t½ (h) | 1.4 | 0.8 | 1.9 | 0.9 | 1.8 | 1.2^{b} | |
| T_{>50%Cmax} (h) | 1.5 | 0.5 | 1.5 | 1.5 | 0.5 | 1.5^{a} | |
| F^{c} (%) | 1.59 | 1.30 | 3.86 | 1.29 | 1.72 | 1.95 | 1.08 |
| Codeine-[succinyl- | | | | | | | |
| (S)-valine] ester | | | | | | | |
| Pharmacokinetic | Dog No. | | | | | | |
| parameter | 1 | 2 | 3 | 4 | 5 | Mean | sd |
| Cₘₐₓ (ng/mL) | 8.29 | 5.52 | 5.54 | 6.62 | 12.0 | 7.59 | 2.71 |
| Tₘₐₓ (h) | 2 | 2 | 3 | 2 | 3 | 2^{a} | |
| AUCₜ (ng · h/mL) | 27.0 | 18.3 | 15.5 | 19.9 | 41.4 | 24.4 | 10.4 |
| AUC (ng · h/mL) | 27.5 | 18.3 | 16.3 | 20.7 | 42.5 | 25.1 | 10.6 |
| t½ (h) | 1.2 | 0.4 | 1.0 | 2.8 | 1.2 | 0.9^{b} | |
| T_{>50%Cmax} (h) | 2 | 2 | 1 | 2 | 2 | 2^{a} | |
| F^{c} (%) | 720 | 718 | 181 | 577 | 1050 | 649 | 315 |

^{a}Median value
^{b}Calculated as ln2/mean k
^{c}Relative bioavailability

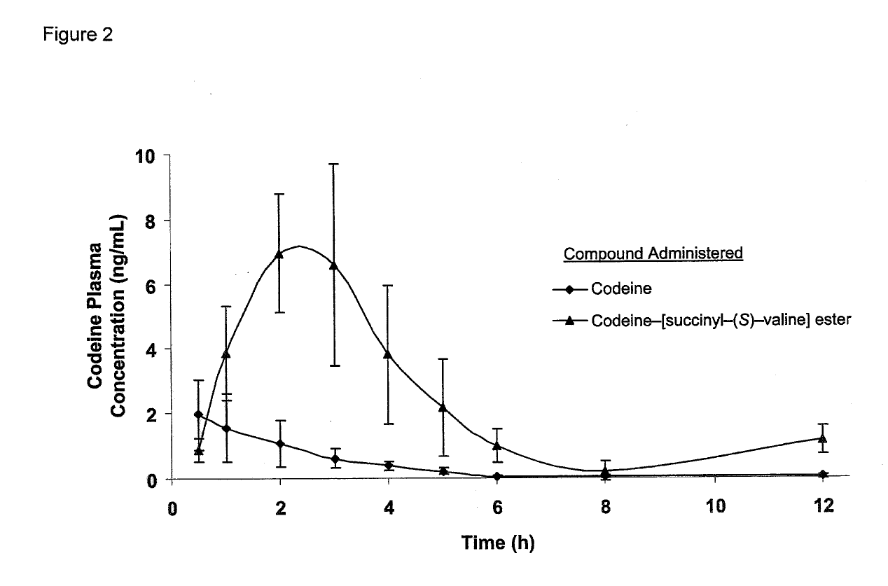

As seen in Table 12 and FIG. 2, administration of codeine succinyl valine ester resulted in Tₘₐₓ for the resultant codeine occurring later than after giving the parent drug (2 hour vs. 0.5 hour). Overall exposure to codeine after giving the prodrug was much greater, with a mean relative bioavailability (AUC) of 6.5 fold over that after giving codeine itself. There was some evidence for greater persistence of drug in plasma after giving the prodrug—a T₅₀% Cₘₐₓ value (the period for which plasma drug levels remained above 50% of the Cₘₐₓ) increased from 1.2 hour (codeine) to 2 hour (codeine succinyl valine ester).

### Dihydrocodeine Results

TABLE 13
Pharmacokinetic Parameters of Dihydrocodeine in Dogs After Oral
Administration of Either Dihydrocodeine or Dihydrocodeine-[Succinyl-
(S)-Valine] Ester at 1 mg Free Base Equivalents Dihydrocodeine/kg

| Dihydrocodeine | Dog No. | | | | | | |
|---|---|---|---|---|---|---|---|
| Pharmacokinetic parameter | 1 | 2 | 3 | 4 | 5 | Mean | sd |
| Cₘₐₓ (ng/mL) | 5.29 | 11.6 | 1.29 | 24.9 | 9.27 | 10.4 | 9.0 |
| Tₘₐₓ (h) | 0.5 | 0.5 | 0.5 | 0.5 | 0.5 | 0.5^{a} | |
| AUCₜ (ng · h/mL) | 9.95 | 30.3 | 3.27 | 49.4 | 14.7 | 21.4 | 18.6 |
| AUC (ng · h/mL) | 10.2 | 31.0 | c | 51.3 | c | 30.8 | 20.6 |
| t½ (h) | 2.3 | 4.9 | c | 7.4 | c | 3.9^{b} | |
| T_{>50%Cmax} (h) | 0.5 | 0.5 | 0.5 | 0.5 | 0.5 | 0.5^{a} | |
| Dihydrocodeine- | | | | | | | |
| [succinyl- | | | | | | | |
| (S)-valine] ester | Dog No. | | | | | | |
| Pharmacokinetic parameter | 1 | 2 | 3 | 4 | 5 | Mean | sd |
| Cₘₐₓ (ng/mL) | 1.88 | 6.53 | 9.38 | 6.56 | 6.60 | 6.19 | 2.70 |
| Tₘₐₓ (h) | 3 | 2 | 2 | 2 | 2 | 2^{a} | |
| AUCₜ (ng · h/mL) | 8.73 | 29.3 | 35.5 | 35.4 | 21.2 | 26.0 | 11.3 |
| AUC (ng · h/mL) | d | 30.1 | 36.4 | 35.6 | d | 27.0 | 11.0 |
| t½ (h) | d | 5.7 | 2.1 | 3.5 | d | 3.2^{b} | |
| T_{>50%Cmax} (h) | 3 | 2 | 3 | 1 | 1 | 2^{a} | |
| F^{c} (%) | 88 | 100 | 1319^{e} | 62 | 144 | 99 | 34 |

^{a}Median value
^{b}Calculated as ln2/mean k
^{c}The regression coefficient was ≦0.7
^{d}not calculable

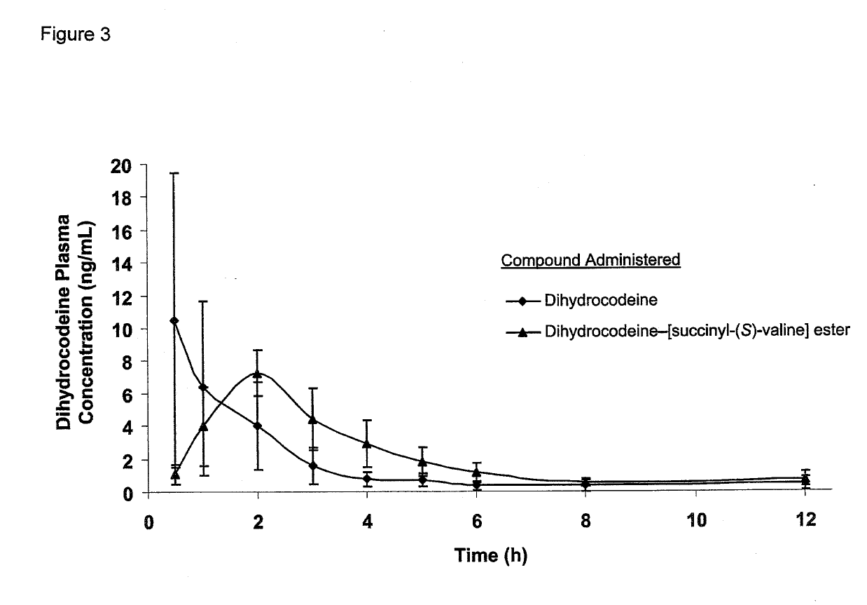

The results for dihydrocodeine are presented graphically in FIG. 3 as well as in Table 13. The data show comparable systemic availability of dihydrocodeine after giving either the drug itself or the prodrug, although there was less variability in dihydrocodeine plasma levels after administering the prodrug. For example the variability in exposure (expressed as relative standard deviation around the AUC) after administering the parent drug molecule was 67%, compared to 30% after giving the prodrug. Furthermore, dihydrocodeine persisted for longer in plasma after giving the prodrug, with T₅₀% Cₘₐₓ values of 2 h for the prodrug, and 0.5 h for the parent compound. This greater persistence may result in less frequent clinical administration and consequently improved patient compliance.

### Example 18

### Ex vivo Assessment of the Effects of Oxycodone, Codeine, Dihydrocodeine and their Respective Succinyl Valine Ester Prodrugs on Smooth Muscle Contractility in Isolated Guinea Pit Small Intestine

### Methodology

Strips of guinea pig small intestine myenteric plexus longitudinal muscle were mounted between platinum ring electrodes. The tissue was stretched to a steady tension of about 1 g and changes in force production were recorded using sensitive transducers.

Optimal voltage for stimulation was determined while the tissue was paced with an electrical field stimulation (EFS) at 14 Hz, with a pulse width of 0.5 msec. (Trains of pulses then continued for 20 seconds, every 50 seconds).

EFS at optimal voltage continued throughout the protocol (stable responses=“baseline measurement of EFS”).

The test conditions employed were as follows:

For the opioid comparison:

(1) Vehicle (deionized water, added at equivalent volume additions to test articles);
(2) Oxycodone at 6 concentrations (10 nM, 100 μM, 1 μM, 3 μM, 10 μM, 30 μM) and
(3) Oxycodone succinyl valine ester at 6 concentrations (10 nM, 100 nM, 1 μM, 3 μM, 10 μM, 30 μM)

For the codeine comparison:

(1) Vehicle (deionized water, added at equivalent volume additions to test articles);
(2) Codeine at 6 concentrations (100 nM, 1 μM, 3 μM, 10 μM, 30 μM, 100 μM) and
(3) Codeine succinyl valine ester at 6 concentrations (100 nM, 1 μM, 3 μM, 10 μM, 30 μM, 100 μM)

For the dihydrocodeine comparison:

(1) Vehicle (deionized water, added at equivalent volume additions to test articles);
(2) Dihydrocodeine at 6 concentrations (100 nM, 1 μM, 3 μM, 10 μM, 30 μM, 100 μM) and
(3) Dihydrocodeine succinyl valine ester at 6 concentrations (100 nM, 1 μM, 3 μM, 10 μM, 30 μM, 100 μM)

Following 10 minutes of baseline EFS, the first addition of test article or vehicle (deionized water) was performed.

Test concentrations were added in a non-cumulative manner with PSS washes between each addition. Next, TTX (Na+ channel blocker) was added to confirm EFS responses were elicited via nerve stimulation. EFS was then stopped.

### Oxycodone Results

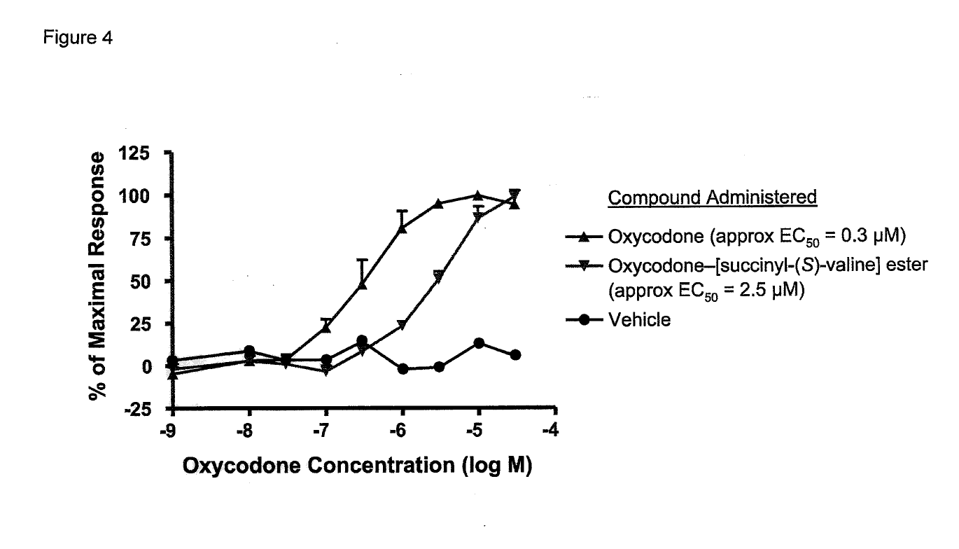

These results shown in FIG. 4 reveal a dramatic 10-fold reduction in the opioid effects of the oxycodone succinyl valine ester on guinea pig ileal smooth muscle compared to oxycodone itself. The respective EC₅₀ values were 2 μM and 0.2 μM suggesting a potential for much less opioid mediated inhibitory effects of the oxycodone prodrug on gut motility. On this basis, it would be expected that oxycodone succinyl valine ester would have a much lower potential to cause constipation than the drug itself.

### Codeine Results

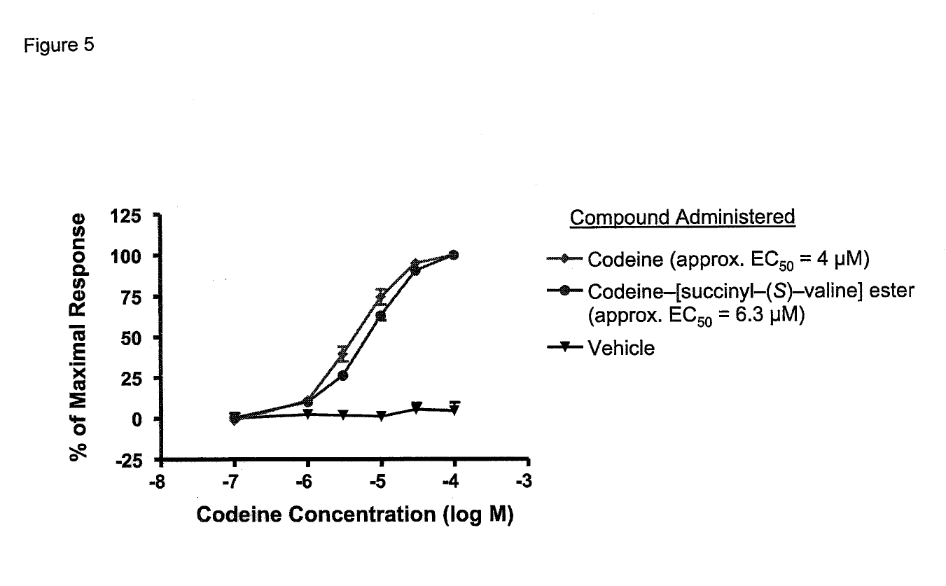

The results shown in FIG. 5 showed an increase in EC₅₀ for the opioid effects of codeine succinyl valine ester on ileal smooth muscle, compared to codeine itself (6.3 μM for the prodrug, compared to 4.0 μM oxycodone itself, a 50% reduction in potency). This result suggests a reduced potential for opioid mediated inhibitory effects on gut motility and therefore, a lower potential for the codeine prodrug to cause constipation, as compared to codeine itself.

### Dihydrocodeine Results

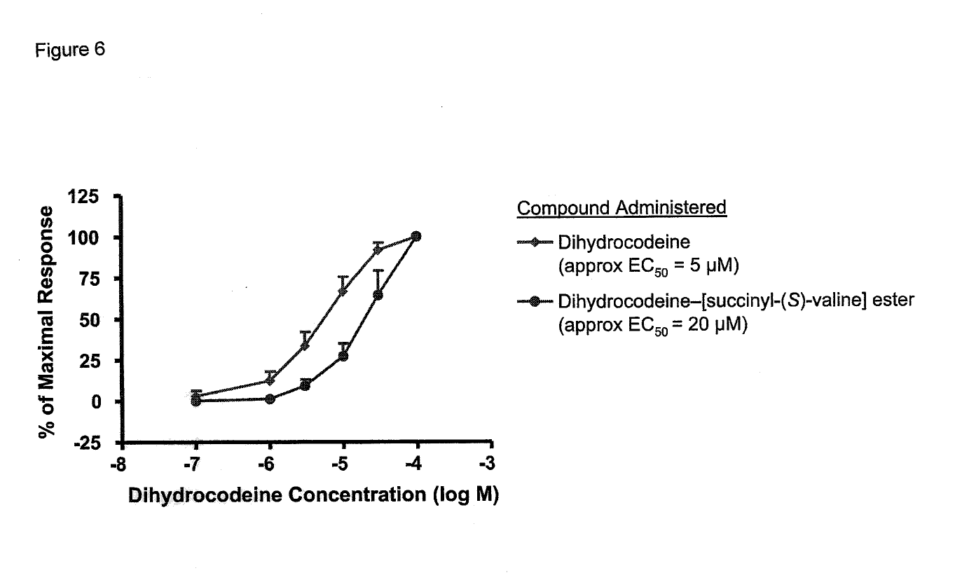

The results presented in FIG. 6 show a significant reduction in the opioid effects of the dihydrocodeine succinyl valine ester compared to the parent drug on ileal smooth muscle compared. The EC₅₀ values for the prodrug and drug itself were 20 μM and 5.0 μM, respectively. Again, this suggests a potential for much less opioid mediated inhibitory effects of the dihydrocodeine prodrug on gut motility. On this basis it would be expected that dihydrocodeine succinyl valine ester would have a lower potential to cause constipation than dihydrocodeine itself.

### Example 19

### In vivo Effects of Oxycodone, Codeine, Dihydrocodeine and Their Respective Succinyl Valine Ester Pro-Drugs on Gut Motility in the Rat

### Methodology

The effects of oxycodone, codeine, dihydrocodeine and their respective succinyl valine ester pro-drugs on GI motility were assessed by means of the charcoal propulsion test. Test treatments were administered to groups of up to 10 rats fasted overnight prior to the test.

The method used was based on that described by Takemori et al. (Takemori et al. (1969). J. Pharmacol. Exp. Ther. 169, 39). Test treatments were administered orally 60 minutes prior to a 2.0 mL oral dose of a 10% suspension of charcoal in 5% gum arabic. Thirty minutes after dosing with charcoal, the rats were sacrificed and the entire gastro-intestinal tract quickly and carefully removed. The distance the charcoal meal had traveled from the pyloric sphincter toward the caecum was measured and expressed as a percentage of both the total gut length and the length of the small intestine.

### Oxycodone Results

The results presented in Table 14 show that oxycodone itself elicited a profound effect on gut motility, delaying the passage of the charcoal meal after a 30 mg/kg dose by 52%. In contrast, oxycodone succinyl valine ester, after the same equimiolar dose, delayed gut motility by only ˜16%. These data suggest that the succinyl valine ester of oxycodone is considerably less constipating in man than is the parent drug molecule.

TABLE 14
Effects of Oral Administration of Oxycodone and Oxycodone-[Succinyl-(S)-Valine]
Ester on Gastrointestinal Motility in the Female Rat

| | | Group mean distance | | | |
|---|---|---|---|---|---|
| | | travelled by charcoal as % | | % change from | |
| | | of (±sd) | | vehicle-treated animals | |
| | Dose | Small | | Small | |
| Oral treatment | (mg/kg) | intestine | Total gut length | intestine | Total gut length |
| Vehicle | — | 79.9 ± 5.9 | 68.0 ± 4.9 | — | — |
| (sterile water) | | | | | |
| Oxycodone | 10 | 67.4** ± 9.7 | 57.1** ± 8.4 | −15.6 | −16.0 |
| Oxycodone | 30 | 38.2** ± 13.2 | 32.5** ± 11.3 | −52.2 | −52.2 |
| Oxycodone | 100 | 18.76** ± 5.2 | 16.0** ± 4.4 | −75.5 | −76.5 |
| Oxycodone-[succinyl-(S)- | 10 | 70.2* ± 9.0 | 60.4* ± 8.1 | −12.1 | −11.2 |
| valine] ester | | | | | |
| Oxycodone-[succinyl-(S)- | 30 | 67.3**⁺⁺ ± 8.6 | 57.4**⁺⁺ ± 7.2 | −15.8 | −15.6 |
| valine] ester | | | | | |
| Oxycodone-[succinyl-(S)- | 100 | 51.6**⁺⁺ ± 12.3 | 44.2**⁺⁺ ± 10.8 | −35.4 | −35.0 |
| valine] ester | | | | | |

Statistical significance of difference from vehicle-treated group: *p < 0.05 **p < 0.01
Statistical significance of difference from parent drug treated group: ⁺⁺p < 0.01

### Codeine Results

The results presented in Table 15 show that codeine itself elicited a marked effect on gut motility, delaying the passage of the charcoal meal after a 30 mg/kg dose by greater than 30%. In contrast, codeine succinyl valine ester, after an equimolar dose, delayed gut motility by less than half the amount of codeine. These data suggest that the succinyl valine ester of codeine is significantly less constipating in man than is the parent drug molecule.

TABLE 15
Effects of Oral Administration of Codeine and Codeine Succinyl Valine Ester on
GastroIntestinal Motility in the Rat

| | | Group mean distance | | | |
|---|---|---|---|---|---|
| | | travelled by charcoal as | | % change from | |
| | | % of (±sd) | | vehicle-treated animals | |
| | Dose | Small | | Small | |
| Oral treatment | (mg/kg) | intestine | Total gut length | intestine | Total gut length |
| Vehicle | — | 76.4 ± 5.51 | 65.4 ± 4.55 | — | — |
| (sterile water) | | | | | |
| Codeine phosphate | 10 | 59.6** ± 8.86 | 51.2** ± 7.54 | −22.0 | −21.7 |
| hemihydrate | | | | | |
| Codeine phosphate | 30 | 51.9** ± 19.44 | 44.5** ± 16.66 | −32.1 | −32.0 |
| hemihydrate | | | | | |
| Codeine phosphate | 100 | 49.6** ± 17.76 | 42.6** ± 15.36 | −35.1 | −34.9 |
| hemihydrate | | | | | |
| Codeine-[succinyl-(S)-valine] | 10 | 69.0* ± 4.98 | 59.3* ± 4.32 | −9.7 | −9.3 |
| ester | | | | | |
| Codeine-[succinyl-(S)-valine] | 30 | 64.4** ± 6.36 | 55.5** ± 5.14 | −15.7 | −15.1 |
| ester | | | | | |
| Codeine-[succinyl-(S)-valine] | 100 | 63.9** ± 9.14 | 55.2** ± 8.08 | −16.4 | −15.6 |
| ester | | | | | |

Statistical significance of difference from vehicle-treated group: *p < 0.05 **p < 0.01

### Dihydrocodeine Results

The results presented in Table 16 show that dihydrocodeine itself elicited a significant effect on gut motility delaying the passage of the charcoal meal after a 30 mg/kg dose by ˜30%. By contrast dihydrocodeine succinyl valine ester after an equimolar dose delayed gut motility by only 8.5%. These data suggest that the succinyl valine ester of dihydrocodeine is considerably less constipating in man than is the parent drug molecule.

TABLE 16
Effects of Oral Administration of Dihydrocodeine Hydrogen Tartrate and
Dihydrocodeine-[Succinyl-(S)-Valine] Ester on Gastrointestinal Motility in the Rat

| | | Group mean distance | | | |
|---|---|---|---|---|---|
| | | travelled by charcoal as | | % change from | |
| | | % of (± sd) | | vehicle-treated animals | |
| | Dose | Small | Total gut | Small | |
| Oral treatment | (mg/kg) | intestine | length | intestine | Total gut length |
| Vehicle (Sterile water) | — | 73.4 ± 7.32 | 61.9 ± 5.98 | — | — |
| Dihydrocodeine Hydrogen | 10 | 60.5** ± 10.75 | 51.8** ± 9.45 | −17.6 | −16.3 |
| Tartrate | | | | | |
| Dihydrocodeine Hydrogen | 30 | 51.5** ± 10.89 | 43.0** ± 9.11 | −29.8 | −30.5 |
| Tartrate | | | | | |
| Dihydrocodeine Hydrogen | 100 | 32.0** ± 15.57 | 26.6** ± 12.89 | −56.4 | −57.0 |
| Tartrate | | | | | |
| Dihydrocodeine Hydrogen | 300 | 23.7** ± 11.53 | 19.8** ± 9.71 | −67.7 | −68.0 |
| Tartrate | | | | | |
| Dihydrocodeine-[succinyl-(S)-valine] | 10 | 61.3* ± 6.41 | 52.9* ± 5.60 | −17.4 | −16.6 |
| ester | | | | | |
| Dihydrocodeine-[succinyl-(S)-valine] | 30 | 67.9*⁺⁺ ± 11.01 | 58.0*⁺⁺ ± 8.88 | −8.5 | −8.5 |
| ester | | | | | |
| Dihydrocodeine-[succinyl-(S)-valine] | 100 | 64.3*⁺⁺ ± 7.36 | 55.2*⁺⁺ ± 6.57 | −13.3 | −12.9 |
| ester | | | | | |
| Dihydrocodeine-[succinyl-(S)-valine] | 300 | 47.7** ± 7.11 | 41.0** ± 5.99 | −35.7 | −35.3 |
| ester | | | | | |

Statistical significance of difference from vehicle-treated group: *p < 0.05 **p < 0.01
Statistical significance of difference from equi-dose of dihydrocodeine hydrogen tartrate: ⁺⁺p < 0.
sd Standard deviation

### Example 20

### Comparative Oral Bioavailability of Oxycodone from Various Dicarboxylate Bridged Amino Acids Prodrugs in Dogs

### Methodology

Test substances i.e oxycodone or one of eight amino acid prodrugs were administered by oral gavage to a group of five dogs in a nine-way crossover design The characteristics of the test animals are set out in Table 17, below.

TABLE 17
Characteristics of Experimental Dogs Used in Study

| | |
|---|---|
| Species | Dog |
| Type | Beagle |
| Number and sex | 5 males |
| Approximate age | 3-4 months at the start of treatment |
| Approx. bodyweight | 7-9 kg at the start of treatment |
| Source | Huntingdon Life Sciences stock |

Blood samples were taken at various times after administration and submitted to analysis for the parent drug and pro-drug using a validated LC-MS-MS assay. Pharmacokinetic parameters derived from the plasma analytical data were determined using Win Nonlin.

### Results

These are shown in Table 18

TABLE 18
Comparison of PK Parameters for Oxycodone after Oral
Administration of Various Prodrugs to Dogs at 1 mg/kg
Oxycodone Free Base

| | Cₘₐₓ | T50% | AUC₀₋ₜ | |
|---|---|---|---|---|
| Test Compound | (ng/mL) | Cₘₐₓ (h.) | (h. * ng/mL) | F % |
| Oxycodone | 49.7 (±15.70) | 2.0 (±0.6) | 106.7 (±23.0) | 29.4 |
| hydrochloride | | | | |
| Oxycodone succinyl | 49.3 (±14.1) | 1.6 (±0.3) | 114.4 (±32.9) | 31.5 |
| valine ester | | | | |
| Oxycodone glutaryl | 38.7 (±10.6) | 2.9 (±0.3) | 123.2 (±27.6) | 33.9 |
| valine ester | | | | |
| Oxycodone succinyl | 60.9 (±15.0) | 1.9 (±0.2) | 142.5 (±28.4) | 39.3 |
| leucine ester | | | | |
| Oxycodone succinyl | 57.9 (±8.7) | 1.6 (±0.4) | 130.6 (±25.6) | 36.0 |
| isoleucine ester | | | | |
| Oxycodone succinyl | 68.3 (±21.1) | 2.3 (±0.8) | 180.6 (±37.7) | 49.8 |
| methionine ester | | | | |
| Oxycodone glutaryl | 56.4 (±8.5) | 3.4 (±0.7) | 188.7 (±36.0) | 52.0 |
| leucine | | | | |
| Oxycodone glutaryl | 46.8 (±15.0) | 2.8 (±0.2) | 135.7 (±27.9) | 37.4 |
| isoleucine | | | | |
| Oxycodone succinyl | 20.1 (±4.60) | 3.4 (±0.9) | 75.2 (±13.7) | 20.8 |
| proline ester | | | | |

Standard deviations results are shown in brackets
Exposure according to AUC₀₋ₜ has been calculated using T as the last quantifiable time point
Tₘₐₓ values are expressed as median results

These results reveal a wide range of absolute oral bioavailabilities of oxycodone from these various prodrugs ranging from 20.8% from the succinyl proline ester to 52% from the glutaryl leucine conjugate. This latter conjugate not only gave the best oral bioavailability but also gave the longest period of sustainment of plasma drug concentrations which, if mirrored in man, would lead to less frequent drug dosage and improved patient compliance.

### Example 21

### Comparative Pharmacokinetics of Oxycodone in the Cynomolgus Monkey after Oral Administration of Either Oxycodone, Oxycodone Succinyl Valine Ester or Oxycodone Glutaryl Leucine Ester

### Methodology

Test substances i.e., oxycodone, oxycodone succinyl valine ester or oxycodone glutaryl leucine ester were administered to a group of five male cynomolgus monkeys in a three way crossover design. The compounds were all given at 1 mg oxycodone base equivalents/kg.

Blood samples were taken at various times after administration and submitted to analysis for the parent drug and prodrug using a validated LC-MS/MS assay. Pharmacokinetic parameters derived from the plasma analytical data were determined using Win Nonlin.

### Results

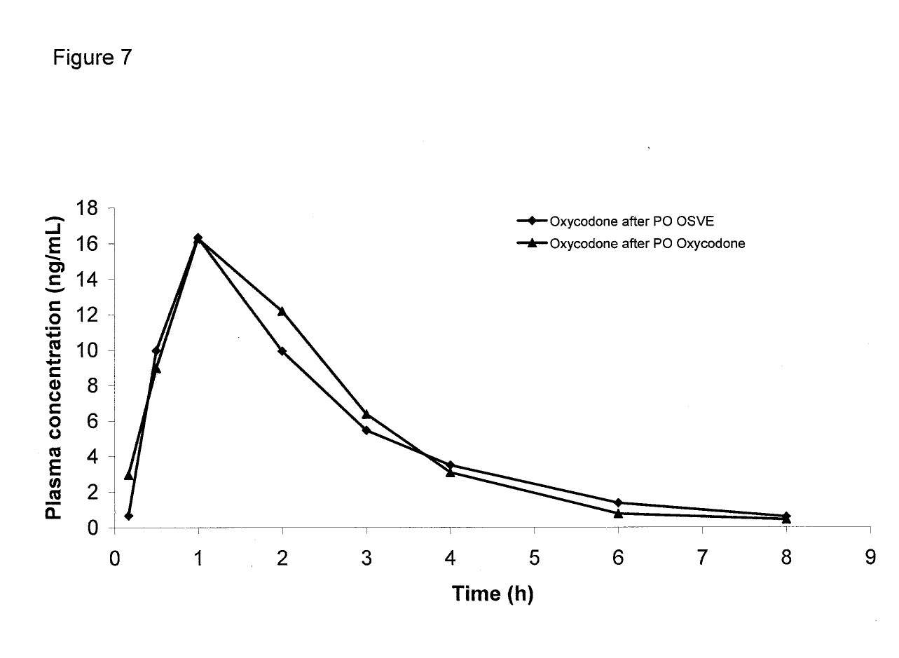

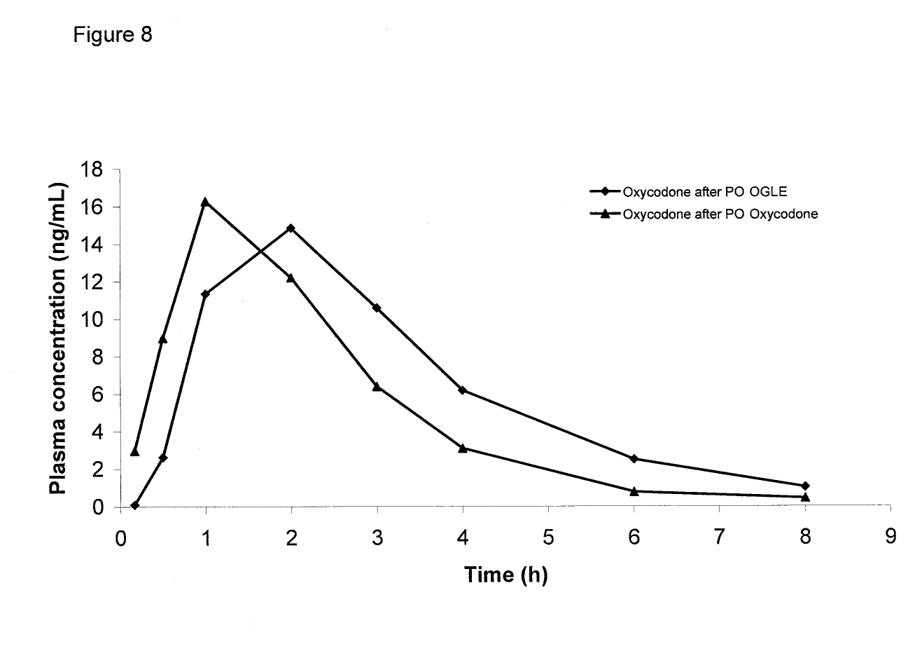

These are shown in Tables 19-21 and FIGS. 7-8

TABLE 19
Pharmacokinetics of Oxycodone in Male Cynomolgus Monkeys
Administered Oxycodone HCl by Oral Gavage at 1 mg Oxycodone
Free Base Equivalents/kg

| Oxycodone | | | | | | | |
|---|---|---|---|---|---|---|---|
| Pharmacokinetic | Monkey No. | | | | | | |
| parameter | 1 | 2 | 3 | 4 | 5 | Mean | sd |
| Cₘₐₓ (ng/mL) | 36.60 | 16.20 | 16.80 | 10.60 | 14.10 | 18.86 | 10.21 |
| Tₘₐₓ (h) | 1.00 | 0.50 | 1.00 | 2.00 | 1.00 | 1.10 | 0.55 |
| AUCₜ (ng · h/mL) | 83.96 | 25.37 | 39.00 | 30.23 | 30.05 | 41.72 | 24.12 |
| AUC (ng · h/mL) | 85.38 | 25.75 | 41.20 | 30.80 | 30.52 | 42.73 | 24.50 |
| t½ (h) | 1.23 | 0.99 | 1.50 | 1.09 | 0.91 | 1.14 | 0.23 |
| T_{>50%Cmax} (h) | 1.84 | 1.18 | 1.89 | 2.37 | 1.79 | 1.81 | 0.42 |

TABLE 20
Pharmacokinetics of Oxycodone and Ooxycodone Succinyl-(S)-
Valine Enol Ester in Male Cynomolgus Monkeys Orally Dosed with
Oxycodone Succinyl-(S)-Valine] Enol Ester at 1 mg Oxycodone
Free Base Equivalents/kg

| Oxycodone | | | | | | | |
|---|---|---|---|---|---|---|---|
| Pharmacokinetic | Monkey No. | | | | | | |
| parameter | 1 | 2 | 3 | 4 | 5 | Mean | sd |
| Cₘₐₓ (ng/mL) | 44.20 | 12.10 | 8.37 | 5.34 | 18.10 | 17.62 | 15.60 |
| Tₘₐₓ (h) | 1.00 | 0.50 | 2.00 | 2.00 | 1.00 | 1.30 | 0.67 |
| AUCₜ (ng · h/mL) | 88.46 | 24.93 | 31.30 | 25.04 | 32.13 | 40.37 | 27.09 |
| AUC (ng · h/mL) | 89.96 | 25.72 | 33.67 | 29.56 | 32.69 | 42.32 | 26.81 |
| t½ (h) | 1.33 | 1.13 | 1.82 | 2.47 | 0.96 | 1.54 | 0.61 |
| T_{>50%Cmax} (h) | 1.54 | 1.63 | 3.45 | 4.43 | 1.38 | 2.49 | 1.37 |
| Relative F (%) | 105% | 100% | 82% | 96% | 107% | 98% | 10% |
| Oxycodone [succinyl- | | | | | | | |
| (S)-valine] enol ester | | | | | | | |
| Pharmacokinetic | | | | | | | |
| parameter | 1 | 2 | 3 | 4 | 5 | Mean | sd |
| Cₘₐₓ (ng/mL) | 1.29 | 2.92 | 0.57 | 0.42 | 1.02 | 1.24 | 1.00 |
| Tₘₐₓ (h) | 0.5 | 0.5 | 0.5 | 0.5 | 0.5 | 0.5 | 0 |
| AUCₜ (ng · h/mL) | 0.76 | 1.70 | 0.39 | 0.32 | 0.71 | 0.78 | 0.55 |

TABLE 21
Pharmacokinetics of Oxycodone and Oxycodone Glutaryl-(S)-
Leucine Enol Ester in Male Cynomolgus Monkeys Orally Administered
Oxycodone Glutaryl-(S)-Leucine Enol Ester TFA at 1 mg
Oxycodone Free Base Equivalents/kg

| Oxycodone | | | | | | | |
|---|---|---|---|---|---|---|---|
| Pharmacokinetic | Monkey No. | | | | | | |
| parameter | 1 | 2 | 3 | 4 | 5 | Mean | sd |
| Cₘₐₓ (ng/mL) | 29.10 | 10.30 | 13.10 | 6.71 | 26.30 | 17.10 | 9.99 |
| Tₘₐₓ (h) | 2.00 | 1.00 | 3.00 | 2.00 | 1.00 | 1.80 | 0.84 |
| AUCₜ (ng · h/mL) | 84.60 | 30.76 | 47.38 | 31.51 | 57.87 | 50.42 | 22.23 |
| AUC (ng · h/mL) | 88.33 | 32.79 | 48.48 | 37.27 | 58.30 | 53.03 | 22.10 |
| t½ (h) | 1.48 | 1.87 | 1.15 | 2.48 | 1.01 | 1.60 | 0.60 |
| T_{>50%Cmax} (h) | 2.33 | 2.40 | 3.13 | 4.66 | 1.80 | 2.86 | 1.11 |
| Relative F (%) | 103% | 127% | 118% | 121% | 191% | 132% | 34% |
| Oxycodone glutaryl- | | | | | | | |
| (S)-leucine enol ester | | | | | | | |
| Pharmacokinetic | | | | | | | |
| parameter | 1 | 2 | 3 | 4 | 5 | Mean | sd |
| Cₘₐₓ (ng/mL) | BLQ | BLQ | BLQ | BLQ | BLQ | — | — |
| Tₘₐₓ (h) | — | — | — | — | — | — | — |
| AUCₜ (ng · h/mL) | — | — | — | — | — | — | — |

BLQ = below limitation of quantification

The pharmacokinetic profile of oxycodone after oral administration of the succinyl valine ester prodrug to monkeys (See FIG. 7) revealed comparable systemic availability to that seen after giving the parent drug with the AUC values being 42.3 and 42.7 ng/h/mL respectively. Cmax values were also very comparable being 18.9 and 17.1 ng/mL. The slightly lower Cmax after giving the prodrug may be a reflection of the altered concentrations time profile with greater plasma persistence seen after the prodrug T_{>50 % Cmax} 2.5 h vs 1.8 h.

Exposure to the prodrug (OSVE) was very low with Cmax values being only ˜7% of those of the active drug.

The pharmacokinetic profile of oxycodone after oral administration of the glutaryl leucine ester prodrug (OGLE) to monkeys (See FIG. 8) showed an even more encouraging profile. Total systemic exposure to oxycodone after the prodrug was 53 ng·h/mL compared to 42.7 ng·h/mL after giving the drug itself showing a modest 24% increase in bioavailability. The respective Cmaxvalues were comparable. However after the prodrug oxycodone persisted somewhat longer in plasma reflected by a T_{>50 % Cmax} 2.9 h cf 1.8 h after giving the drug itself. If this profile is maintained in man this should lead to less frequent dosing and greater compliance and patient convenience.

Prodrug levels after giving the glutaryl leucine ester prodrug were below the limit of quantification.

### Example 22

### Comparative Pharmacokinetics of Oxycodone after Oral Administration of Either Oxycodone Itself or Oxycodone Succinyl Valine Ester to Rats

### Methodology

Test substances i.e., oxycodone or oxycodone succinyl valine ester were administered by oral gavage to groups of female Sprague Dawley rats. The dose in both cases was 10 mg oxycodone free base equivalents/kg.

Blood samples were taken at various times after administration and submitted to analysis for the parent drug and prodrug using a validated LC-MS-MS assay. Pharmacokinetic parameters derived from the plasma analytical data were determined using Win Nonlin.

### Results

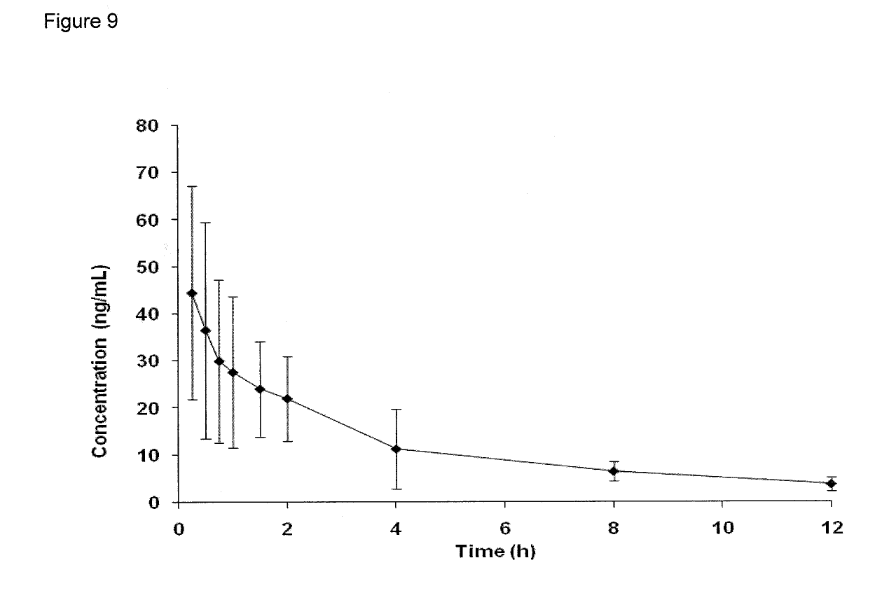

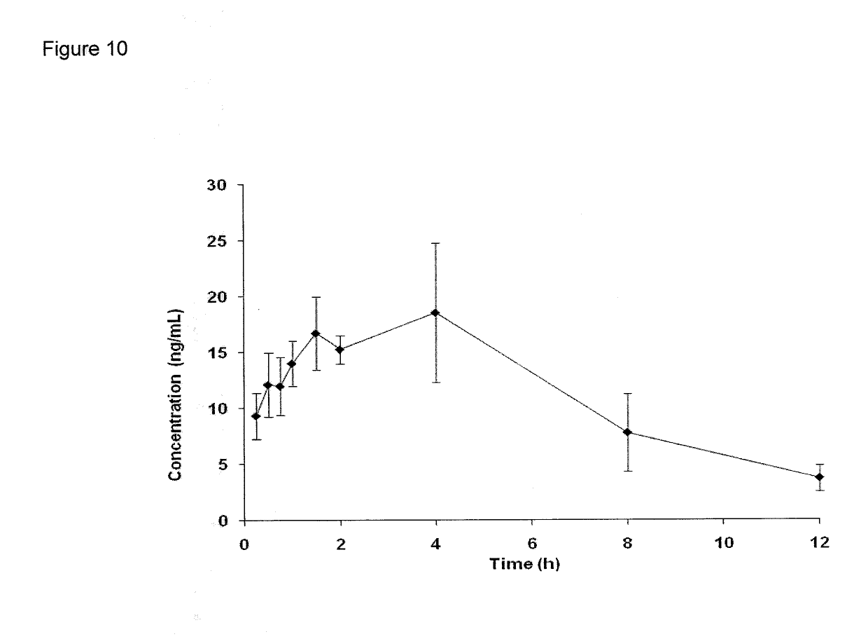

The results are given in Table 22-23 and FIGS. 9-10

TABLE 22
Pharmacokinetics of Oxycodone in Female Rats Orally Administered
Oxycodone Hydrochloride at 10 mg Oxycodone Free Base Equivalents/kg

| Pharmacokinetic | Rat No. | | | | | | |
|---|---|---|---|---|---|---|---|
| parameter | 6 | 7 | 8 | 9 | 10 | Mean | sd |
| Cₘₐₓ (ng/mL) | 36.8 | 73.2 | 33.7 | 26.0 | 64.3 | 46.8 | 20.7 |
| Tₘₐₓ (h) | 1.5 | 0.25 | 0.25 | 4 | 0.25 | 0.25^{a} | |
| AUCₜ (ng · h/mL) | 138 | 198 | 109 | 158 | 121 | 145 | 35 |
| AUC (ng · h/mL) | 148 | 230 | 135* | 175 | 129 | 171 | 44 |
| t½^{b} (h) | 2.96 | 4.49 | 5.12* | 3.12 | 3.15 | 3.43 | |
| T_{>50%Cmax} (h) | 1.75 | 0.75 | 0.5 | 1.75 | 0.75 | 0.75^{a} | |

^{a}Median value
^{b}Calculated as ln2/mean k
*Values excluded from mean calculations

TABLE 23
Pharmacokinetics of Oxycodone and Oxycodone Succinyl
Valine Enol Ester in Female Rats Orally Administered Oxycodone
[Succinyl-(S)-Valine] Enol Ester at 10 mg Oxycodone Free
Base Equivalents/kg

| Oxycodone | | | | | | | |
|---|---|---|---|---|---|---|---|
| Pharmacokinetic | Rat No. | | | | | | |
| parameter | 1 | 2 | 3 | 4 | 5 | Mean | sd |
| Cₘₐₓ (ng/mL) | 16.8 | 21.9 | 16.5 | 17.0 | 29.3 | 20.3 | 5.5 |
| Tₘₐₓ (h) | 1 | 1.5 | 1.5 | 1.5 | 4 | 1.5^{a} | |
| AUCₜ (ng · h/mL) | 148 | 128 | 130 | 118 | 151 | 135 | 14 |
| AUC (ng · h/mL) | 215* | 143 | 157 | 158 | 158 | 154 | 7 |
| t½ (h) | 7.56* | 3.55 | 4.66 | 5.78 | 2.26 | 3.59^{b} | |
| T_{>50%Cmax} (h) | 11.8 | 3.5 | 11.8 | 3.75 | 2 | 3.75^{a} | |
| Relative F (%) | 126 | 84 | 92 | 92 | 92 | 97 | 16 |
| Oxycodone succinyl | | | | | | | |
| valine enol ester | | | | | | | |
| Pharmacokinetic | | | | | | | |
| parameter | 1 | 2 | 3 | 4 | 5 | Mean | sd |
| Cₘₐₓ (ng/mL) | 6.77 | 2.00 | BLQ | 1.93 | 1.05 | 2.35 | 2.60 |
| Tₘₐₓ (h) | 0.25 | 1.5 | — | 0.25 | 0.50 | 0.375^{a} | |
| AUCₜ (ng · h/mL) | 3.53 | 2.55 | BLQ | BLQ | 0.131 | 1.24 | 1.68 |

^{a}Median value
^{b}Calculated as ln2/mean k
*Values excluded from mean calculations
BLQ Values taken as zero for calculation of the mean

These results show the prodrug resulted in attainment of a Cmax of ˜43% and an AUC of 90% of that seen after administration of the parent drug. The difference in Cmax but comparability in AUC may be largely explicable in terms of a slower attainment maximum plasma concentrations (Tmax 1.5 h vs 0.25 h) and a subsequent greater persistence in plasma (T_{>50 % Cmax} 3.75 h vs 0.75 h).

If this profile is maintained in man this should lead to less frequent dosing and greater compliance and patient convenience.

### Example 23

### Comparative Assessment of the Systemic Availability of Oxycodone after Intranasal Instillation of Either Oxycodone Itself or Oxycodone Succinyl Valine Esters to Dogs

### Methodology

Test substances i.e., oxycodone or oxycodone succinyl valine ester were administered by intranasal insufflation (using a Penn Century® DP-4 insufflator) to a group of five male beagle dogs in a crossover study design. The dose in both cases was approx 0.25 mg oxycodone free base equivalents/kg and particle size of the material was determined by light microscopy to be very comparable for both compounds.

Blood samples were taken at various times after administration and submitted to analysis for the parent drug and prodrug using a validated LC-MS-MS assay. Pharmacokinetic parameters derived from the plasma analytical data were determined using Win Nonlin.

### Results

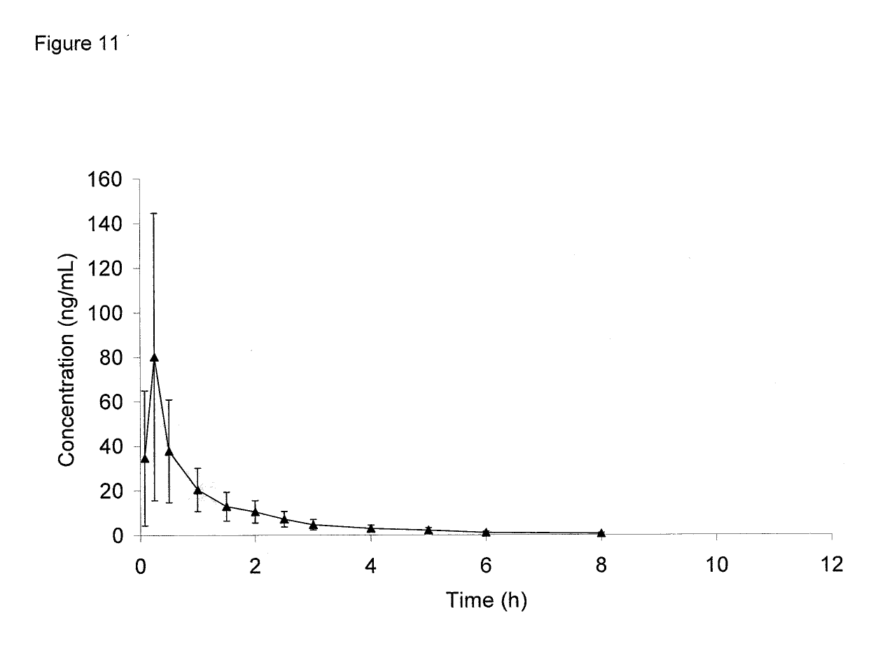

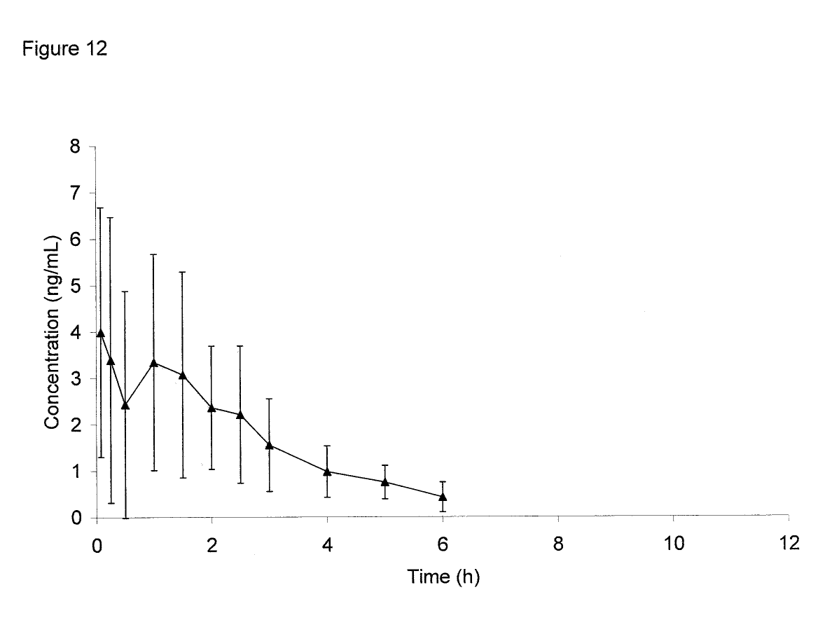

The results are given in Tables 24-25 and FIGS. 11-12:

TABLE 24
Pharmacokinetic of Oxycodone in Dogs Administered Oxycodone
Hydrochloride by Intranasal Insufflation at a Nominal Dose Level of
0.25 mg Oxycodone Free Base Equivalents/kg

| Oxycodone | | | | | | | |
|---|---|---|---|---|---|---|---|
| Pharmacokinetic | Dog No. | | | | | | |
| parameter | 741 | 743 | 745 | 747 | 749 | Mean | sd |
| Dose (mg/kg) | 0.199 | 0.243 | 0.197 | 0.227 | 0.217^{a} | 0.217 | 0.019 |
| Cₘₐₓ (ng/mL) | 17.9 | 76.0 | 26.9 | 122 | 165 | 81.6 | 62.6 |
| Cₘₐₓ/Dose | 22.5 | 78.2 | 34.1 | 134 | 190 | 91.8 | 70.3 |
| Tₘₐₓ (h) | 0.08 | 0.25 | 0.25 | 0.25 | 0.25 | 0.25^{b} | — |
| AUCₜ (ng · h/mL) | 24.8 | 91.2 | 31.6 | 97.8 | 112 | 71.5 | 40.3 |
| AUC (ng · h/mL) | 25.7 | 92.7 | 32.5 | 99.1 | 112 | 72.4 | 40.2 |
| AUC/Dose | 32.3 | 95.4 | 41.2 | 109 | 129 | 81.4 | 42.6 |
| t_{1/2} (h) | 1.28 | 1.45 | 1.17 | 1.40 | 1.71 | 1.382^{c} | — |
| T_{>50%Cmax} (h) | 1 | 0.25 | 0.25 | <0.25 | 0.25 | 0.25^{b} | — |
| F_{absolute}^{d}(%) | 32.5 | 77.6 | 42.5 | 124 | 129 | 81.1 | 44.7 |

^{a}Nominal dose level reported (value excluded from calculation of mean). Actual dose not quantifiable due to possible weighing error predose
^{b}Median value
^{c}Calculated as ln2/mean k
^{d}Calculated using individual AUC values following iv administration of oxymorphone HCl at a nominal dose of 0.25 mg/kg
Cₘₐₓ/Dose and AUC/Dose adjusted to a nominal dose of 0.25 mg/kg

TABLE 25
Pharmacokinetics of Oxycodone and Oxycodone [Succinyl-(S)-
Valine] Enol Ester in Dogs Administered Oxycodone [Succinyl-
(S)-Valine] Enol Ester TFA by Intranasal Insufflation at a Nominal
Dose Level of 0.25 mg Oxycodone Free Base Equivalents/kg

| Oxycodone | | | | | | | |
|---|---|---|---|---|---|---|---|
| Pharmacokinetic | Dog No. | | | | | | |
| parameter | 741 | 743 | 745 | 747 | 749 | Mean | sd |
| Dose (mg/kg) | 0.168 | 0.145 | 0.206 | 0.231 | 0.240 | 0.198 | 0.04 |
| Cₘₐₓ (ng/mL) | 3.05 | 4.65 | 8.43 | 3.48 | 5.95 | 5.11 | 2.17 |
| Cₘₐₓ/Dose | 4.54 | 8.01 | 10.2 | 3.77 | 6.20 | 6.54 | 2.61 |
| Tₘₐₓ (h) | 0.25 | 0.08 | 0.25 | 0.08 | 1.5 | 0.25^{a} | — |
| AUCₜ (ng · h/mL) | 4.86 | 8.01 | 18.1 | 4.94 | 18.6 | 10.9 | 6.9 |
| AUC (ng · h/mL) | 5.87 | 9.14 | 19.9 | 5.66 | 19.8 | 12.1 | 7.2 |
| AUC/Dose | 8.74 | 15.8 | 24.2 | 6.13 | 20.6 | 15.1 | 7.7 |
| t½ (h) | 2.31 | 1.92 | 1.96 | 1.50 | 1.89 | 1.88^{b} | — |
| T_{>50%Cmax} (h) | <0.25 | <0.25 | 1.5 | <0.25 | 3 | <0.25^{a} | — |
| Fᵣₑₗₐₜᵢᵥₑ^{c} (%) | 8.79 | 12.8 | 24.9 | 6.97 | 20.6 | 14.8 | 7.7 |
| Oxycodone [succinyl- | | | | | | | |
| (S)-valine] enol ester | | | | | | | |
| Pharmacokinetic | | | | | | | |
| parameter | 741 | 743 | 745 | 747 | 749 | Mean | sd |
| Cₘₐₓ (ng/mL) | 1.20 | 2.00 | 15.5 | 0.598 | 5.07 | 4.87 | 6.18 |
| Tₘₐₓ (h) | 0.25 | 0.50 | 0.25 | 1.00 | 0.25 | 0.25^{a} | — |
| AUCₜ (ng · h/mL) | 0.618 | 1.90 | 19.6 | 0.552 | 6.99 | 5.93 | 8.08 |
| AUC (ng · h/mL) | —^{d} | 2.65 | 20.4 | —^{d} | 7.92 | 10.3 | 9.1 |
| t½ (h) | —^{d} | 0.85 | 0.65 | —^{d} | 0.88 | 0.75^{b} | — |

^{a}Median value
^{b}Calculated as ln2/mean k
^{c}Calculated using individual AUC values following iv administration of oxymorphone HCl at a nominal dose of 0.25 mg/kg
^{d}Terminal phase could not be reliably determined
Cₘₐₓ/Dose and AUC/Dose adjusted to a nominal dose of 0.25 mg/kg

These results show a dramatically lower systemic availability of oxycodone following intranasal dosing with the succinyl valine ester prodrug compared with that seen after administration of the parent drug. Thus the oxycodone AUC value after the prodrug was only 18% of that seen after giving the parent drug while the Cmax value was just 7%. These lower oxycodone levels were not a reflection of good absorption and poor cleavage of the prodrug but due its inherent lack of intranasal absorbability. This was reflected by the very low levels of prodrug, as well as drug, seen in the plasma after intranasal dosing.

The minimal systemic exposure to oxycodone after giving the prodrug intranasally would be expected significantly minimize the risk of intranasal abuse of this oxycodone product.

### Example 24

### Comparative Assessment of the Systemic Availability of Hydrocodone in Dogs after Oral Administration of Either the Drug Itself or the Potential Prodrug Hydrocodone Succinyl Valine Ester

### Methodology

Test substances i.e. hydrocodone or hydrocodone succinyl valine enol ester were administered by oral gavage to a group of five male dogs in a two-way crossover design The characteristics of the test animals are set out in Table 26, below.

TABLE 26
Characteristics of Experimental Dogs Used in Study

| | |
|---|---|
| Species | Dog |
| Type | Beagle |
| Number and sex | 5 males |
| Approximate age | 6-12 months old |
| Approx. bodyweight | 8-12 kg, |
| Supplier | Harlan U.K. Ltd, Loughborough, U.K |

Blood samples were taken at various times after administration and submitted to analysis for the parent drug and pro-drug using a validated LC-MS-MS assay. Pharmacokinetic parameters derived from the plasma analytical data were determined using Win Nonlin.

### Results

Results are shown in Table 27-28

TABLE 27
Pharmacokinetics of Hydrocodone in Male Dogs Orally Administered
Hydrocodone at 1 mg Hydrocodone Free Base Equivalents/kg

| | Dog No. | | | | | | |
|---|---|---|---|---|---|---|---|
| Pharmacokinetic parameter | 1M | 2M | 3M | 4M | 5M | Mean | sd |
| Cₘₐₓ (ng/mL) | 119 | 76.8 | 57.8 | 53.3 | 71.1 | 75.6 | 26.1 |
| Tₘₐₓ (h) | 0.5 | 0.5 | 0.5 | 0.5 | 0.5 | 0.5* | |
| AUCₜ (ng · h/mL) | 154 | 107 | 89.2 | 69.7 | 94.1 | 103 | 31.5 |
| AUC (ng · h/mL) | 155 | 111 | 91.7 | 71.0 | 95.3 | 105 | 31.6 |
| t½ (h) | 1.31 | 1.58 | 1.66 | 1.66 | 1.46 | 1.54 | 0.15 |
| T_{>50%Cmax} (h) | 0.75 | 0.75 | 0.75 | 1.25 | 1.25 | 0.95 | 0.27 |

*Median value

TABLE 28
Pharmacokinetics of Hydrocodone in Dogs Orally Administered
Hydrodone [Succinyl-(S)-Valine] Enol Ester at 1.0 mg Hydrocodone
Free Base eEquivalents/kg

| Hydrocodone | | | | | | | |
|---|---|---|---|---|---|---|---|
| Pharmacokinetic | Dog No. | | | | | | |
| parameter | 1M | 2M | 3M | 4M | 5M | Mean | sd |
| Cₘₐₓ (ng/mL) | 73.4 | 62.5 | 37.8 | 35.5 | 40.5 | 49.9 | 17 |
| Tₘₐₓ (h) | 0.50 | 0.50 | 1.00 | 0.50 | 0.50 | 0.5* | |
| AUCₜ (ng · h/mL) | 123 | 86.5 | 53.6 | 45.6 | 54.8 | 72.7 | 32.2 |
| AUC (ng · h/mL) | 125 | 92.0 | 57.0 | 46.8 | 57.4 | 75.5 | 32.3 |
| t½ (h) | 1.21 | 0.92 | 1.02 | 0.69 | 0.83 | 0.93 | 0.19 |
| T_{>50%Cmax} (h) | 1.5 | 1.25 | 1.5 | 1.25 | 1.25 | 1.35 | 0.14 |
| Fᵣₑₗₐₜᵢᵥₑ (%) | 81 | 83 | 62 | 66 | 60 | 70 | |

*Median value

The results show a broadly similar systemic availability after giving either the parent drug or the prodrug. The mean relative value for the Cmax after giving the prodrug was 66% while the mean relative systemic exposure (AUC) was 70%.

### Example 25

### Stability and Comparative Bioavailability of Various Dicarboxylate Bridged Amino Acid Prodrugs of Meptazinol in Beagle Dogs and Cynomolgus Monkeys

### Methodology

Initially the inherent chemical and biological stability of various dicarboxylate bridged amino acid prodrugs of meptazinol, was investigated under the conditions prevailing in the GI tract. Premature hydrolysis of the prodrug in the gut lumen prior to absorption would reduce the opportunity for transient protection of the drug during its passage though the liver and the desired reduction in first pass metabolism.

These dicarboxylate amino acid enol esters, were incubated at 37° C. in simulated gastric and simulated intestinal juice (USP defined composition) for 1 and 2 hours respectively. The remaining concentrations of the prodrug (and also drug released) were then assayed by HPLC. Additionally their chemical stability was evaluated in pH 7.4 buffer for 2 h 37° C.

Subsequently, these various meptazinol dicarboxylate bridged amino acid prodrugs were administered by oral gavage to groups of two dogs and two monkeys at a standardized dose of 1 mg meptazinol base equivalents/kg body weight

Blood samples were taken at various times after administration and submitted to analysis for the parent drug and prodrug using a validated LC-MS-MS assay.

### Results

The results are presented in Table 29

TABLE 29
Stability and Plasma Pharmacokinetics of Meptazinol Dicarboxylate Linked Prodrugs

| | | | 2 h stability | 1 h stability in | 2 h stability in |
|---|---|---|---|---|---|
| | | | in pH7.4 | simulated | simulated |
| | Dog | Monkey | buffer | gastric fluid | intestinal |
| | Cₘₐₓ | Cₘₐₓ | (37° C.) | (37° C.) | fluid (37° C.) |
| | ng/mL | ng/mL | Conversion to meptazinol | | |
| Meptazinol [succinyl-(S)-valine] ester | 5.2 | — | 5% | — | — |
| Meptazinol-[3,3-dimethyl-glutaryl-(S)- | 1.2 (116) | 2.2 (43) | <1% | 2% | 1% |
| valine] ester | | | | | |
| Meptazinol [phythalyl-(S)-valine] ester | 20 (294) | 3.2 (51) | 52% | 2% | 29% |
| Meptazinol-[3,3-dimethylglutaryl-glycine] | 0.5 (29) | 1.1 (18) | 2% | 4% | 4% |
| ester | | | | | |
| Meptazinol [malearyl-(S)-valine] ester | 3.3 (6.0) | 1.4 (BLQ) | 53% | 8% | 16% |
| Meptazinol-[phthalyl-(S)-phenylalanine] | 9.1 (71) | 1.1 (BLQ) | 72% | 3% | 30% |
| ester | | | | | |
| Meptazinol-[fumaryl-(S)-valine] ester | 4.0 (BLQ) | 3.1 (BLQ) | 31% | 10% | ND |
| Meptazinol-[2,2-dimethylsuccinyl-(S)- | 0.9 (25) | 0.4 (100) | 2% | ND | 3% |
| valine] ester | | | | | |
| Meptazinol-[succinyl-(S)-proline] ester | 0.7 (36) | 2.0 (16) | 4% | 4% | 4% |
| Meptazinol [glutaryl-PABA] ester | 3.4 (6.7) | 1.5 (1.7) | 80% | 5% | 6% |
| Meptazinol | 9.4 (25) | 1.3 (BLQ) | 93% | 1% | 5% |
| [3,3-dimethyl-glutaryl-PABA]ester | | | | | |
| Meptazinol [fumaryl-PABA] ester | 1.5 (BLQ) | 1.4 (BLQ) | 47% | 2% | 6% |
| Meptazinol [malearyl-(S)-phenylanine] | 3.6 (1.7) | ND | ND | ND | ND |
| ester | | | | | |
| Meptazinol [malonyl-PABA] ester | 2.1 (11) | ND | ND | ND | ND |
| Meptazinol [malonyl-(S)-valine] ester | 2.4 (44) | ND | ND | ND | ND |
| Meptazinol [glutaryl-(S)-valine] ester | 17 (2.8) | ND | ND | ND | ND |

Concentration in brackets = plasma prodrug levels
BLQ = below limit of quantitation (0.5 ng/mL)
PABA = para-amino benzoic acid
ND = not determined

The results show that most of these dicarboxylate bridged amino acid prodrugs of meptazinol are generally quite stable under the conditions prevailing in the GI tract. The phthalyl valine and phenylalanine conjugates were however somewhat less stable showing some 30% degradation in 2 h in simulated intestinal fluid. Also some of these prodrugs including meptazinol [phythalyl-(S)-valine]ester and meptazinol [phythalyl-(S)-valine]ester showed chemical instability at pH 7.4 which could give beneficially rise to release of the active drug in blood.

Several of these prodrugs showed good systemic levels of the prodrug indicating efficient absorption e.g. meptazinol-[3,3-dimethyl-glutaryl-(S)-valine]ester and meptazinol [phythalyl-(S)-valine]ester. A number also showed improvement in systemic plasma levels over the expected Cmax (2-5 ng/mL) after giving the drug itself. The best performing conjugates, in terms of improved systemic levels of meptazinol, included meptazinol [phythalyl-(S)-valine]ester, meptazinol [glutaryl-(S)-valine] meptazinol [3,3-dimethyl-glutaryl-PABA]ester, and meptazinol-[phthalyl-(S)-phenylalanine]ester. These improvements while evident in the dog were, however, not seen in the monkey.

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. An oxycodone prodrug having the structure: 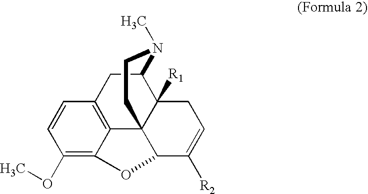
or a pharmaceutically acceptable salt thereof, wherein,
R₁ is independently selected from 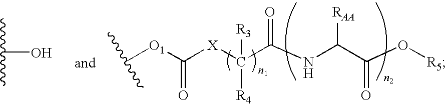
R₂ is selected from 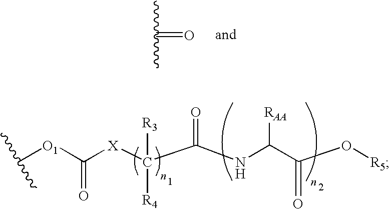
each occurrence of O₁ is independently an oxygen atom in the unbound form of oxycodone;
each occurrence of X is independently (—NH—), (—O—), or absent;
each occurrence of R₃ and R₄ is independently selected from hydrogen, alkoxy 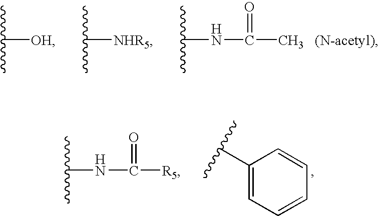
carboxyl, cycloalkyl, substituted cycloalkyl, alkyl, and substituted alkyl;
R₃ and R₄ on adjacent carbons can form a ring and R₃ and R₄ on the same carbon, taken together, can be a methylene group;
each occurrence of n₁ is independently an integer selected from 0 to 16 and each occurrence of n₂ is independently an integer selected from 1 to 9, and each occurrence of n₁ and n₂ can be the same or different;
the carbon chain defined by n₁ can include a cycloalkyl or aromatic ring;
in the case of a double bond in the carbon chain defined by n₁, R₃ is present and R₄ is absent on the carbons that form the double bond;
each occurrence of R₅ is independently selected from hydrogen, alkyl, substituted alkyl group and an opioid;
when R₅ is an opioid, the —O— is a hydroxylic oxygen present in the additional opioid R₅;
each occurrence of R_{AA} is independently selected from a proteinogenic or non-proteinogenic amino acid side chain;
the dashed line in Formula 2 is absent when R₂ is 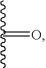
and a bond when R₂ is not 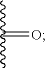
and
at least one of R₁ or R₂ is

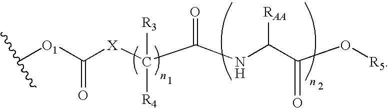

2. The oxycodone prodrug of claim 1 wherein n₁ is an integer selected from 0 to 4.

3. The oxycodone prodrug of claim 1 wherein R₂ is

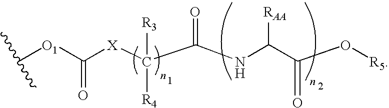

4. The oxycodone prodrug of claim 1 wherein X is absent and n₁ is 1, 2 or 3.

5. The oxycodone prodrug of claim 1 wherein R₁ is 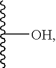
X is absent, n₁ is 0, 1, 2 or 3, n₂ is 1, 2 or 3, and R₃, R₄ and R₅ are each H.

6. The oxycodone prodrug of claim 5 wherein n₁ is 2.

7. The oxycodone prodrug of claim 1 wherein R₂ is 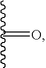
X is absent, n₁ is 0, 1, 2 or 3, n₂ is 1, 2 or 3 and R₃, R₄ and R₅ are each H.

8. The oxycodone prodrug of claim 7 wherein n₁ is 2.

9. The oxycodone prodrug of claim 1 wherein R₁ is 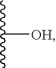
X is absent, n₁ is 0, 1, 2 or 3 n₂ is 1, 2, 3, 4 or 5, and R₃, R₄ and R₅ are each H.

10. The oxycodone prodrug of claim 9 wherein n₁ is 2.

11. The oxycodone prodrug of claim 1 wherein R₂ is 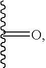
X is absent, n₁ is 0, 1, 2 or 3, n₂ is 1, 2, 3, 4 or 5, and R₃, R₄ and R₅ are each H.

12. The oxycodone prodrug of claim 11 wherein n₁ is 2.

13. The oxycodone prodrug of claim 1 wherein X is —O—, n₁ is 0, 1 or 2, n₂ is 1 or 2 and R₅ is H.

14. The oxycodone prodrug of claim 13 wherein n₁ is 2 and R₁ is

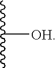

15. The oxycodone prodrug of claim 1 wherein X is —NH—, n₁ is 0, 1 or 2, n₂ is 1 or 2 and R₅ is H.

16. The oxycodone prodrug of claim 15 wherein n₁ is 2 and R₁ is

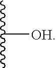

17. The oxycodone prodrug of claim 1 wherein n₁ is 1 or 2, n₂ is 1, 2 or 3, and R₅ is H.

18. The oxycodone prodrug of claim 1 wherein n₂ is 1, 2 or 3, and R₃, R₄ and R₅ are H.

19. The oxycodone prodrug of claim 18 wherein n₂ is 1.

20. The oxycodone prodrug of claim 18 wherein n₂ is 2.

21. The oxycodone prodrug of claim 18 wherein n₂ is 1 or 2 and each occurrence of R_{AA} is independently a proteinogenic amino acid side chain.

22. The oxycodone prodrug of claim 1 wherein X is —O—, n₁ is 1, 2, 3 or 4, n₂ is 1, or 3 and R₅ is H.

23. The oxycodone prodrug of claim 22 wherein at least one occurrence of R₃ is methyl.

24. The oxycodone prodrug of claim 1 wherein X is —NH—, n₁ is 0, 1 or 2, n₂ is 1 or 2 and R₅ is H.

25. The oxycodone prodrug of claim 1 wherein X is —NH—, n₁ is 1, 2, 3 or 4, n₂ is 1, 2 or 3 and R₅ is H.

26. The oxycodone prodrug of claim 25 wherein at least one occurrence of R₃ is methyl.

27. The oxycodone prodrug of claim 1 wherein X is absent, n₁ is 2, one occurrence of R₃ is —CH₃, and one occurrence of R₄ is —CH₃.

28. The oxycodone prodrug of claim 27 wherein R₅ is hydrogen.

29. The oxycodone prodrug of claim 27 wherein the one occurrence of R₃ and R₄ groups that are methyl occur on the same carbon atom.

30. The oxycodone prodrug of claim 1 wherein X is absent, n₁ is 2, and one occurrence of R₃ or R₄ is —CH₃.

31. The oxycodone prodrug of claim 30 wherein R₅ is hydrogen.

32. The oxycodone prodrug of claim 1 wherein X is absent, n₁ is 3, one occurrence of R₃ is —CH₃, and one occurrence of R₄ is —CH₃.

33. The oxycodone prodrug of claim 32 wherein R₅ is hydrogen.

34. The oxycodone prodrug of claim 32 wherein the one occurrence of R₃ and R₄ groups that are methyl occur on the same carbon.

35. The oxycodone prodrug of claim 1 wherein X is absent, n₁ is 2, and one occurrence of R₃ or R₄ is

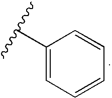

36. The oxycodone prodrug of claim 35 wherein R₅ is hydrogen.

37. The oxycodone prodrug of claim 1 wherein R_{AA} is the side chain of an amino acid selected from the group consisting of valine, leucine and isoleucine.

38. Oxycodone-[succinyl-valine] enol ester.

39. The composition of claim 38 comprising oxycodone-[succinyl-(S)-valine] enol ester.

40. The composition of claim 38 comprising oxycodone-[succinyl-(R)-valine] enol ester.

41. A pharmaceutical composition comprising oxycodone-[succinyl-valine] enol ester or a pharmaceutically acceptable salt thereof and a pharmaceutically acceptable carrier.

42. Oxycodone succinyl-leucine enol ester.

43. The composition of claim 42 comprising oxycodone-[succinyl-(S)-leucine] enol ester.

44. The composition of claim 42 comprising oxycodone-[succinyl-(R)-leucine] enol ester.

45. A pharmaceutical composition comprising oxycodone-[succinyl-leucine] enol ester or a pharmaceutically acceptable salt thereof and a pharmaceutically acceptable carrier.

46. Oxycodone-[glutaryl-valine] enol ester.

47. The composition of claim 46 comprising oxycodone-[glutaryl-(S)-valine] enol ester.

48. The composition of claim 46 comprising oxycodone-[glutaryl-(R)-valine] enol ester.

49. A pharmaceutical composition comprising oxycodone-[glutaryl-valine] enol ester or a pharmaceutically acceptable salt thereof and a pharmaceutically acceptable carrier.

50. Oxycodone-[glutaryl-leucine] enol ester.

51. The composition of claim 50 comprising oxycodone-[glutaryl-(S)-leucine] enol ester.

52. The composition of claim 50 comprising oxycodone-glutaryl-(R)-leucine enol ester.

53. A pharmaceutical composition comprising oxycodone-[glutaryl-leucine] enol ester or a pharmaceutically acceptable salt thereof and a pharmaceutically acceptable carrier.

54. A prodrug comprising oxycodone, a dicarboxylic acid linker, and a proteinogenic amino acid.

55. The prodrug of claim 54 wherein the dicarboxylic acid linker is succinic acid.

56. The prodrug of claim 54 wherein the dicarboxylic acid linker is glutaric acid.

57. The prodrug of claim 54 wherein the proteinogenic amino acid is valine.

58. The prodrug of claim 54 wherein the proteinogenic amino acid is leucine.

59. A method for reducing the incidence or severity of constipation associated with oral opiate administration which comprises orally administering to a patient in need thereof a prodrug comprising oxycodone, a dicarboxylic acid linker, and a proteinogenic amino acid.

60. The method of claim 59 wherein the prodrug is oxycodone-[succinyl-valine] enol ester.

61. The method of claim 59 wherein the prodrug is oxycodone-[succinyl-leucine] enol ester.

62. The method of claim 59 wherein the prodrug is oxycodone-[glutaryl-valine] enol ester.

63. The method of claim 59 wherein the prodrug is oxycodone-[glutaryl-leucine] enol ester.

64. A method for reducing the abuse of an opioid which comprises administering to a patient in need thereof a prodrug comprising oxycodone, a dicarboxylic acid linker, and a proteinogenic amino acid, wherein abuse of the prodrug by intranasal administration results in lower oxycodone absorption compared to intranasal administration of oxycodone itself.

65. The method of claim 64 wherein the prodrug is oxycodone-[succinyl-valine] enol ester.

66. The method of claim 64 wherein the prodrug is oxycodone-[succinyl-leucine] enol ester.

67. The method of claim 64 wherein the prodrug is oxycodone-[glutaryl-valine] enol ester.

68. The method of claim 64 wherein the prodrug is oxycodone-[glutaryl-leucine] enol ester.

69. A method of maintaining the plasma concentration of an opioid which comprises orally administering to a patient in need thereof a prodrug comprising oxycodone, a dicarboxylic acid linker, and a proteinogenic amino acid; wherein the plasma concentration of the oxycodone is sustained longer than the oxycodone plasma concentration following oral administration of oxycodone itself.

70. The method of claim 69 wherein the prodrug is oxycodone-[succinyl-valine] enol ester.

71. The method of claim 69 wherein the prodrug is oxycodone-[succinyl-(S)-leucine] enol ester.

72. The method of claim 69 wherein the prodrug is oxycodone-[glutaryl-(S)-valine] enol ester.

73. The method of claim 69 wherein the prodrug is oxycodone-[glutaryl-(S)-leucine] enol ester.

74. A method of improving the bioavailability of an opioid which comprises orally administering to a patient in need thereof a prodrug comprising oxycodone, a dicarboxylic acid linker, and a proteinogenic amino acid; wherein the bioavailability of the oxycodone is greater than the oxycodone bioavailability following oral administration of oxycodone itself.
